SEQUENCE LISTING (29 sequences)

SEQ ID NO: 1 - DNA, 19 bp, Artificial Sequence
  Synthetic oligonucleotide
tccatgagcttcctgagtc

SEQ ID NO: 2 - protein, 8 aa, Artificial Sequence
  Gallus gallus
SIINFEKL

SEQ ID NO: 3 - protein, 16 aa, Artificial Sequence
  Gallus gallus
TEWTSSNVMEERKIKV

SEQ ID NO: 4 - protein, 9 aa, Artificial Sequence
  HIV virus
AMQMLKETI

SEQ ID NO: 5 - protein, 19 aa, Artificial Sequence
  Homo Sapiens
GGSSLSYTNPAVAATSANL

SEQ ID NO: 6 - protein, 15 aa, Artificial Sequence
  Homo Sapiens
GEKETSATQRSSVPS

SEQ ID NO: 7 - protein, 9 aa, Artificial Sequence
  Homo Sapiens
SAPDNRPAL

SEQ ID NO: 8 - protein, 8 aa, Artificial Sequence
  HIV virus
AMQLKETI

SEQ ID NO: 9 - protein, 9 aa, Artificial Sequence
  HIV virus
YYPDSKDLI

SEQ ID NO: 10 - protein, 15 aa, Artificial Sequence
  HIV virus
IYKRWIILGLNKIVR

SEQ ID NO: 11 - protein, 15 aa, Artificial Sequence
  HIV virus
QWPLTEEKIKALVEI

SEQ ID NO: 12 - protein, 15 aa, Artificial Sequence
  HIV virus
EREVLEWRFDSRLAF

SEQ ID NO: 13 - protein, 13 aa, Artificial Sequence
  Artificial PADRE epitope
AKFVAAWTLKAAA

SEQ ID NO: 14 - DNA, 798 bp, Artificial Sequence
  Gallus gallus
gcggccgccatgggctccatcggtgcagcaagcatggaattttgttttgatgtattcaaggagctcatcaattcctgggtagaaagtcagacaaatggaattatcagaaatgtccttcagccaagctccgtggattctcaaactgcaatggttctggttaatgccattgtcttcaaaggactgtgggagaaaacatttaaggatgaagacacacaagcaatgcctttcagagtgactgagcaagaaagcaaacctgtgcagatgatgtaccagattggtttatttagagtggcatcaatggcttctgagaaaatgaagatcctggagcttccatttgccagtgggacaatgagcatgttggtgctgttgcctgatgaagtctcaggccttgagcagcttgagagtataatcaactttgaaaaactgactgaatggaccagttctaatgttatggaagagaggaagatcaaagtgtacttacctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgcaagcgtctctgaagaatttagggctgaccatccattcctcttctgtatcaagcacatcgcaaccaacgccgttctcttctttggcagatgtgtttccccttaaggatcc

SEQ ID NO: 15 - DNA, 449 bp, Artificial Sequence
  Mus
gctagcgccacaaccatgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacattgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccagtccaaaaatgaggcgcgcc

SEQ ID NO: 16 - DNA, 1818 bp, Artificial Sequence
  Homo Sapiens
gctagcgccaccatgtctagaacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggcccgccccgggctccaccgcccccccagcccacggtgtcacctcggccccggacaccaggcccgccccgggctccaccgcgcccgcagcccacggtgtcacctcggccccggacaccaggccggccccgggctccaccgccccccaagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtctagatagctcgag

SEQ ID NO: 17 - DNA, 20 bp, Artificial Sequence
  Artificial oligonucleotide
tccatgacgttcctgacgtt

SEQ ID NO: 18 - DNA, 18 bp, Artificial Sequence
  Artificial oligonucleotide
tctcccagcgtgcgccat

SEQ ID NO: 19 - DNA, 30 bp, Artificial Sequence
  Artificial oligonucleotide
accgatgacgtcgccggtgacggcaccacg

SEQ ID NO: 20 - DNA, 24 bp, Artificial Sequence
  Artificial oligonucleotide
tcgtcgttttgtcgttttgtcgtt

SEQ ID NO: 21 - DNA, 20 bp, Artificial Sequence
  Artificial oligonucleotide
tccatgacgttcctgatgct

SEQ ID NO: 22 - protein, 598 aa, Artificial Sequence
  Homo Sapiens
MSRTPGTQSPFFLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPAAHGVTSAPDTRPAPGSTAPQAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANLSR

SEQ ID NO: 23 - protein, 12 aa, Artificial Sequence
  Hepatitis B virus
PPAYRPPNAPIL

SEQ ID NO: 24 - DNA, 1878 bp, Artificial Sequence
  Homo Sapiens
gctagagccaccatggctagaacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggcccgccccgggctccaccgcccccccagcccacggtgtcacctcggccccggacaccaggcccgccccgggctccaccgcgcccgcagcccacggtgtcacctcggccccggacaccaggccggccccgggctccaccgccccccaagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtctagctggattcgcactcctccagcctatagaccaccaaatgcccctatcttatcaacacttccgtgactcgag

SEQ ID NO: 25 - DNA, 1857 bp, Artificial Sequence
  Homo Sapiens
gctagcgccaccatgtctagaacccctggcacccagtcccccttctttctcctgctgctgctcaccgtgctgaccgtcgtgaccggcagtgggcatgcgtcctcgacgcccggcggcgagaaggagaccagtgctacccagcgcagctctgtgccttccagcacggagaagaacgctgtgagtatgacttcctccgtgctgtcctcccatagccccggctcgggcagctccaccacccaggggcaggacgtgacactggctcccgcaaccgagcccgcctctggctctgccgccacctggggccaggacgtgacatccgtgcccgtgacccgccccgccctgggcagcaccaccccccctgctcatgacgtcacctcagccccggacaacaagccagccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggcccgccccgggctccaccgcccccccagcccacggtgtcacctcggccccggacaccaggcccgccccgggctccaccgcgcccgcagcccacggtgtcacctcggccccggacaccaggccggccccgggctccaccgccccccaagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcctccacactggtgcataacggaacctccgcgcgcgccaccaccaccccagcgagcaagagcacccccttctctatcccctcccatcatagcgacacacccaccacgctggccagccatagcaccaaaaccgacgcctctagcacccaccactccacggtgccccccctgacctccagcaaccattctacctccccccagctgtccacgggggtgagctttttcttcctgtccttccatatcagcaacctccagttcaattcctctctggaggaccccagcaccgactactaccaagagttgcagcgggacatctccgagatgttcctgcagatctacaaacagggcggcttcctgggattgagcaacatcaagttccgccccgggtccgtggtggtgcagctcaccctggccttcagggagggcaccatcaacgtgcatgacgtcgagacccagttcaatcagtataagaccgaggccgcctcccggtacaacctgacgatcagcgacgtgtctgtgtccgacgtgcccttccccttctccgcacagtccggcgccggcgtgcctggctggggcatcgccctgctcgtgttggtgtgcgtgctcgtggccctcgccatcgtgtacctgatcgccctggccgtctgtcagtgcaggagaaagaactatgggcagttggatatcttccccgccagggacacctaccaccccatgtccgagtaccccacctaccacacccacggccgctatgtccctccctcctcgaccgaccgctccccttacgagaaggtgagcgccggcaacggaggcagctccctgtcctacaccaaccctgccgtggccgccacaagcgccaacctgtctagagccaagttcgtggctgcctggaccctgaaggctgccgcttgactcgag

SEQ ID NO: 26 - DNA, 1857 bp, Artificial Sequence
  Homo Sapiens
gctagcgccaccatgtctagaacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggcccgccccgggctccaccgcccccccagcccacggtgtcacctcggccccggacaccaggcccgccccgggctccaccgcgcccgcagcccacggtgtcacctcggccccggacaccaggccggccccgggctccaccgccccccaagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtctagagccaagttcgtggctgcctggaccctgaaggctgccgcttgactcgag

SEQ ID NO: 27 - DNA, 3204 bp, Artificial Sequence
  HIV virus
atgggccccatcagtcccatcgagaccgtgccggtgaagctgaaacccgggatggacggccccaaggtcaagcagtggccactcaccgaggagaagatcaaggccctggtggagatctgcaccgagatggagaaagagggcaagatcagcaagatcgggccggagaacccatacaacacccccgtgtttgccatcaagaagaaggacagcaccaagtggcgcaagctggtggatttccgggagctgaataagcggacccaggatttctgggaggtccagctgggcatcccccatccggccggcctgaagaagaagaagagcgtgaccgtgctggacgtgggcgacgcttacttcagcgtccctctggacgaggactttagaaagtacaccgcctttaccatcccatctatcaacaacgagacccctggcatcagatatcagtacaacgtcctcccccagggctggaagggctctcccgccattttccagagctccatgaccaagatcctggagccgtttcggaagcagaaccccgatatcgtcatctaccagtacatggacgacctgtacgtgggctctgacctggaaatcgggcagcatcgcacgaagattgaggagctgaggcagcatctgctgagatggggcctgaccactccggacaagaagcatcagaaggagccgccattcctgaagatgggctacgagctccatcccgacaagtggaccgtgcagcctatcgtcctccccgagaaggacagctggaccgtgaacgacatccagaagctggtgggcaagctcaactgggctagccagatctatcccgggatcaaggtgcgccagctctgcaagctgctgcgcggcaccaaggccctgaccgaggtgattcccctcacggaggaagccgagctcgagctggctgagaaccgggagatcctgaaggagcccgtgcacggcgtgtactatgacccctccaaggacctgatcgccgaaatccagaagcagggccaggggcagtggacataccagatttaccaggagcctttcaagaacctcaagaccggcaagtacgcccgcatgaggggcgcccacaccaacgatgtcaagcagctgaccgaggccgtccagaagatcacgaccgagtccatcgtgatctgggggaagacacccaagttcaagctgcctatccagaaggagacctgggagacgtggtggaccgaatattggcaggccacctggattcccgagtgggagttcgtgaatacacctcctctggtgaagctgtggtaccagctcgagaaggagcccatcgtgggcgcggagacattctacgtggacggcgcggccaaccgcgaaacaaagctcgggaaggccgggtacgtcaccaaccggggccgccagaaggtcgtcaccctgaccgacaccaccaaccagaagacggagctgcaggccatctatctcgctctccaggactccggcctggaggtgaacatcgtgacggacagccagtacgcgctgggcattattcaggcccagccggaccagtccgagagcgaactggtgaaccagattatcgagcagctgatcaagaaagagaaggtctacctcgcctgggtcccggcccataagggcattggcggcaacgagcaggtcgacaagctggtgagtgcggggattagaaaggtgctgatggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgaatgggtgcccgagcttcggtactgtctggtggagagctggacagatgggagaaaattaggctgcgcccgggaggcaaaaagaaatacaagctcaagcatatcgtgtgggcctcgagggagcttgaacggtttgccgtgaacccaggcctgctggaaacatctgagggatgtcgccagatcctggggcaattgcagccatccctccagaccgggagtgaagagctgaggtccttgtataacacagtggctaccctctactgcgtacaccagaggatcgagattaaggataccaaggaggccttggacaaaattgaggaggagcaaaacaagagcaagaagaaggcccagcaggcagctgctgacactgggcatagcaaccaggtatcacagaactatcctattgtccaaaacattcagggccagatggttcatcaggccatcagcccccggacgctcaatgcctgggtgaaggttgtcgaagagaaggccttttctcctgaggttatccccatgttctccgctttgagtgagggggccactcctcaggacctcaatacaatgcttaataccgtgggcggccatcaggccgccatgcaaatgttgaaggagactatcaacgaggaggcagccgagtgggacagagtgcatcccgtccacgctggcccaatcgcgcccggacagatgcgggagcctcgcggctctgacattgccggcaccacctctacactgcaagagcaaatcggatggatgaccaacaatcctcccatcccagttggagaaatctataaacggtggatcatcctgggcctgaacaagatcgtgcgcatgtactctccgacatccatccttgacattagacagggacccaaagagccttttagggattacgtcgaccggttttataagaccctgcgagcagagcaggcctctcaggaggtcaaaaactggatgacggagacactcctggtacagaacgctaaccccgactgcaaaacaatcttgaaggcactaggcccggctgccaccctggaagagatgatgaccgcctgtcagggagtaggcggacccggacacaaagccagagtgttg

SEQ ID NO: 28 - DNA, 435 bp, Artificial Sequence
  Homo Sapiens
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtga

SEQ ID NO: 29 - protein, 144 aa, Artificial Sequence
  Homo Sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

(19) United States
(12) Patent Application Publication
(10) Pub. No.: US 2008/0145375 A1
(43) Pub. Date: Jun. 19, 2008

(54) Vaccination

(75) Inventors: Gary Peter Bembridge, Hertfordshire (GB); Jennifer L Craigen, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited, Greenford, Middlesex (GB)

(21) Appl. No.: 10/571,812

(22) PCT Filed: Sep. 13, 2004
(86) PCT No.: PCT/EP04/10322

§ 371 Date: Dec. 11, 2006

(30) Foreign Application Priority Data
Sep. 15, 2003 (GB) 0321615.7

(51) Int. Cl. A61K 39/39; A61P 35/00

(52) U.S. Cl. 424/184.1; 424/278.1

(57) ABSTRACT

The present invention relates to improved nucleic acid vaccines, adjuvant systems, and processes for the preparation of such vaccines and adjuvant systems. In particular, the nucleic acid vaccines and adjuvant systems of the present invention comprise a combination of a nucleotide sequence encoding GM-CSF, or derivatives thereof, and toll-like receptor (TLR) agonists, or derivatives thereof.

### FIELD OF THE INVENTION

The present invention relates to improved nucleic acid vaccines, adjuvant systems, and processes for the preparation of such vaccines and adjuvant systems. In particular, the nucleic acid vaccines and adjuvant systems of the present invention comprise a combination of a nucleotide sequence encoding GM-CSF, or derivatives thereof, and toll-like receptor (TLR) agonists, or derivatives thereof.

### BACKGROUND OF THE INVENTION

Traditional vaccination techniques which involve the introduction into an animal system of an antigen which can induce an immune response in the animal, and thereby protect the animal against infection, have been known for many years. Following the observation in the early 1990's that plasmid DNA could directly transfect animal cells in vivo, significant research efforts have been undertaken to develop vaccination techniques based upon the use of DNA plasmids to induce immune responses, by direct introduction into animals of DNA which encodes for antigenic peptides. Such techniques, which are referred to as “DNA immunisation” or “DNA vaccination” have now been used to elicit protective antibody (humoral) and cell-mediated (cellular) immune responses in a wide variety of pre-clinical models for viral, bacterial and parasitic diseases. Research is also underway in relation to the use of DNA vaccination techniques in treatment and protection against cancer, allergies and autoimmune diseases.

DNA vaccines usually consist of a bacterial plasmid vector into which is inserted a strong promoter, the gene of interest which encodes for an antigenic peptide and a polyadenylation/transcriptional termination sequence. The immunogen which the gene of interest encodes may be a full protein or simply an antigenic peptide sequence relating to the pathogen, tumour or other agent which is intended to be protected against. The plasmid can be grown in bacteria, such as for example E. coli and then isolated and prepared in an appropriate medium, depending upon the intended route of administration, before being administered to the host.

Helpful background information in relation to DNA vaccination is provided in “Donnelly, J et al Annual Rev. Immunol. (1997) 15:617-648; Ertl P. and Thomsen L., Technical issues in construction of nucleic acid vaccines Methods. 2003 November; 31(3):199-206; the disclosures of which are included herein in their entirety by way of reference.

There are a number of advantages of DNA vaccination relative to traditional vaccination techniques. First, it is predicted that because the proteins which are encoded by the DNA sequence are synthesised in the host, the structure or conformation of the protein will be similar to the native protein associated with the disease state. It is also likely that DNA vaccination will offer protection against different strains of a virus, by generating cytotoxic T lymphocyte responses that recognise epitopes from conserved proteins. Furthermore, because the plasmids are introduced directly to host cells where antigenic protein can be produced, a long-lasting immune response will be elicited. The technology also offers the possibility of combining diverse immunogens into a single preparation to facilitate simultaneous immunisation in relation to a number of disease states.

Despite the numerous advantages associated with DNA vaccination relative to traditional vaccination therapies, there is nonetheless a desire to develop adjuvant compounds which will serve to increase the immune response induced by the protein which is encoded by the plasmid DNA administered to an animal.

DNA vaccination is sometimes associated a deviation of immune response from a Th1 to a Th2 response, especially when the DNA is administered directly to the epidermis (Fuller and Haynes, Hum. Retrovir. (1994) 10:1433-41). It is recognised that the immune profile desired from a nucleic acid vaccine depends on the disease being targeted. The preferential stimulation of a Th1 response is likely to provide efficacy of vaccines for many viral diseases and cancers, and a dominant Th2 type of response may be effective in limiting allergy and inflammation associated with some autoimmune diseases. Accordingly, ways to quantitatively raise the immune response or to shift the type of response to that which would be most efficacious for the disease indication, may be useful.

Dendritic cells are present in an immature form in tissues. In response to infection of the tissue or other tissue damage, dendritic cells migrate towards the damaged tissue, where they take up, process and present peptides from the damaged tissue and migrate to the lymph nodes. The peptides are presented by the dendritic cells in the context of surface major histocompatibility complex (MHC) molecules, together with costimulatory molecules. Dendritic cells presenting peptide in the MHC together with costimulatory molecules are termed “mature” dendritic cells. Mature dendritic cells are able to interact with T cells, and activate T cells which recognise presented peptide to mount an immune response to eliminate the cause of the tissue damage (for example, invading bacteria).

Granulocyte-macrophage colony stimulating factor (GM-CSF) is a cytokine capable of inducing differentiation, proliferation and activation of a range of cells with immunological function. GM-CSF induces proliferation of dendritic cells from bone marrow precursors to reach an immature dendritic cell state, ie the cells express low levels of co-stimulatory markers and high levels of receptors for antigen uptake.

Toll-like receptors (TLRs) are type I transmembrane receptors, evolutionarily conserved between insects and humans. Ten TLRs have so far been established (TLRs 1-10) (Sabroe et al, JI 2003 p 1630-5). Members of the TLR family have similar extracellular and intracellular domains; their extracellular domains have been shown to have leucine-rich repeating sequences, and their intracellular domains are similar to the intracellular region of the interleukin-1 receptor (IL-1R). TLR cells are expressed differentially among immune cells and other cells (including vascular epithelial cells, adipocytes, cardiac myocytes and intestinal epithelial cells). The intracellular domain of the TLRs can interact with the adaptor protein Myd88, which also posses the IL-1R domain in its cytoplasmic region, leading to NF-KB activation of cytokines; this Myd88 pathway is one way by which cytokine release is effected by TLR activation. The main expression of TLRs is in cell types such as antigen presenting cells (eg dendritic cells, macrophages etc).

Activation of dendritic cells by stimulation through the TLRs leads to maturation of dendritic cells, and production of inflammatory cytokines such as IL-12. Research carried out so far has found that TLRs recognise different types of agonists, although some agonists are common to several TLRs. TLR agonists are predominantly derived from bacteria or viruses, and include molecules such as flagellin or bacterial lipopolysaccharide (LPS).

The imidazoquinoline compounds imiquimod and resiquimod are small anti-viral compounds. Imiquimod has been used for the local treatment of genital warts caused by human papilloma virus; resiquimod has also been tested for use in treatment of genital warts. Imiquimod and resiquimod are believed to act through the TLR-7 and/or TLR-8 signalling pathways and activation of the Myd88 activation pathway.

The present inventors have identified certain adjuvant combinations which are effective in promoting an improved immune response, in particular an improved cellular immune response when used as adjuvants in DNA vaccination.

### STATEMENT OF INVENTION

According to one embodiment of the present invention there is provided an adjuvant composition comprising:

(i) a TLR agonist, or nucleotide sequence encoding a TLR agonist; and
(ii) a nucleotide sequence encoding GM-CSF
in which components (i) and (ii) act in functional co-operation to enhance the immune responses in a mammal to an antigen.

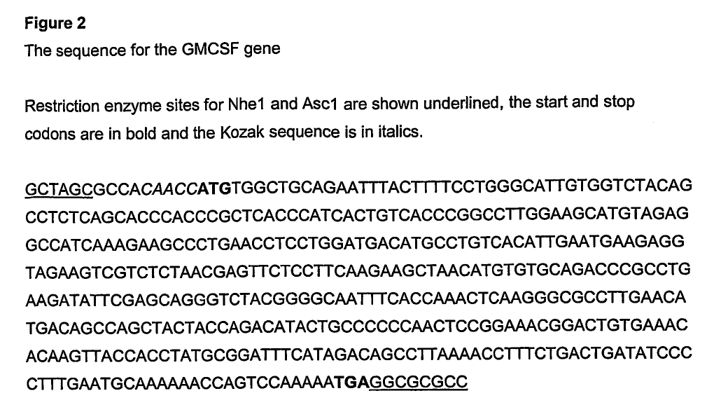

By GM-CSF is meant the entire molecule of GM-CSF or any fragment thereof capable of inducing proliferation of bone marrow precursor cells to reach an immature dendritic cell state. The polynucleotide gene sequence of mouse GM-CSF is shown in FIG. 2. The DNA sequence for human GM-CSF was obtained from the Genbank database (accession number M11220—Ref. Lee, F. et al PNAS 82(13) 4360-4364 (1985)).

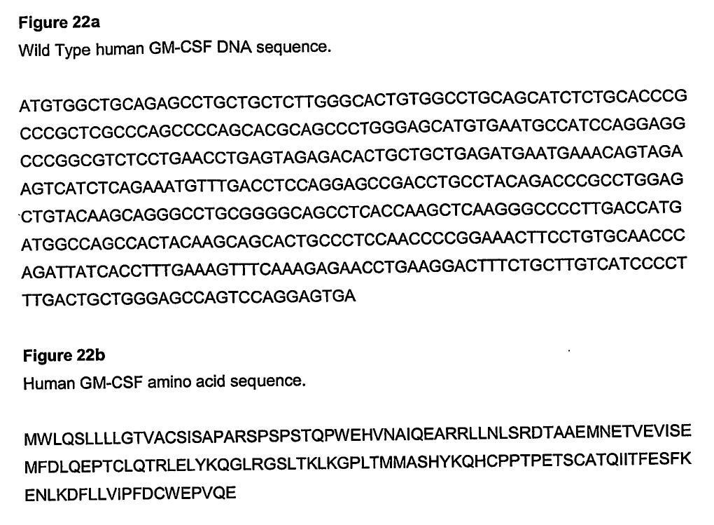

In one embodiment, where the adjuvants are for use in human vaccines, the GM-CSF sequence is the human sequence (see FIG. 22).

The nucleotide sequences of the present invention, for example the nucleotide sequence encoding GM-CSF, may be provided within the context of a plasmid comprising regulatory control sequences. For example, the nucleotide sequence may be within the context of vaccine vector p7313 (details included in WO 02/08435) under the regulatory control of human cytomegalovirus (CMV) immediate early (1E) promoter.

By “TLR agonist” it is meant a component which is capable of causing a signalling response through a TLR signalling pathway, either as a direct ligand or indirectly through generation of endogenous or exogenous ligand (Sabroe et al, JI 2003 p 1630-5).

In one embodiment of the present invention, component (i) is a TLR agonist capable of causing a signalling response through TLR-1 (Sabroe et al, JI 2003 p 1630-5). In one embodiment, the TLR agonist capable of causing a signalling response through TLR-1 is selected from: Tri-acylated lipopeptides (LPs); phenol-soluble modulin; Mycobacterium tuberculosis LP; S-(2,3-bis(palmitoyloxy)-(2-RS)-propyl)-N-palmitoyl-(R)-Cys-(S)-Ser-(S)-Lys(4)-OH, trihydrochloride (Pam₃Cys) LP which mimics the acetylated amino terminus of a bacterial lipoprotein and OspA LP from Borrelia burgdorfei.

In an alternative embodiment, component (i) is a TLR agonist capable of causing a signalling response through TLR-2 (Sabroe et al, JI 2003 p 1630-5). In one embodiment, the TLR agonist capable of causing a signalling response through TLR-2 is one or more of a bacterial lipopeptide from M tuberculosis, B burgdorferi, T pallidum; peptidoglycans from species including Staphylococcus aureus; lipoteichoic acids, mannuronic acids, Neisseria porins, bacterial fimbriae, Yersina virulence factors, CMV virions, measles haemagglutinin, and zymosan from yeast.

In an alternative embodiment, component (i) is a TLR agonist capable of causing a signalling response through TLR-3 (Sabroe et al, JI 2003 p 1630-5). In one embodiment, the TLR agonist capable of causing a signalling response through TLR-3 is double stranded RNA, or polyinosinic-polycytidylic acid (Poly IC), a molecular nucleic acid pattern associated with viral infection.

In an alternative embodiment, component (i) is a TLR agonist capable of causing a signalling response through TLR-4 (Sabroe et al, JI 2003 p 1630-5). In one embodiment, the TLR agonist capable of causing a signalling response through TLR-4 is one or more of a lipopolysaccharide (LPS) from gram-negative bacteria, or fragments thereof; heat shock protein (HSP) 10, 60, 65, 70, 75 or 90; surfactant Protein A, hyaluronan oligosaccharides, heparan sulphate fragments, fibronectin fragments, fibrinogen peptides and b-defensin-2. In one embodiment the TLR agonist is HSP 60, 70 or 90. In an alternative embodiment, the TLR agonist capable of causing a signalling response through TLR-4 is a non-toxic derivative of LPS. Monophosphoryl lipid A (MPL), is one such non-toxic derivative, produced by removal of the core carbohydrate group and the phosphate from the reducing-end glucosamine. MPL has been described by Ribi et al (1986, Immunology and Immunopharmacology of bacterial endotoxins, Plenum Publ. Corp., NY, p 407-419). MPL, which may be used as a TLR agonist in the present invention, has the following structure:

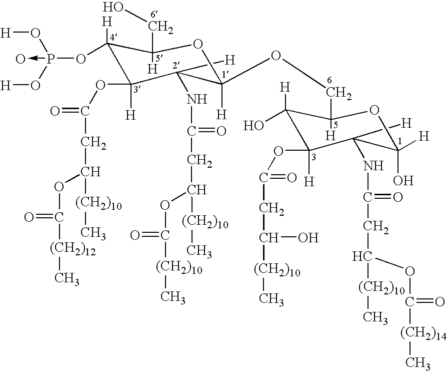

A further detoxified version of MPL results from the removal of the acyl chain from the 3-position of the disaccharide backbone, and is called 3-O-Deacylated monophosphoryl lipid A (3D-MPL). 3D-MPL is a TLR agonist which may be used in the present invention. It can be purified and prepared by the methods taught in GB 2122204B, which reference also discloses the preparation of diphosphoryl lipid A, and 3-O-deacylated variants thereof. A form of 3D-MPL is in the form of an emulsion having a small particle size less than 0.2 μm in diameter, and its method of manufacture is disclosed in WO 94/21292. Aqueous formulations comprising monophosphoryl lipid A and a surfactant have been described in WO9843670A2. Other purified and synthetic non-toxic derivatives of LPS have been described (U.S. Pat. No. 6,005,099 and EP 0 729 473 B1; Hilgers et al., 1986, Int. Arch. Allergy. Immunol., 79(4):392-6; Hilgers et al., 1987, Immunology, 60(1):141-6; and EP 0 549 074 B1).

The non-toxic derivatives of LPS, or bacterial lipopolysaccharides, which may be used as TLR agonists in the present invention may be purified and processed from bacterial sources, or alternatively they may be synthetic. For example, purified monophosphoryl lipid A is described in Ribi et al 1986 (supra), and 3-O-Deacylated monophosphoryl or diphosphoryl lipid A derived from Salmonella sp. is described in GB 2220211 and U.S. Pat. No. 4,912,094. Other purified and synthetic lipopolysaccharides have been described (U.S. Pat. No. 6,005,099 and EP 0 729 473 B1; Hilgers et al., 1986, Int. Arch. Allergy. Immunol., 79(4):392-6; Hilgers et al., 1987, Immunology, 60(1):141-6; and EP 0 549 074 B1). Bacterial lipopolysaccharide adjuvants may be 3D-MPL and the β(1-6) glucosamine disaccharides described in U.S. Pat. No. 6,005,099 and EP 0 729 473 B1.

Accordingly, other LPS derivatives that may be used as TLR agonists in the present invention are those immunostimulants that are similar in structure to that of LPS or MPL or 3D-MPL. In another aspect of the present invention the LPS derivatives may be an acylated monosaccharide, which is a sub-portion to the above structure of MPL.

A disaccharide agonist may be a purified or synthetic lipid A of the following formula:

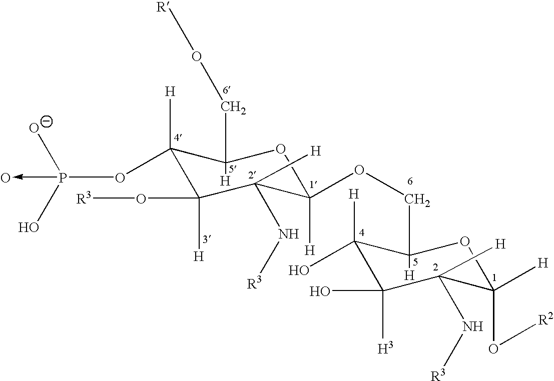

wherein R2 may be H or PO3H2; R3 may be an acyl chain or β-hydroxymyristoyl or a 3-acyloxyacyl residue having the formula:

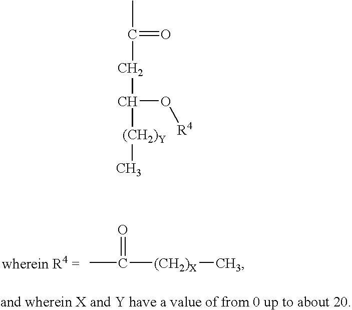

A yet further non-toxic derivative of LPS, which shares little structural homology with LPS and is purely synthetic is that described in WO 00/00462, the contents of which are fully incorporated herein by reference.

In an alternative embodiment, component (i) is a TLR agonist capable of causing a signalling response through TLR-5 (Sabroe et al, JI 2003 p 1630-5). In one embodiment, the TLR agonist capable of causing a signalling response through TLR-5 is bacterial flagellin.

In an alternative embodiment, component (i) is a TLR agonist capable of causing a signalling response through TLR-6 (Sabroe et al, JI 2003 p 1630-5). In one embodiment, the TLR agonist capable of causing a signalling response through TLR-6 is mycobacterial lipoprotein, di-acylated LP, and phenol-soluble modulin. Further TLR6 agonists are described in WO2003043572.

In an alternative embodiment, component (i) is a TLR agonist capable of causing a signalling response through TLR-7 (Sabroe et al, JI 2003 p 1630-5). In one embodiment, the TLR agonist capable of causing a signalling response through TLR-7 is loxoribine, a guanosine analogue at positions N7 and C8, or an imidazoquinoline compound, or derivative thereof. In one embodiment, the TLR agonist is imiquimod. Further TLR7 agonists are described in WO02085905.

In an alternative embodiment, component (i) is a TLR agonist capable of causing a signalling response through TLR-8 (Sabroe et al, JI 2003 p 1630-5). In one embodiment, the TLR agonist capable of causing a signalling response through TLR-8 is an imidazoquinoline molecule with anti-viral activity, for example resiquimod (R848); resiquimod is also capable of recognition by TLR-7. Other TLR-8 agonists which may be used include those described in WO2004071459.

In an alternative embodiment, the TLR agonist is imiquimod. In another embodiment the TLR agonist is resiquimod.

In an alternative embodiment, component (i) is a TLR agonist capable of causing a signalling response through TLR-9 (Sabroe et al, JI 2003 p 1630-5). In one embodiment, the TLR agonist capable of causing a signalling response through TLR-9 is HSP90. Alternatively, the TLR agonist capable of causing a signalling response through TLR-9 is DNA containing unmethylated CpG nucleotides, in particular sequence contexts known as CpG motifs.

CpG-containing oligonucleotides induce a predominantly Th1 response. Such oligonucleotides are well known and are described, for example, in WO 96/02555, WO 99/33488 and U.S. Pat. Nos. 6,008,200 and 5,856,462.

In one embodiment, CpG nucleotides are CpG oligonucleotides.

In one embodiment, the CpG nucleotide is an oligonucleotide composition having an immunostimulatory oligonucleotide region containing at least one CG unmethylated di-nucleotide motif. The immunostimulatory sequence is often: Purine, Purine, C, G, pyrimidine, pyrimidine; wherein the dinucleotide CG motif is not methylated.

In one embodiment, CpG nucleotides contain two or more dinucleotide CpG motifs separated by at least three, or at least six or more nucleotides. The CpG nucleotides of the present invention are typically deoxynucleotides.

In one embodiment the internucleotide bond in the oligonucleotide is phosphorodithioate, In a further embodiment the internucleotide bond in the oligonucleotide is a phosphorothioate bond, although phosphodiester and other internucleotide bonds are within the scope of the invention including oligonucleotides with mixed internucleotide linkages. Methods for producing phosphorothioate oligonucleotides or phosphorodithioate are described in U.S. Pat. No. 5,666,153, U.S. Pat. No. 5,278,302 and WO95/26204.

Examples of CpG nucleotides have the following sequences. The sequences may contain phosphorothioate modified internucleotide linkages.

OLIGO 1 (SEQ ID NO:17):
TCC ATG ACG TTC CTG ACG TT (CpG 1826)
OLIGO 2 (SEQ ID NO:18):
TCT CCC AGC GTG CGC CAT (CpG 1758)
OLIGO 3 (SEQ ID NO:19):
ACC GAT GAC GTC GCC GGT GAC GGC ACC ACG
OLIGO 4 (SEQ ID NO:20):
TCG TCG TTT TGT CGT TTT GTC GTT (OpG 2006)
OLIGO 5 (SEQ ID NO:21):
TCC ATG ACG TTC CTG ATG CT (CpG 1668)

Alternative CpG oligonucleotides may comprise the sequences above in that they have inconsequential deletions or additions thereto.

The CpG nucleotides utilised in the present invention may be synthesised by any method known in the art (e.g. EP 468520). Conveniently, such CpG nucleotides may be synthesised utilising an automated synthesiser.

The CpG nucleotides utilised in the present invention are typically deoxynucleotides. In one embodiment the internucleotide bond in the oligonucleotide is a phosphorodithioate.

In a further embodiment the internucleotide bond in the oligonucleotide is a phosphorothioate bond, although phosphodiesters are within the scope of the present invention. Oligonucleotide comprising different internucleotide linkages are contemplated, e.g. mixed phosphorothioate phosphodiesters. Other internucleotide bonds which stabilise the oligonucleotide may be used.

In an alternative embodiment, component (i) is a TLR agonist capable of causing a signalling response through TLR-10. Alternatively, the TLR agonist is capable of causing a signalling response through any combination of two or more of the above TLRs.

Particular TLR agonists which may be used in the present invention include agonists of TLRs 2, 4, 7 or 8.

In a further alternative embodiment, combinations of more than one TLR agonist may be used. In one embodiment of the present invention, an agonist of TLR-4 and an agonist of TLR-7 are used.

In one embodiment of the present invention, component (i) is not capable of causing a signalling response through TLR-9.

The present invention is not limited to the TLR-agonists listed herein; other natural ligands or synthetic TLR agonists may also be used in the present invention.

In an embodiment of the present invention, the TLR agonist is capable of causing a signalling response through TLR-7. In one embodiment of the present invention, the TLR agonist is an imidazoquinoline compound, or derivative thereof. In a further embodiment, the imidazoquinoline or derivative thereof is a compound defined by any one of formulae I-VI, as defined herein. In a further embodiment, the imidazoquinoline or derivative thereof is a compound defined by formula VI. In one embodiment, the imidazoquinoline or derivative thereof is a compound of formula VI selected from the group consisting of
1-(2-methylpropyl)-1H-imidazo[4,5-c]quinolin-4-amine;
1-(2-hydroxy-2-methylpropyl)-2-methyl-1H-imidazo[4,5-c]quinolin-4-amine;
1-(2,hydroxy-2-methylpropyl)-1H-imidazo[4,5-c]quinolin-4-amine;
1-(2-hydroxy-2-methylpropyl)-2-ethoxymethyl-1-H-imidazo[4,5-c]quinolin-4-amine

In a further embodiment the imidazoquinoline or derivative thereof is imiquimod or resiquimod. The imidazoquinoline or derivative thereof may be imiquimod. In one embodiment of the present invention, when the imidazoquinoline or derivative thereof is imiquimod, the imiquimod is provided in a cream formulation for topical administration. An example of a cream formulation of imiquimod which may be used is Aldara™ cream 5% (3M). In an alternative embodiment of the present invention, when the imidazoquinoline or derivative thereof is resiquimod, the resiquimod is provided in a formulation for oral administration, or intradermal, administration. In one embodiment of the present invention, components (ii) and (iii) are polynucleotide sequences which are administered concomitantly, and component (i) is an imidazoquinoline, for example imiquimod, which is administered topically, for example in a cream formulation, between 12 and 36 hours after administration of components (ii) and (iii), for example at or about 24 hours after administration of components (ii) and (iii).

In one embodiment of the present invention, the nucleotide sequences encoding components (i), (ii) or (iii) of the present invention are DNA. In a further embodiment, the nucleotide sequence or polynucleotide molecule is encoded within plasmid DNA

In one embodiment of the adjuvant composition of the present invention, the nucleotide sequences encoding component (i) and component (ii) are co-encoded within one plasmid

In one embodiment, adjuvant component (i) is a nucleotide sequence encoding one or more of the following, or encoding a component of the following capable of acting as a TLR agonist: β-defensin; HSP60; HSP70; HSP90 or other lower molecular weight HSP capable of acting as a TLR agonist; fibronectin; and flagellin protein

In an alternative embodiment, the TLR agonist of adjuvant component (i) is one or more of the following, or a component of the following, capable of acting as a TLR agonist:

a TLR-1 agonist such as: Tri-acylated lipopeptides (LPs); phenol-soluble modulin; Mycobacterium tuberculosis LP; S-(2,3-bis(palmitoyloxy)-(2-RS)-propyl)-N-palmitoyl-(R)-Cys-(S)-Ser-(S)-Lys(4)-OH, trihydrochloride (Pam₃Cys) LP which mimics the acetylated amino terminus of a bacterial lipoprotein and OspA LP from Borrelia burgdorfei;
a TLR-2 agonist such as: a bacterial lipopeptide from M tuberculosis, B burgdorferi, T pallidum; peptdoglycans from species including Staphylococcus aureus; lipoteichoic acids, mannuronic acids, Neisseria porins, bacterial fimbriae, Yersina virulence factors, CMV virions, measles haemagglutinin, and zymosan from yeast;
a TLR-3 agonist such as: double stranded RNA, or polyinosinic-polycytidylic acid (Poly IC), a molecular nucleic acid pattern associated with viral infection;
a TLR-4 agonist such as: a lipopolysaccharide (LPS) from gram-negative bacteria, or fragments thereof; heat shock protein (HSP) 10, 60, 65, 70, 75 or 90; surfactant Protein A, hyaluronan oligosaccharides, heparan sulphate fragments, fibronectin fragments, fibrinogen peptides and b-defensin-2, or a non-toxic derivative of LPS such as monophosphoryl lipid A (MPL);
a TLR-5 agonist such as: bacterial flagellin;
a TLR-6 agonist such as: mycobacterial lipoprotein, di-acylated LP, and phenol-soluble modulin;
a TLR-7 agonist such as: loxoribine, a guanosine analogue at positions N7 and C8, or an imidazoquinoline compound, or derivative thereof such as imiquimod or resiquimod;
a TLR-8 agonist such as: an imidazoquinoline molecule with anti-viral activity, such as resiquimod;
a TLR-9 agonist such as: HSP90 or DNA containing unmethylated CpG nucleotides, in particular sequence contexts known as CpG motifs.
for concomitant or sequential administration with component (ii). In one embodiment, component (i) is one of the preceding TLR agonists.

The present invention further provides an immunogenic composition or compositions comprising adjuvant components (i) and (ii) as described herein, and

(iii) an immunogen component comprising a nucleotide sequence encoding an antigenic peptide or protein

In one embodiment of the present invention, component (i) is encoded by a nucleotide sequence, and the nucleotide sequences encoding components (i), (ii) and (iii) are comprised or consist within one, or the same, polynucleotide molecule

In a further embodiment of the present invention, component (i) is encoded by a nucleotide sequence, and the nucleotide sequences encoding components (i), (ii) and (iii) are comprised or consist within separate polynucleotide molecules, for concomitant or sequential administration

Alternatively, nucleotide sequences encoding any two of the components (i), (ii) and (iii) may comprise or consist within one, or the same, polynucleotide molecule, and the remaining nucleotide sequence may be encoded within a further polynucleotide molecule, for concomitant or sequential administration. The nucleotide sequences encoding components (ii) and (iii) may be comprised or may consist within one, or the same, polynucleotide molecule, and the nucleotide sequence encoding component (i) may be encoded within a further polynucleotide molecule, for concomitant or sequential administration

In an embodiment of the invention where components (i), (ii) and/or (iii) are comprised or consist within separate polynucleotide molecules, the polynucleotide molecules may each be present within separate plasmids for concomitant or sequential delivery. In one embodiment, concomitant delivery may be used.

In one embodiment of the present invention, the nucleotide sequence encoding component (i) and the nucleotide sequence encoding component (ii), are comprised or consist within one, or the same, polynucleotide molecule

In an alternative embodiment, the nucleotide sequence encoding component (i) and the nucleotide sequence encoding component (ii) are encoded by nucleotide sequences which are comprised or consist within different nucleotide molecules, for concomitant or sequential administration.

By concomitant administration is meant substantially simultaneous administration; that is, components are administered at the same time, or if not, at least within a few minutes of each other. Alternatively, components are administered within one, two, three, four, five or 10 minutes of each other. In one treatment protocol, adjuvant components (I) and (ii) are administered substantially simultaneously to administration of the nucleotide sequence encoding immunogen (iii). Obviously, this protocol can be varied as necessary

In one embodiment of the present invention, component (i) is an imidazoquinoline or derivative thereof, and is provided in a separate composition from components (ii) and (iii) for concomitant or sequential administration. In one embodiment, the imidazoquinoline compound, or derivative thereof is administered sequentially, that is after the administration of components (ii) and (iii), in a separate composition. In a further embodiment, the imidazoquinoline compound, or derivative thereof, is given 2, 4, 6, 8, 12 or 24 hours after administration of components (ii) and (iii). In one embodiment, the imidazoquinoline compound or derivative thereof is given at or about 24 hours after administration of components (ii) and (iii). In a further embodiment, where the imidazoquinoline compound, or derivative thereof is for topical administration, in a cream formulation, the cream is applied 24 hours after administration of components (ii) and (iii). In an alternative embodiment of the present invention, where the imidazoquinoline compound, or derivative thereof is provided in a soluble formulation for administration, for example but not limited to sub-cutaneous administration, the imidazoquinoline compound, or derivative thereof may be administered between 6 and 24 hours after administration of components (ii) and (iii), or may be administered the next working day after administration of components (ii) and (iii). Components (ii) and (iii) may be packaged onto a gold bead and administered into the skin of a patient using particle mediated drug delivery, for example using a “gene gun” as described in, for example, EP0500799.

In a further embodiment of the present invention, nucleotide sequences encoding interferon-gamma (IFNγ) are also provided. The IFNγ may be provided in a separate nucleotide sequence to any of components (i), (ii) or (iii). In an embodiment of the invention in which component (i) is a nucleotide sequence encoding a TLR agonist, the IFNγ may be co-encoded within a nucleotide sequence encoding one or more of components (i), (ii) or (iii). Any remaining components may be encoded within separate nucleotide sequences, or may be co-encoded within a single further nucleotide sequence.

In one embodiment, the IFNγ is encoded within a nucleotide sequence encoding components (ii) and (iii), or components (ii) and (iii) and the IFNγ are encoded within the same or separate plasmid molecules, and component (i) is provided in a separate composition for concomitant or sequential administration. For example components (ii) and (iii) and the IFNγ are encoded within separate plasmid molecules. In one embodiment, component (i) may be an imidazoqulnoline molecule, or derivative thereof, for example imiquimod.

In a further embodiment of the present invention, nucleotide sequences encoding CD40 ligand (CD40L) are also provided. The CD40L may be provided in a separate nucleotide sequence to any of components (i), (ii) or (iii). In an embodiment of the invention in which component (i) is a nucleotide sequence encoding a TLR agonist, the CD40L may be co-encoded within a nucleotide sequence encoding one or more of components (I), (ii) or (iii). Any remaining components may be encoded within separate nucleotide sequences, or may be co-encoded within a single further nucleotide sequence.

In one embodiment, the CD40L is encoded within a nucleotide sequence encoding components (ii) and (iii), or components (ii) and (iii) and the CD40L are encoded within the same or separate plasmid molecules, and component (i) is provided in a separate composition for concomitant or sequential administration. For example components (ii) and (iii) and the CD40L are encoded within separate plasmid molecules. In one embodiment, component (i), may be an imidazoquinoline molecule, or derivative thereof, for example imiquimod.

All nucleotide sequences referred to herein may be RNA or DNA sequences. Further, all nucleotide sequences may be comprised or consist within plasmid DNA.

In an embodiment where components (ii) and (iii) are provided for concomitant administration, plasmids comprising nucleotide sequences encoding components (ii) and (iii) may be delivered to the same cell, or to neighbouring cells. In one embodiment, where the plasmids are delivered to neighbouring cells, expression causes release of components into the same micro-environment. In one embodiment, component (i) is provided in a separate composition for concomitant or sequential delivery. In a further embodiment delivery is concomitant. In an alternative embodiment, component (i) is provided in a separate composition for delivery 12 hours or 24 hours after delivery of components (ii) and (iii). Delivery of component (I) may be at the same site as delivery of components (ii) and (iii). By same site is meant component (i) may be delivered within 15 cm of the delivery site, within 5 cm, within 1 cm, or may be at the injection site of components (ii) and (iii).

In an alternative embodiment of the present invention, one or more components may be administered at different injection sites. In one embodiment, components are all administered at sites which all drain into the same lymph node or nodes.

In one embodiment of the present invention, the nucleotide sequence encoding (iil) encodes a MUC-1 protein or derivative which is capable of raising an immune response in vivo, the immune response being capable of recognising a MUC-1 expressing tumour cell or tumour.

In a further embodiment of the present invention, the nucleotide sequence encoding (iii) encodes a P501S protein or derivative which is capable of raising an immune response in vivo, the immune response being capable of recognising a P501S expressing tumour cell or tumour.

The present invention further proves a vaccine composition comprising an immunogenic composition or compositions according to the present invention, and pharmaceutically acceptable carrier(s), diluent(s) or excipient(s)

The present invention further provides a process for the manufacture of an immunogenic composition comprising mixing adjuvant components (i) and (ii) of the present invention with an immunogen component (iii) comprising a nucleotide sequence encoding an antigenic peptide or protein. In one embodiment the process comprises mixing the nucleotide molecule encoding adjuvant component (ii) with nucleotide encoding the immunogen component (iii), and providing adjuvant component (i) or a nucleotide sequence encoding adjuvant component (i) in a separate composition for concomitant or sequential administration. Alternatively, the process comprising co-encoding the nucleotide molecule encoding adjuvant component (ii) with nucleotide encoding the immunogen component (iii) to form a single polynucleotide molecule, and providing adjuvant component (i) or a nucleotide sequence encoding adjuvant component (i) in a separate composition for concomitant or sequential administration

In an alternative embodiment, there is provided a process in which nucleotide sequences encoding components (i), (ii) and (iii) are encoded within separate polynucleotide molecules, for concomitant or sequential administration. In a yet further embodiment, there is provided a process in which the nucleotide sequences encoding any two of components (i), (ii) and (iii) are co-encoded to form a single polynucleotide molecule, and the remaining nucleotide sequence is encoded within a further polynucleotide sequence for concomitant or sequential administration. Alternatively nucleotide sequences encoding components (i), (ii) and (iii) are co-encoded to form a single polynucleotide molecule

In one embodiment, the nucleotide sequence used in the process is DNA, and the nucleotide sequence which may be used in the process is encoded within plasmid DNA

In an alternative embodiment, there is provided a process in which the nucleotide molecules encoding components (ii) and (iii) are incorporated within a plasmid, and adjuvant component (i) is provided in a separate composition for concomitant or sequential administration.

In an further embodiment, the process further provides incorporating the components within pharmaceutically acceptable excipients, diluents or carriers.

The invention further provides a pharmaceutical composition or compositions comprising adjuvant components (i) and (ii) according to the present invention; an immunogen component (iii) comprising a nucleotide sequence encoding an antigenic peptide or protein; and one or more pharmaceutically acceptable excipients, diluents or carriers.

Alternatively, the present invention provides a pharmaceutical composition or compositions comprising an immunogenic composition or compositions as described herein, and pharmaceutically acceptable excipients, diluents or carriers

The present invention further provides a kit comprising a pharmaceutical composition comprising adjuvant component (ii); immunogen component (iii), and a pharmaceutical acceptable excipient, diluent or carrier; and a further pharmaceutical composition comprising adjuvant component (i), and a pharmaceutical acceptable excipient, diluent or carrier, in which: adjuvant component (i) comprises a TLR agonist, or a nucleotide encoding a TLR agonist; adjuvant component (ii) comprises a nucleotide encoding GM-CSF; and immunogen component (iii) comprises a nucleotide sequence encoding an antigenic peptide or protein. In one embodiment, at least one carrier is a gold bead and at least one pharmaceutical composition is amenable to delivery by particle mediated drug delivery. In a further embodiment the carrier for components (ii) and (iii) is a gold bead and adjuvant component (i) is formulated for concomitant or sequential administration. In one aspect of the present invention there is provided a method comprising packaging nucleotide sequences encoding one or more of components (ii) and (iii) onto gold beads. In one embodiment of the present invention, components are packaged onto separate populations of gold beads which are then combined before administration. In an alternative embodiment, components are packaged onto the same population of gold beads. In a further embodiment, components (ii) and (iii) are packaged onto gold beads, and component (i) is provided in a separate composition for concomitant or sequential administration.

The present invention further provides a method of treating a patient suffering from or susceptible to a tumour, by the administration of a safe and effective amount of an immunogenic, vaccine or pharmaceutical composition as herein described. In one embodiment the tumour to be treated is a MUC-1 or P501S expressing tumour. The tumour to be treated may be carcinoma of the breast; carcinoma of the lung, including non-small cell lung carcinoma; or prostate, gastric and other gastrointestinal carcinomas

The present invention further provides a method of increasing an immune response of a mammal to an antigen, the method comprising administration to the mammal the following components:

(i) a TLR agonist, or a nucleotide encoding a TLR agonist;
(ii) a nucleotide encoding GM-CSF; and
(iii) an immunogen component comprising a nucleotide sequence encoding an antigenic peptide or protein

In one embodiment, the method comprises concomitant administration of any two of components (i), (ii) and (iii), and sequential administration of the remaining component. Alternatively, the method comprises sequential administration of components (i), (ii) and (iii). In a further embodiment, the components for concomitant administration are formulated into separate compositions. In one method. of the present invention, components (ii) and (iii) are administered concomitantly, and component (i) is provided in a separate composition for concomitant or sequential administration. In one embodiment, component (i) is an imidazoquinoline or derivative thereof. Component (i) may be imiquimod, and may be provided in the form of Aldara™ cream (3M) for topical administration at or near the site of administration of components (ii) and (iii).

The present invention further provides an immunogenic composition comprising the following components, in the manufacture of a medicament for use in the treatment or prophylaxis of MUC-1 or P501S expressing tumours:

(i) a TLR agonist, or a nucleotide encoding a TLR agonist;
(ii) a nucleotide encoding GM-CSF; and
(iii) an immunogen component comprising a nucleotide sequence encoding a MUC-1 or P501 antigenic peptide or protein.

The present invention further provides a method of raising an immune response in a mammal against a disease state, comprising administering to the mammal within an appropriate vector, a nucleotide sequence encoding an antigenic peptide associated with the disease state; additionally administering to the mammal within an appropriate vector, a nucleotide sequence encoding GM-CSF; and further administering to the mammal an imidazoquinoline or derivative thereof to raise the immune response.

The present invention further provides a method of increasing the immune response of a mammal to an immunogen, comprising the step of administering to the mammal within an appropriate vector, a nucleotide sequence encoding the immunogen in an amount effective to stimulate an immune response and a nucleotide sequence encoding GM-CSF; and further administering to the mammal an imidazoquinoline or derivative thereof in an amount effective to increase the immune response.

The present invention further provides a method of administration of any of the compositions as herein described.

The present invention further provides use of an imidazoquinoline or derivative thereof and GM-CSF in the manufacture of a medicament for enhancing immune responses initiated by an antigenic peptide or protein, the antigenic peptide or protein being expressed as a result of administration to a mammal of a nucleotide sequence encoding for the peptide.

The present invention further provides the use of the following components (i) to (iii) in the manufacture of a medicament for the enhancement of an immune response to an antigen encoded by a nucleotide sequence:

(i) a TLR agonist, or a nucleotide encoding a TLR agonist;
(ii) a nucleotide encoding GM-CSF; and
(iii) an immunogen component comprising a nucleotide sequence encoding an antigenic peptide or protein

The present invention further provides the use of the following components (i) to (iii) in the manufacture of two or more medicaments for concomitant or sequential administration to a mammal for the enhancement of an immune response to an antigen encoded by a nucleotide sequence:

(i) a TLR agonist, or a nucleotide encoding a TLR agonist;
(ii) a nucleotide encoding GM-CSF; and
(iii) an immunogen component comprising a nucleotide sequence encoding an antigenic peptide or protein

The present invention further provides the use of the following components (i) to (iii) in the manufacture of medicaments for concomitant or sequential administration to a mammal for the enhancement of an immune response to an antigen encoded by a nucleotide sequence, in which each component is formulated into a separate medicament:

(i) a TLR agonist, or a nucleotide encoding a TLR agonist;
(ii) a nucleotide encoding GM-CSF; and
(iii) an immunogen component comprising a nucleotide sequence encoding an antigenic peptide or protein

The adjuvant composition or compositions described herein may be used at the “prime” and/or “boost” stage of a “prime-only” strategy, or in a “prime-boost” approach. The “prime-boost” approach used may comprise two nucleic acid vaccines, or may comprise two distinct vaccine preparations (one nucleic acid, one protein). An example of the “prime-boost” approach is described in Barnett et al., Vaccine 15:869-873 (1997), where two distinct vaccine preparations (one DNA, one protein) are prepared and administered separately, at different times, and in a specific order.

In one embodiment, compositions as described herein are used at the “prime” stage of a vaccination strategy.

### DETAILED DESCRIPTION OF THE INVENTION

Throughout this specification and the appended claims, unless the context requires otherwise, the words “comprise” and “include” or variations such as “comprising”, “comprises”, “including”, “includes”, etc., are to be construed inclusively, that is, use of these words will imply the possible inclusion of integers or elements not specifically recited. Additionally, the terms ‘comprising’, ‘comprise’ and ‘comprises’ herein is intended to be optionally substitutable by the terms ‘consisting of’, ‘consist of’ and ‘consists of’, respectively, in every instance.

Additionally, throughout this specification and the appended claims, except in relation to the experimental data, examples and figures, the term “GM-CSF” is optionally substitutable by the term “IFNγ”, and vice-versa, in every instance. In one embodiment of the present invention, where component (ii) is a nucleotide sequence encoding IFNγ, component (i) may be a TLR agonist of TLR-2,4,7 or 8.

As described above, the present invention relates to immunogenic compositions, vaccine compositions, vaccination methods, and to improvements of methods of vaccination involving the introduction into a mammal of nucleotide sequence which encodes for an immunogen which is an antigenic protein or peptide, such that the protein or peptide will be expressed within the mammalian body to thereby induce an immune response within the mammal against the antigenic protein or peptide. Such methods of vaccination are well known and are fully described in Donnelly et al and Ertl et al as referred to above.

As used herein the term immunogenic composition refers to a combination of

(i) a TLR agonist, or nucleotide sequence encoding a TLR agonist;
(ii) a nucleotide sequence encoding GM-CSF; and
(iii) an immunogen component comprising a nucleotide sequence encoding an antigenic peptide or protein
in which components (i) and (ii) act in functional co-operation to enhance the immune responses in a mammal to the immunogen component (iii).

The combination is, for example, in the form of an admixture of the three components in a single pharmaceutically acceptable formulation or in the form of separate, individual components, for example in the form of a kit comprising adjuvant components (i) and (ii) and immunogen component (iii) wherein the three components are for separate, sequential or simultaneous administration. In one embodiment, the administration of the three components is concomitant. In a further embodiment of the present invention, components (ii) and (iii) are administered concomitantly, and component (i) is administered separately, prior to administration of components (ii) and (iii). In a further embodiment of the present invention, components (ii) and (iii) are administered concomitantly, and component (i) is administered separately, after administration of components (ii) and (iii).

The imidazoquinoline or derivative thereof as referred to throughout the specification and the claims may be a compound defined by one of Formulae I-VI below:

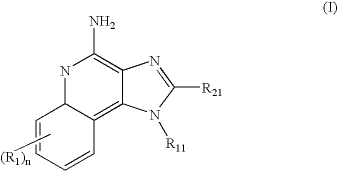

wherein
R₁₁ is selected from the group consisting of straight or branched chain alkyl, hydroxyalkyl, acyloxyalkyl, benzyl, (phenyl)ethyl and phenyl, the benzyl, (phenyl)ethyl or phenyl substituent being optionally substituted on the benzene ring by one or two moieties independently selected from the group consisting of alkyl of one to about four carbon atoms, alkoxy of one to about four carbon atoms and halogen, with the proviso that if the benzene ring is substituted by two of the moieties, then the moieties together contain no more than 6 carbon atoms; R₂₁ is selected from the group consisting of hydrogen, alkyl of one to about eight carbon atoms, benzyl, (phenyl)ethyl and phenyl, the benzyl, (phenyl)ethyl or phenyl substituent being optionally substituted on the benzene ring by one or two moieties independently selected from the group consisting of alkyl of one to about four carbon atoms, alkoxy of one to about four carbon atoms and halogen, with the proviso that when the benzene ring is substituted by two of the moieties, then the moieties together contain no more than 6 carbon atoms; and each R₁ is independently selected from the group consisting of hydrogen, alkoxy of one to about four carbon atoms, halogen and alkyl of one to about four carbon atoms, and n is an integer from 0 to 2, with the proviso that if n is 2, then the R₁ groups together contain no more than 6 carbon atoms;

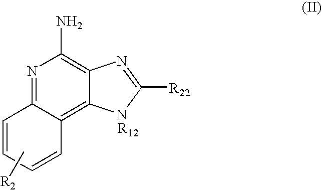

wherein
R₁₂ is selected from the group consisting of straight chain or branched chain alkenyl containing 2 to about 10 carbon atoms and substituted straight chain or branched chain alkenyl containing 2 to about 10 carbon atoms, wherein the substituent is selected from the group consisting of straight chain or branched chain alkyl containing 1 to about 4 carbon atoms and cycloalkyl containing 3 to about 6 carbon atoms; and cycloalkyl containing 3 to about 6 carbon atoms substituted by straight chain or branched chain alkyl containing 1 to about 4 carbon atoms; and R₂₂ is selected from the group consisting of hydrogen, straight chain or branched chain alkyl containing one to about eight carbon atoms, benzyl, (phenyl)ethyl and phenyl, the benzyl, (phenyl)ethyl or phenyl substituent being optionally substituted on the benzene ring by one or two moieties independently selected from the group consisting of straight chain or branched chain alkyl containing one to about four carbon atoms, straight chain or branched chain alkoxy containing one to about four carbon atoms, and halogen, with the proviso that when the benzene ring is substituted by two such moieties, then the moieties together contain no more than 6 carbon atoms; and each R₂ is independently selected from the group consisting of straight chain or branched chain alkoxy containing one to about four carbon atoms, halogen, and straight chain or branched chain alkyl containing one to about four carbon atoms, and n is an integer from zero to 2, with the proviso that if n is 2, then the R₂ groups together contain no more than 6 carbon atoms;

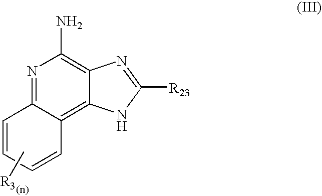

wherein
R₂₃ is selected from the group consisting of hydrogen, straight chain or branched chain alkyl of one to about eight carbon atoms, benzyl, (phenyl)ethyl and phenyl, the benzyl, (phenyl)ethyl or phenyl substituent being optionally substituted on the benzene ring by one or two moieties independently selected from the group consisting of straight chain or branched chain alkyl of one to about four carbon atoms, straight chain or branched chain alkoxy of one to about four carbon atoms, and halogen, with the proviso that when the benzene ring is substituted by two such moieties, then the moieties together—contain no more than 6 carbon atoms; and each R₅ is independently selected from the group consisting of straight chain or branched chain alkoxy of one to about four-carbon atoms, halogen, and 30 straight chain or branched chain alkyl of one to about four carbon atoms, and n is an integer from zero to 2, with the proviso that if n is 2, then the R₃ groups together contain no more than 6 carbon atoms;

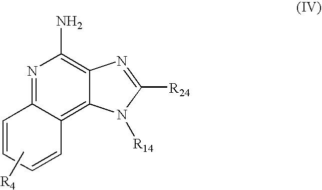

wherein
R₁₄ is —CHR_{A}R_{B} wherein R_{B} is hydrogen or a carbon-carbon bond, with the proviso that when R_{B} is hydrogen R_{A} is alkoxy of one to about four carbon atoms, hydroxyalkoxy of one to about four carbon atoms, 1-alkynyl of two to about ten carbon atoms, tetrahydropyranyl, alkoxyalkyl wherein the alkoxy moiety contains one to about four carbon atoms and the alkyl moiety contains one to about four carbon atoms, 2-, 3-, or 4-pyridyl, and with the further proviso that when R_{B} is a carbon-carbon bond R_{B} and R_{A} together form a tetrahydrofuranyl group optionally substituted with one or more substituents independently selected from the group consisting of hydroxy and hydroxyalkyl of one to about four carbon atoms; R₂₄ is selected from the group consisting of hydrogen, alkyl of one to about four carbon atoms, phenyl, and substituted phenyl wherein the substituent is selected from the group consisting of alkyl of one to about four carbon atoms, alkoxy of one to about four carbon atoms, and halogen; and R₄ is selected from the group consisting of hydrogen, straight chain or branched chain alkoxy containing one to about four carbon atoms, halogen, and straight chain or branched chain alkyl containing one to about four carbon atoms;

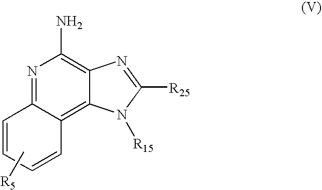

wherein
R₁₅ is selected from the group consisting of: hydrogen; straight chain or branched chain alkyl containing one to about ten carbon atoms and substituted straight chain or branched chain alkyl containing one to about ten carbon atoms, wherein the substituent is selected from the group consisting of cycloalkyl containing three to about six carbon atoms and cycloalkyl containing three to about six carbon atoms substituted by straight chain or branched chain alkyl containing one to about four carbon atoms; straight chain or branched chain alkenyl containing two to about ten carbon atoms and substituted straight chain or branched chain alkenyl containing two to about ten carbon atoms, wherein the substituent is selected from the group consisting of cycloalkyl containing three to about six carbon atoms and cycloalkyl containing three to about six carbon atoms substituted by straight chain or branched chain alkyl containing one to about four carbon atoms; hydroxyalkyl of one to about six carbon atoms; alkoxyalkyl wherein the alkoxy moiety contains one to about four carbon atoms and the alkyl moiety contains one to about six carbon atoms; acyloxyalkyl wherein the acyloxy moiety is alkanoyloxy of two to about four carbon atoms or benzoyloxy, and the alkyl moiety contains one to about six carbon atoms; benzyl; (phenyl)ethyl; and phenyl; the benzyl, (phenyl)ethyl or phenyl substituent being optionally substituted on the benzene ring by one or two moieties independently selected from the group consisting of alkyl of one to about four carbon atoms, alkoxy of one to about four carbon atoms, and halogen, with the proviso that when the benzene ring is substituted by two of the moieties, then the moieties together contain no more than six carbon atoms;

### R₂₅ is

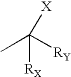

wherein
R_{X} and R_{Y} are independently selected from the group consisting of hydrogen, alkyl of one to about four carbon atoms, phenyl, and substituted phenyl wherein the substituent is elected from the group consisting of alkyl of one to about four carbon atoms, alkoxy of one to about four carbon atoms, and halogen; X is selected from the group consisting of alkoxy containing one to about four carbon atoms, alkoxyalkyl wherein the alkoxy moiety contains one to about four carbon atoms and the alkyl moiety contains one to about four carbon atoms, haloalkyl of one to about four carbon atoms, alkylamido wherein the alkyl group contains one to about four carbon atoms, amino, substituted amino wherein the substituent is alkyl or hydroxyalkyl of one to about four carbon atoms, azido, alkylthio of one to about four carbon atoms; and R₅ is selected from the group consisting of hydrogen, straight chain or branched chain alkoxy containing one to about four carbon atoms, halogen, and straight chain or branched chain alkyl containing one to about four carbon atoms; or a pharmaceutically acceptable salt of any of the foregoing.

Alkyl groups may be C₁-C₄ alkyl, for example methyl, ethyl, propyl, 2-methylpropyl and butyl. Alkyl groups may be methyl, ethyl and 2-methyl-propyl. Alkoxy groups may be methoxy, ethoxy and ethoxymethyl.

The compounds recited above and methods for their preparation are disclosed in PCT patent application publication number WO 94/17043.

In instances where n can be zero, one, or two, n may be zero or one.

The substituents R₁-R₅ above are generally designated “benzo substituents” herein. The benzo substituent may be hydrogen.

The substituents R₁₁-R₁₅ above are generally designated “1-substituents” herein. The 1-substituent may be 2-methylpropyl or 2-hydroxy-2-methylpropyl.

The substituents R₂₁-R₂₅ above are generally designated “2-substituents”, herein. The 2-substituents may be hydrogen, alkyl of one to about six carbon atoms, alkoxyalkyl wherein the alkoxy moiety contains one to about four carbon atoms and the alkyl moiety contains one to about four carbon atoms. The 2-substituent may be hydrogen, methyl, or ethoxymethyl.

The 1H-imidazo[4,5-c]quinolin-4-amine may be a compound defined by formula VI below:

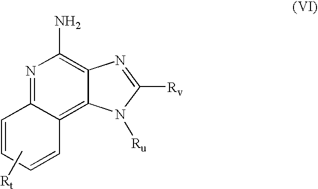

### Wherein

Rₜ is selected from the group consisting of hydrogen, straight chain or branched chain alkoxy containing one to about four carbon atoms, halogen, and straight chain or branched chain alkyl containing one to about four carbon atoms;
Rᵤ is 2-methylpropyl or 2-hydroxy-2-methylpropyl; and
Rᵥ is hydrogen, alkyl of one to about six carbon atoms, or alkoxyalkyl wherein the alkoxy moiety contains one to about four carbon atoms and the alkyl moiety contains one to about four carbon atoms; or physiologically acceptable salts of any of the foregoing, where appropriate.

In formula VI, Rₜ may be hydrogen, Rᵤ may be 2-methylpropyl or 2-hydroxy-2-methylpropyl, and Rv may be hydrogen, methyl or ethoxymethyl.

1H-imidazo[4,5-c]quinolin-4-amines may include the following:

1-(2-methylpropyl)-1H-imidazo[4,5-c]quinolin-4-amine (a compound of formula VI wherein Rₜ is hydrogen, Rᵤ is 2-methylpropyl and Rᵥ is hydrogen);
1-(2-hydroxy-2-methylpropyl)-2-methyl-1H-imidazo[4,5-c]quinolin-4-amine (a compound of formula VI wherein Rₜ is hydrogen, Rᵤ is 2-hydroxy-2-methylpropyl, and Rᵥ is methyl;
1-(2-hydroxy-2-methylpropyl)-1H-imidazo[4,5-c]quinolin-4-amine (a compound of formula VI wherein Rₜ is hydrogen, Rᵤ is 2-hydroxy-2-methylpropyl, and Rᵥ is hydrogen)
1-(2-hydroxy-2-methylpropyl)-2-ethoxymethyl-1-H-imidazo[4,5-c]quinolin-4-amine (a compound of formula VI wherein Rₜ is hydrogen, Rᵤ is 2-hydroxy-2-methylpropyl and Rᵥ is ethoxymethyl);
or physiologically acceptable salts thereof.

### Disease States

It is possible for the vaccination methods and compositions according to the present application to be adapted for protection or treatment of mammals against a variety of disease states such as, for example, viral, bacterial or parasitic infections, cancer, allergies and autoimmune disorders. Some specific examples of disorders or disease states which can be protected against or treated by using the methods or compositions according to the present invention, are as follows:

### Viral Infections

Hepatitis viruses A, B, C, D & E, HIV, herpes viruses 1, 2, 6 & 7,-cytomegalovirus, varicella zoster, papilloma virus, Epstein Barr virus, influenza viruses, para-influenza viruses, adenoviruses, coxsakie viruses, picorna viruses, rotaviruses, respiratory syncytial viruses, pox viruses, rhinoviruses, rubella virus, papovirus, mumps virus, measles virus.

### Bacterial Infections

Mycobacteria causing TB and leprosy, pneumocci, aerobic gram negative bacilli, mycoplasma, staphyloccocal infections, streptococcal infections, salmonellae, chlamydiae.

### Parasitic

Malaria, leishmaniasis, trypanosomiasis, toxoplasmosis, schistosomiasis, filariasis,

### Cancer

Breast cancer, colon cancer, rectal cancer, cancer of the head and neck, renal cancer, malignant melanoma, laryngeal cancer, ovarian cancer, cervical cancer, prostate cancer.

### Allergies

Rhinitis due to house dust mite, pollen and other environmental allergens

### Autoimmune Disease

Systemic lupus erythematosis

In one embodiment, the methods or compositions of the present invention are used to protect against or treat the viral disorders Hepatitis B, Hepatitis C, Human papilloma virus, Human immunodeficiency virus, or Herpes simplex virus; the bacterial disease TB; cancers of the breast, colon, ovary, cervix, and prostate; or the autoimmune diseases of asthma, rheumatoid arthritis and Alzheimer's

It is to be recognised that these specific disease states have been referred to by way of example only, and are not intended to be limiting upon the scope of the present invention.

### Antigen or Immunogen

The nucleotide sequences of component (iii) referred to in this application, encoding antigen or immunogen to be expressed within a mammalian system, in order to induce an antigenic response, may encode for an entire protein, or merely a shorter peptide sequence which is capable of initiating an antigenic response. Throughout this specification and the appended claims, the phrase “antigenic peptide” or “immunogen” is intended to encompass all peptide or protein sequences which are capable of inducing an immune response within the animal concerned. In one embodiment, however, the nucleotide sequence will encode for a full protein which is associated with the disease state, as the expression of full proteins within the animal system are more likely to mimic natural antigen presentation, and thereby evoke a full immune response. Some non-limiting examples of known antigenic peptides in relation to specific disease states include the following:

Antigens which are capable of eliciting an immune response against a human pathogen, which antigen or antigenic composition is derived from HIV-1, (such as tat, nef, gp120 or gp160, gp40, p24, gag, env, vif, vpr, vpu, rev), human herpes viruses, such as gH, gL gM gB gC gK gE or gD or derivatives thereof or Immediate Early protein such as ICP27, ICP 47, IC P 4, ICP36 from HSV1 or HSV2, cytomegalovirus, especially Human, (such as gB or derivatives thereof), Epstein Barr virus (such as gp350 or derivatives thereof), Varicella Zoster Virus (such as gpI, II, III and IE63), or from a hepatitis virus such as hepatitis B virus (for example Hepatitis B Surface antigen or Hepatitis core antigen or pol), hepatitis C virus antigen and hepatitis E virus antigen, or from other viral pathogens, such as paramyxoviruses: Respiratory Syncytial virus (such as F and G proteins or derivatives thereof), or antigens from parainfluenza virus, measles virus, mumps virus, human papilloma viruses (for example HPV6, 11, 16, 18, eg L1, L2, E1, E2, E3, E4, E5, E6, E7), flaviviruses (e.g. Yellow Fever Virus, Dengue Virus, Tick-borne encephalitis virus, Japanese Encephalitis Virus) or Influenza virus cells, such as HA, NP, NA, or M proteins, or combinations thereof), or antigens derived from bacterial pathogens such as Neisseria spp, including N. gonorrhea and N. meningitidis, eg, transferrin-binding proteins, lactoferrin binding proteins, PilC, adhesins); S. pyogenes (for example M proteins or fragments thereof, C5A protease, S. agalactiae, S. mutans; H. ducreyi; Moraxella spp, including M catarrhalis, also known as Branhamella catarrhalis (for example high and low molecular weight adhesins and invasins); Bordetella spp, including B. pertussis (for example pertactin, pertussis toxin or derivatives thereof, filamenteous hemagglutinin, adenylate cyclase, fimbriae), B. parapertussis and B. bronchiseptica; Mycobacterium spp., including M. tuberculosis (for example ESAT6, Antigen 85A, -B or -C, MPT 44, MPT59, MPT45, HSP10,HSP65, HSP70, HSP 75, HSP90, PPD 19 kDa [Rv3763], PPD 38 kDa [Rv0934]), M. bovis, M. leprae, M. avium, M. paratuberculosis, M. smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli (for example shiga toxin-like toxin or derivatives thereof; Vibrio spp, including V. cholera (for example cholera toxin or derivatives thereof); Shigella spp, including S. sonnei, S. dysenteriae, S. flexnerii; Yersinia spp, including Y. enterocolitica (for example a Yop protein), Y. pestis, Y. pseudotuberculosis; Campylobacter spp, including C. jejuni (for example toxins, adhesins and invasins) and C. coli; Salmonella spp, including S. typhi, S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp., including C. trachomatis (for example MOMP, heparin-binding proteins), C. pneumoniae (for example MOMP, heparin-binding proteins), C. psittaci; Leptospira spp., including L. interrogans; Treponema spp., including T. pallidum (for example the rare outer membrane proteins), T. denticola, T. hyodysenteriae; or derived from parasites such as Plasmodium spp., including P. falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; leishmania spp., including L. major; Pneumocystis spp., including P. carinii; Trichomonas spp., including T. vaginalis; Schisostoma spp., including S. mansoni, or derived from yeast such as Candida spp., including C. albicans; Cryptococcus spp., including C. neoformans.

Other specific antigens for M. tuberculosis include for example Rv2557, Rv2558, RPFs: Rv0837c, Rv1884c, Rv2389c, Rv2450, Rv1009, aceA (Rv0467), PstS1, (Rv0932), SodA (Rv3846), Rv2031c 16 kDal., Tb Ra12, Tb H9, Tb Ra35, Tb38-1, Erd 14, DPV, MTI, MSL, mTTC2 and hTCC1 (WO 99/51748). Proteins for M. tuberculosis also include fusion proteins and variants thereof where at least two, or three polypeptides of M. tuberculosis are fused into a larger protein. Fusions include Ra12-TbH9-Ra35, Erd14-DPV-MTI, DPV-MTI-MSL, Erd 14-DPV-MTI-MSL-mTCC2, Erd 14-DPV-MTI-MSL, DPV-MTI-MSL-mTCC2, TbH9-DPV-MTI (WO 99/51748).

In one embodiment antigens for Chlamydia include for example the High Molecular Weight Protein (HWMP) (WO 99/17741), ORF3 (EP 366 412), and putative membrane proteins (Pmps). Other Chlamydia antigens of the vaccine formulation can be selected from the group described in WO 99/28475.

In one embodiment bacterial vaccines comprise antigens derived from Streptococcus spp, including S. pneumoniae (PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (WO 90/06951; WO 99/03884). Other bacterial vaccines comprise antigens derived from Haemophilus spp., including H. influenzae type B (for example PRP and conjugates thereof), non typeable H. influenzae, for example OMP26, high molecular weight adhesins, P5, P6, protein D and lipoprotein D, and fimbrin and fimbrin derived peptides (U.S. Pat. No. 5,843,464) or multiple copy variants or fusion proteins thereof.

The antigens that may be used in the present invention may further comprise antigens derived from parasites that cause Malaria. For example, antigens from Plasmodia falciparum include RTS,S and TRAP. RTS is a hybrid protein comprising substantially all the C-terminal portion of the circumsporozoite (CS) protein of P. falciparum linked via four amino acids of the preS2 portion of Hepatitis B surface antigen to the surface (S) antigen of hepatitis B virus. Its full structure is disclosed in the International Patent Application No.

PCT/EP92/02591, published under Number WO 93/10152 claiming priority from UK patent application No. 9124390.7. When expressed in yeast RTS is produced as a lipoprotein particle, and when it is co-expressed with the S antigen from HBV it produces a mixed particle known as RTS,S. TRAP antigens are described in the International Patent Application No. PCT/GB89/00895, published under WO 90/01496. An embodiment of the present invention is a Malaria vaccine wherein the antigenic preparation comprises a combination of the RTS, S and TRAP antigens. Other plasmodia antigens that are likely candidates to be components of a multistage Malaria vaccine are P. faciparum MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in Plasmodium spp.

The invention contemplates the use of an anti-tumour antigen and be useful for the immunotherapeutic treatment of cancers. For example, tumour rejection antigens such as those for prostrate, breast, colorectal, lung, pancreatic, renal or melanoma cancers. Exemplary antigens include MAGE 1, 3 and MAGE 4 or other MAGE antigens such as disclosed in WO99/40188, PRAME, BAGE, Lage (also known as NY Eos 1) SAGE and HAGE (WO 99/53061) or GAGE (Robbins and Kawakami, 1996, Current Opinions in Immunology 8, pps 628-636; Van den Eynde et al., International Journal of Clinical & Laboratory Research (submitted 1997); Correale et al. (1997), Journal of the National Cancer Institute 89, p293. Indeed these antigens are expressed in a wide range of tumour types such as melanoma, lung carcinoma, sarcoma and bladder carcinoma.

MAGE antigens for use in the present. invention may be expressed as a fusion protein with an expression enhancer or an Immunological fusion partner. In particular, the Mage protein may be fused to Protein D from Heamophilus influenzae B. In particular, the fusion partner may comprise the first ⅓ of Protein D. Such constructs are disclosed in WO99/40188. Other examples of fusion proteins that may contain cancer specific epitopes include bcr/abl fusion proteins.

In one embodiment prostate antigens are utilised, such as Prostate specific antigen (PSA), PAP, PSCA (PNAS 95(4) 1735-1740 1998), PSMA or antigen known as Prostase.

Prostase is a prostate-specific serine protease (trypsin-like), 254 amino acid-long, with a conserved serine protease catalytic triad H-D-S and a amino-terminal pre-propeptide sequence, indicating a potential secretory function (P. Nelson, Lu Gan, C. Ferguson, P. Moss, R. Gelinas, L. Hood & K. Wand, “Molecular cloning and characterisation of prostase, an androgen-regulated serine protease with prostate restricted expression, In Proc. Natl. Acad. Sci. USA (1999) 96, 3114-3119). A putative glycosylation site has been described. The predicted structure is very similar to other known serine proteases, showing that the mature polypeptide folds into a single domain. The mature protein is 224 amino acids-long, with one A2 epitope shown to be naturally processed.

Prostase nucleotide sequence and deduced polypeptide sequence and homologs are disclosed in Ferguson, et al. (Proc. Natl. Acad. Sci. USA 1999, 96, 3114-3119) and in International Patent Applications No. WO 98/12302 (and also the corresponding granted U.S. Pat. No. 5,955,306), WO 98/20117 (and also the corresponding granted U.S. Pat. No. 5,840,871 and U.S. Pat. No. 5,786,148) (prostate-specific kallikrein) and WO 00/04149 (P703P).

The present invention provides antigens comprising prostase protein fusions based on prostase protein and fragments and homologues thereof (“derivatives”). Such derivatives are suitable for use in therapeutic vaccine formulations which are suitable for the treatment of a prostate tumours. Typically the fragment will contain at least 20, 50, or 100 contiguous amino acids as disclosed in the above referenced patent and patent applications.

A further prostate antigen is known as P501S, sequence ID no 113 of WO98/37814, incorporated herein by reference. P501S is a membrane protein which interacts with a cell surface receptor. It is predicted to be a type 111a plasma membrane protein with 9-11 transmembrane regions spanning the whole length of the protein. P501S shares some homologies with spinach sucrose binding protein (Riesmeier J W, Willmitzer L, Frommer W B, 1992, EMBO J. 11, 4705-13).

Contiguous and partially overlapping P501S cDNA fragments and polypeptides encoded thereby, have also been described (WO 98/50567), more particularly a C-terminal fragment of 255 amino acids in length. A polypeptide of 231 amino acids in length, described in WO 99/67384, is reported to comprise a potential transmembrane domain, two potential caseine kinase 11 phosphorylation sites, one potential protein kinase C phosphorylation site and a potential cell attachment sequence.

P501S and constructs thereof are also described in U.S. Pat. No. 6,329,505 also incorporated herein by reference. Immunogenic fragments and portions encoded by the gene thereof comprising at least 20, 50, or 100 contiguous amino acids as disclosed in the above referenced patent application, are contemplated. A particular fragment is PS108 (WO 98/50567, incorporated herein by reference).

Other prostate specific antigens are known from Wo98/37418, and WO/004149. Another is STEAP PNAS 96 14523 14528 7-12 1999.

Other tumour associated antigens useful in the context of the present invention include: Plu-1 J. Biol. Chem. 274 (22) 15633-15645, 1999, HASH-1, HasH-2, Cripto (Salomon et al Bioessays 199, 21 61-70, U.S. Pat. No. 5,654,140) Criptin U.S. Pat. No. 5,981,215, . . . , Additionally, antigens particularly relevant for vaccines in the therapy of cancer also comprise tyrosinase and survivin.

The present invention is also useful in combination with breast cancer antigens such as Muc-1, Muc-2, EpCAM, her 2/Neu, mammaglobin (U.S. Pat. No. 5,668,267) or those disclosed in WO00/52165, WO99/33869, WO99/19479, WO98/45328.

The epithelial cell mucin MUC-1 (also known as episialin or polymorphic epithelial mucin, PEM) is a large molecular-weight glycoprotein expressed on many epithelial cells, which has been described in WO01/46228 and WO03/100060.

In one embodiment, component (iii) encodes a MUC-1 protein or derivative which is devoid of any repeat units (perfect or imperfect). In a further embodiment, the MUC-1 protein or derivative is devoid of only the perfect repeat units. In yet a further embodiment the MUC-1 protein or derivative contains between one and 15 repeat units; 7 perfect repeat units

In an embodiment of the invention, the MUC-1 derivative may be codon-modified from wild-type Muc-1. In particular, the non-perfect repeat region may have a RSCU (Relative Synonymous Codon Usage) of at least 0.6, or at least 0.65. The nucleotide sequence encoding the non-perfect repeat units of the MUC-1 protein or derivative may have a level of identity with respect to wild-type MUC-1 DNA over the corresponding non-repeat regions of less than 85%, or of less than 80%. The DNA code has 4 letters (A, T, C and G) and uses these to spell three letter “codons” which represent the amino acids the proteins encodes in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons.

Where more than one codon is available to code for a given amino acid, it has been observed that the codon usage patterns of organisms are highly non-random. Different species show a different bias in their codon selection and, furthermore, utilisation of codons may be markedly different in a single species between genes which are expressed at high and low levels. This bias is different in viruses, plants, bacteria and mammalian cells, and some species show a stronger bias away from a random codon selection than others. For example, humans and other mammals are less strongly biased than certain bacteria or viruses. For these reasons, there is a significant probability that a mammalian gene expressed in E. coli or a viral gene expressed in mammalian cells will have an inappropriate distribution of codons for efficient expression. It is believed that the presence in a heterologous DNA sequence of clusters of codons which are rarely observed in the host in which expression is to occur, is predictive of low heterologous expression levels in that host.

In consequence, codons preferred by a particular prokaryotic (for example E. coli or yeast) or eukaryotic host can be modified so as to encode the same MUC1 protein, but to differ from a wild type sequence. The process of codon modification may include any sequence, generated either manually or by computer software, where some or all of the codons of the native sequence of MUC1 are modified. Several method have been published (Nakamura et. al., Nucleic Acids Research 1996, 24:214-215; WO98/34640). One method is Syngene method, a modification of Calcgene method (R. S. Hale and G Thompson (Protein Expression and Purification Vol. 12 pp. 185-188 (1998)).

This process of codon modification of MUC1 may have some or all of the following benefits: 1) to improve expression of the gene product by replacing rare or infrequently used codons with more frequently used codons, 2) to remove or include restriction enzyme sites to facilitate downstream cloning and 3) to reduce the potential for homologous recombination between the insert sequence in the DNA vector and genomic sequences and 4) to improve the immune response in humans. The sequences of MUC1 advantageously have reduced recombination potential, but express to at least the same level as the wild type sequences. Due to the nature of the algorithms used by the SynGene programme to generate a codon modified sequence, it is possible to generate an extremely large number of different codon modified sequences which will perform a similar function. In brief, the codons are assigned using a statistical method to give synthetic gene having a codon frequency closer to that found naturally in highly expressed human genes such as β-Actin.

In an embodiment of the polynucleotides encoding immunogen for use in the present invention, where the immunogen is MUC-1, the codon usage pattern is altered from that typical of MUC-1 to more closely represent the codon bias of the target highly expressed human gene. The “codon usage coefficient” is a measure of how closely the codon pattern of a given polynucleotide sequence resembles that of a target species. Codon frequencies can be derived from literature sources for the highly expressed genes of many species (see e.g. Nakamura et al. Nucleic Acids Research 1996, 24:214-215). The codon frequencies for each of the 61 codons (expressed as the number of occurrences occurrence per 1000 codons of the selected class of genes) are normalised for each of the twenty natural amino acids, so that the value for the most frequently used codon for each amino acid is set to 1 and the frequencies for the less common codons are scaled to lie between zero and 1. Thus each of the 61 codons is assigned a value of 1 or lower for the highly expressed genes of the target species. In order to calculate a codon usage coefficient for a specific polynucleotide, relative to the highly expressed genes of that species, the scaled value for each codon of the specific polynucleotide are noted and the geometric mean of all these values is taken (by dividing the sum of the natural logs of these values by the total number of codons and take the anti-log). The coefficient will have a value between zero and 1 and the higher the coefficient the more codons in the polynucleotide are frequently used codons. If a polynucleotide sequence has a codon usage coefficient of 1, all of the codons are “most frequent” codons for highly expressed genes of the target species.

In one example of an immunogen for use in the present invention, the codon usage pattern of the polynucleotide may exclude codons representing <10% of the codons used for a particular amino acid. A relative synonymous codon usage (RSCU) value is the observed number of codons divided by the number expected if all codons for that amino acid were used equally frequently. A polynucleotide of the present invention may exclude codons with an RSCU value of less than 0.2 in highly expressed genes of the target organism. A polynucleotide of the present invention will generally have a codon usage coefficient for highly expressed human genes of greater than 0.6, greater than 0.65, or greater than 0.7. Codon usage tables for human can also be found in Genbank.

In comparison, a highly expressed beta actin gene has a RSCU of 0.747.

The codon usage table for a homo sapiens is set out below:

Codon usage for human (highly expressed) genes Jan. 24, 1991
(human_high.cod)

| AmAcid | Codon | Number | /1000 | Fraction . . . |
|---|---|---|---|---|
| Gly | GGG | 905.00 | 18.76 | 0.24 |
| Gly | GGA | 525.00 | 10.88 | 0.14 |
| Gly | GGT | 441.00 | 9.14 | 0.12 |
| Gly | GGC | 1867.00 | 38.70 | 0.50 |
| Glu | GAG | 2420.00 | 50.16 | 0.75 |
| Glu | GAA | 792.00 | 16.42 | 0.25 |
| Asp | GAT | 592.00 | 12.27 | 0.25 |
| Asp | GAC | 1821.00 | 37.75 | 0.75 |
| Val | GTG | 1866.00 | 38.68 | 0.64 |
| Val | GTA | 134.00 | 2.78 | 0.05 |
| Val | GTT | 198.00 | 4.10 | 0.07 |
| Val | GTC | 728.00 | 15.09 | 0.25 |
| Ala | GCG | 652.00 | 13.51 | 0.17 |
| Ala | GCA | 488.00 | 10.12 | 0.13 |
| Ala | GCT | 654.00 | 13.56 | 0.17 |
| Ala | GCC | 2057.00 | 42.64 | 0.53 |
| Arg | AGG | 512.00 | 10.61 | 0.18 |
| Arg | AGA | 298.00 | 6.18 | 0.10 |
| Ser | AGT | 354.00 | 7.34 | 0.10 |
| Ser | AGC | 1171.00 | 24.27 | 0.34 |
| Lys | AAG | 2117.00 | 43.88 | 0.82 |
| Lys | AAA | 471.00 | 9.76 | 0.18 |
| Asn | AAT | 314.00 | 6.51 | 0.22 |
| Asn | AAC | 1120.00 | 23.22 | 0.78 |
| Met | ATG | 1077.00 | 22.32 | 1.00 |
| Ile | ATA | 88.00 | 1.82 | 0.05 |
| Ile | ATT | 315.00 | 6.53 | 0.18 |
| Ile | ATC | 1369.00 | 28.38 | 0.77 |
| Thr | ACG | 405.00 | 8.40 | 0.15 |
| Thr | ACA | 373.00 | 7.73 | 0.14 |
| Thr | ACT | 358.00 | 7.42 | 0.14 |
| Thr | ACC | 1502.00 | 31.13 | 0.57 |
| Trp | TGG | 652.00 | 13.51 | 1.00 |
| End | TGA | 109.00 | 2.26 | 0.55 |
| Cys | TGT | 325.00 | 6.74 | 0.32 |
| Cys | TGC | 706.00 | 14.63 | 0.68 |
| End | TAG | 42.00 | 0.87 | 0.21 |
| End | TAA | 46.00 | 0.95 | 0.23 |
| Tyr | TAT | 360.00 | 7.46 | 0.26 |
| Tyr | TAC | 1042.00 | 21.60 | 0.74 |
| Leu | TTG | 313.00 | 6.49 | 0.06 |
| Leu | TTA | 76.00 | 1.58 | 0.02 |
| Phe | TTT | 336.00 | 6.96 | 0.20 |
| Phe | TTC | 1377.00 | 28.54 | 0.80 |
| Ser | TCG | 325.00 | 6.74 | 0.09 |
| Ser | TCA | 165.00 | 3.42 | 0.05 |
| Ser | TCT | 450.00 | 9.33 | 0.13 |
| Ser | TCC | 958.00 | 19.86 | 0.28 |
| Arg | CGG | 611.00 | 12.67 | 0.21 |
| Arg | CGA | 183.00 | 3.79 | 0.06 |
| Arg | CGT | 210.00 | 4.35 | 0.07 |
| Arg | CGC | 1086.00 | 22.51 | 0.37 |
| Gln | CAG | 2020.00 | 41.87 | 0.88 |
| Gln | CAA | 283.00 | 5.87 | 0.12 |
| His | CAT | 234.00 | 4.85 | 0.21 |
| His | CAC | 870.00 | 18.03 | 0.79 |
| Leu | CTG | 2884.00 | 59.78 | 0.58 |
| Leu | CTA | 166.00 | 3.44 | 0.03 |
| Leu | CTT | 238.00 | 4.93 | 0.05 |
| Leu | CTC | 1276.00 | 26.45 | 0.26 |
| Pro | CCG | 482.00 | 9.99 | 0.17 |
| Pro | CCA | 456.00 | 9.45 | 0.16 |
| Pro | CCT | 568.00 | 11.77 | 0.19 |
| Pro | CCC | 1410.00 | 29.23 | 0.48 |

Accordingly in one embodiment of the present invention where the nucleotide molecule encoding the immunogen component encode a MUC-1 immunogen, the nucleotide sequences are modified to more closely resemble the usage of a highly expressed human gene, such as β actin.

Any non-VNTR units of a MUC-1 immunogen component which may be used may be codon modified. The VNTR units when present may or may not be modified. In one embodiment, the codon-modified sequence is less than 80% identical to the corresponding non-VNTR unit of Muc-1.

When comparing polynucleotide sequences, two sequences are said to be “identical” if the sequence of nucleotides in the two sequences is the same when aligned for maximum correspondence, as described below.

Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Thus for an immunogen for use in the present invention, the non-repeat region of the codon-modified and the non-repeat region of optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

Examples of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides of the invention. Software for performing BLAST analyses is publicly available through the National Center for Biotechnology Information.

Such constructs are capable of raising both a cellular and also an antibody response that recognise MUC-1 expressing tumour cells. Inclusion of an adjuvant composition according to the present invention may improve the kinetics and functionality of the immune response to MUC.1.

The constructs can also contain altered repeat (VNTR units) such as reduced glycosylation mutants as described in WO01/46228.

Further MUC-1 constructs which may be used include the following, as described in WO03/100060, together with variants described therein:

1) 7 VNTR MUC-1 (ie Full Muc-1 with only 7 perfect repeats)
2) 7 VNTR MUC-1 Δss (As I, but also devoid of signal sequence)
3) 7 VNTR MUC-1 ΔTM ΔCYT (As 1, but devoid of Transmembrane and cytoplasmic domains)
4) 7 VNTR MUC-1 Δss ATM ΔCYT (As 3, but also devoid of signal sequence)
5) Truncated—MUC-1 (ie Full MUC-1 with no perfect repeats)
6) Truncated—MUC-1 Δss (As 5, but also devoid of signal sequence)
7) Truncated—MUC-1 ΔTM ΔCYT (As 5, but devoid of Transmembrane and cytoplasmic domains)
8) Truncated—MUC-1 Δss ATM ΔCYT (As 7, but also devoid of signal sequence)

In one embodiment, one or more of the imperfect VNTR units is mutated to reduce the potential for glycosylation, by altering a glycosylation site. The mutation may be a replacement, or can be an insertion or a deletion. Typically at least one threonine or serine is substituted with valine, isoleucine, alanine, asparagine, phenylalanine or tryptophan.

In a further embodiment, the gutted MUC-1 nucleic acid is provided with a restriction site at the junction of the leader sequence and the extracellular domain. Typically this restriction site is a Nhe1 site.

Her 2 neu antigens are disclosed inter alia, in U.S. Pat. No. 5,801,005. The Her 2 neu may comprise the entire extracellular domain (comprising approximately amino acid 1-645) or fragments thereof and at least an immunogenic portion of or the entire intracellular domain approximately the C terminal 580 amino acids. In particular, the intracellular portion should comprise the phosphorylation domain or fragments thereof. Such constructs are disclosed in WO00/44899. One construct is known as ECD PD, a second is known as ECD ΔPD. (See WO/00/44899.)

The her 2 neu as used herein can be derived from rat, mouse or human.

The vaccine may also contain antigens associated with tumour-support mechanisms (e.g. angiogenesis, tumour invasion) for example tie 2, VEGF.

Vaccines of the present invention may also be used for the prophylaxis or therapy of chronic disorders in addition to allergy, cancer or infectious diseases. Such chronic disorders are diseases such as asthma, atherosclerosis, and Alzheimer's and other auto-immune disorders. Vaccines for use as a contraceptive may also be considered.

Antigens relevant for the prophylaxis and the therapy of patients susceptible to or suffering from Alzheimer neurodegenerative disease are, in particular, the N terminal 39-43 amino acid fragment (AB the amyloid precursor protein and smaller fragments. This antigen is disclosed in the International Patent Application No. WO 99/27944—(Athena Neurosciences).

Potential self-antigens that could be included as vaccines for auto-immune disorders or as a contraceptive vaccine include: cytokines, hormones, growth factors or extracellular proteins, or a 4-helical cytokine, for example IL13. Cytokines include, for example, IL1, IL2, IL3, IL4, IL5, IL6, IL7, IL8, IL9, IL10, IL11, IL12, IL13, IL14, IL15, IL16, IL17, IL18, IL20, IL21, TNF, TGF, GMCSF, MCSF and OSM. 4-helical cytokines include IL2, IL3, IL4, IL5, IL13, GMCSF and MCSF. Hormones include, for example, luteinising hormone (LH), follicle stimulating hormone (FSH), chorionic gonadotropin (CG), VGF, GHrelin, agouti, agouti related protein and neuropeptide Y. Growth factors include, for example, VEGF.

The vaccines of the present invention are particularly suited for the immunotherapeutic treatment of diseases, such as chronic conditions and cancers, but also for the therapy of persistent infections. Accordingly the vaccines of the present invention are particularly suitable for the immunotherapy of infectious diseases, such as Tuberculosis (TB), HIV infections such as AIDS and Hepatitis B (HepB) virus infections.

In one embodiment the nucleic acid encodes one or more of the following antigens:—

HBV—PreS1 PreS2 and Surface env proteins, core and pol

### HCV—E1, E2, NS2, NS3, NS4A, NS4B, NS5A and B

HIV—gp120 gp40, gp160, p24, gag, pol, env, vif, vpr, vpu, tat, rev, nef

### Papilloma—E1, E2, E3, E4, E5, E6, E7, E8, L1, L2

### HSV—gL, gH, gM, gB, gC, gK, gE, gD, ICP47, ICP36, ICP4

Influenza—haemaggluttin, nucleoprotein
TB—Mycobacterial super oxide dismutase, 85A, 85B, MPT44, MPT59, MPT45, HSP10, HSP65, HSP70, HSP90, PPD 19 kDa Ag, PPD 38 kDa Ag.

It is envisaged that the present invention will be particularly effective at breaking tolerence against self-antigens, for example the cancer antigens P501S, or MUC-1. Such self-antigens may be used in the present invention.

In a further embodiment of the present invention, immunogen constructs of the present invention include a nucleic acid sequence encoding at least one heterologous T-cell epitope. These T cell epitopes may be incorporated within or at either end of the immunogen. T cell epitopes may be T helper epitopes. T cell epitopes include PADRE®, T-cell epitopes derived from bacterial proteins and toxins, such as Tetanus and Diphtheria toxins. For example, the P2 and P30 epitopes from Tetanus toxin may be used. Such epitopes may be part of a longer sequence. The epitopes may be incorporated within the nucleic acid molecules or at the 3′ or 5′ end of the sequence according to the invention.

Other fusion partners may be contemplated such as those derived from Hepatitis B core antigen, or tuberculosis. In an embodiment, a fusion partner derived from Mycobacterium tuberculosis, RA12, a sub-sequence (amino acids 192 to 323) of MTB32A (Skeiky et al Infection and Immunity (1999) 67: 3998-4007).

In an embodiment of the present invention, the immunogen is any one of the MUC-1 constructs as defined herein, fused to the promiscuous T cell epitope PADRE.

Yet other immunological fusion partners, include for example, protein D from Haemophilus influenza B (WO91/18926) or a portion (typically the C-terminal portion) of LytA derived from Streptococcus pneumoniae (CLytA; Biotechnology 10: 795-798, 1992), which may be fused to another partner such as P2 ie. ClytA-P2-CLytA (CPC), as described in WO03/104272. WO99/40188 describes inter alia fusion proteins comprising MAGE antigens with a His tails and a C-LytA portion at the N-terminus of the molecule; nucleic acid sequences encoding such fusion proteins may comprise component (iii) of the present invention.

Further immunogen constructs which may be encoded by a nucleotide comprising component (iii) of the present invention may therefore include:
Immunogen—C-LytA repeats1-4-P2 epitope (inserted in or replacing C-LytA repeat5)-C-LytA repeat6
C-LytA repeats1-4-P2 epitope (inserted in or replacing C-LytA repeat5)-C-LytA repeats immunogen
Immunogen—C-LytA repeat2-5-P2 epitope (inserted into C-LytA repeat6)
C-LytA2-5-P2 epitope (inserted into C-LytA repeat6) immunogen.
Immunogen C-LytA repeats1-5-P2 epitope-inserted in C-LytA repeat6
C-LytA repeats1-5-P2 epitope-inserted in C-LytA repeat6-immunogen
Immunogen—P2 epitope inserted into C-LytA repeat1-C-LytA repeats2-5
P2 epitope inserted into C-LytA repeat1-C-LytA repeats2-5-immunogen
Immunogen—P2 epitope inserted into C-LytA repeat1-C-LytA repeats2-6
P2 epitope inserted into C-LytA repeat1-C-LytA repeats2-6-immunogen
Immunogen—C-LytA repeat1-P2 epitope inserted into C-LytA repeat2-C-LytA repeats3-6
C-LytA repeat1-P2 epitope inserted into C-LytA repeat2-C-LytA repeats3-6-immunogen;
   where “inserted into” means at any place into the repeat for example between residue 1 and 2, or between 2 and 3, etc.

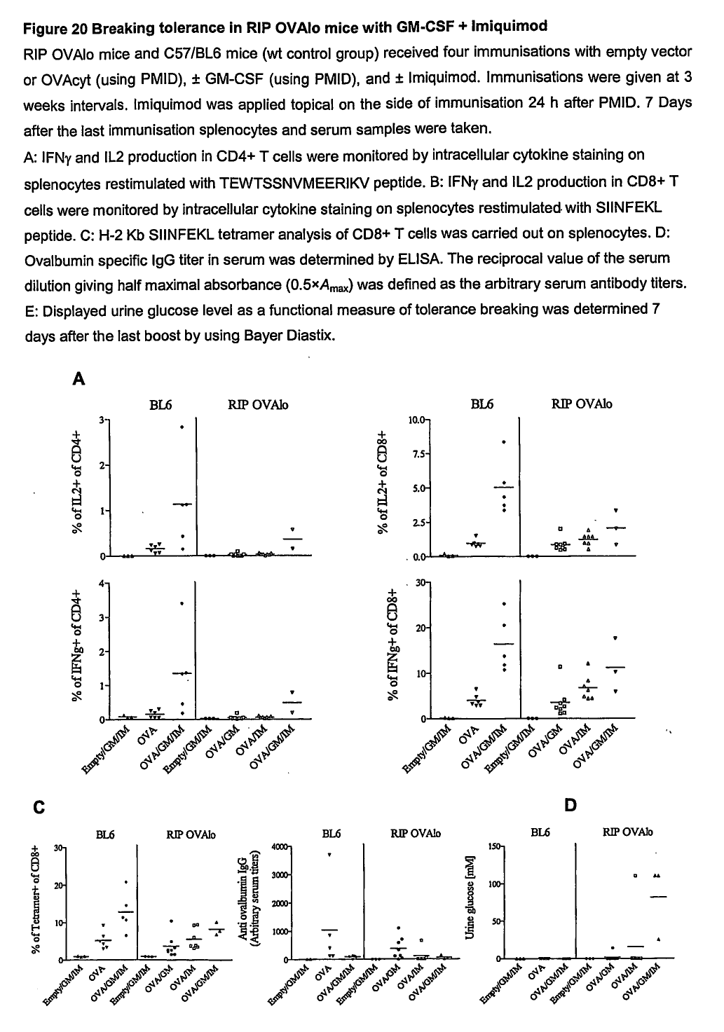

The promiscuous T helper epitope may be inserted within a repeat region for example C-LytA repeats 2-5_-C-LytA repeat 6a-P2 epitope-C-LytA repeat 6b, where the P2 epitope is inserted within the sixth repeat (see FIG. 20 of WO03/104272). In other embodiments the C-terminal end of CPL1 (C-CPL1) may be used as an alternative to C-LytA.

Alternatively, the P2 epitope in the above constructs may be replaced by other promiscuous T epitopes, for example P30.

Particularly illustrative immunogens comprise a sequence of at least 10 contiguous amino acids, 20, 30, 40, 50, 60, 70, 80, 90, 100, 110, 120, 130, 140, 150, 160, 170, 180 amino acids of a tumour associated or tissue specific protein fused to the fusion partner.

According to a further aspect of the invention, expression vectors are provided which comprise and are capable of directing the expression of each polynucleotide sequence of the invention. The vector may be suitable for driving expression of heterologous DNA in bacterial insect or mammalian cells, particularly human cells.

Also provided are the use of a vaccine or immunogenic composition according to the invention, or of a vector according to the invention, in the treatment or prophylaxis of MUC-1 or P501S expressing tumour or metastases.

The present invention also provides methods of treating or preventing MUC-1 or P501S expressing tumour, any symptoms or diseases associated therewith including metastases, comprising administering an effective amount of the vaccine or immunogenic composition according to the invention.

The present invention is not limited to vaccines comprising nucleic acid encoding MUC-1.

The nucleotide sequence may be RNA or DNA including genomic DNA, synthetic DNA or cDNA. In one embodiment the nucleotide sequence is a DNA sequence, or a cDNA sequence. In order to obtain expression of the antigenic peptide within mammalian cells, it is necessary for the nucleotide sequence encoding the antigenic peptide to be presented in an appropriate vector system. By “appropriate vector” as used herein is meant any vector that will enable the antigenic peptide to be expressed within a mammal in sufficient quantities to evoke an immune response.

For example, the vector selected may comprise a plasmid, promoter and polyadenylation/transcriptional termination sequence arranged in the correct order to obtain expression of the antigenic peptides. The construction of vectors which include these components and optionally other components such as enhancers, restriction enzyme sites and selection genes, such as antibiotic resistance genes, is well known to persons skilled in the art and is explained in detail in Maniatis et al “Molecular Cloning: A Laboratory Manual”, Cold Spring Harbour Laboratory, Cold Spring Harbour Press, Vols 1-3, 2^{nd} Edition, 1989.

To prevent the plasmids replicating within the mammalian host and integrating within the chromosomal DNA of the animal, the plasmid may be produced without an origin of replication that is functional in eukaryotic cells.

The methods and compositions according to the present invention can be used in relation to prophylactic or treatment procedures of all mammals including, for example, domestic animals, laboratory animals, farm animals, captive wild animals or, in one embodiment, humans.

As discussed above, the present invention includes the use of expression vectors that encode the adjuvant components (i) and/or (ii), or antigen or immunogen components (iii) of the invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al. Molecular Cloning: a Laboratory Manual. 2^{nd} Edition. CSH Laboratory Press. (1989).

A polynucleotide, or for use in the invention in a vector, may be operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be, for example, plasmids, artificial chromosomes (e.g. BAC, PAC, YAC), virus or phage vectors provided with an origin of replication, optionally a promoter for the expression of the polynucleotide and optionally a regulator of the promoter. The vectors may contain one or more selectable marker genes, for example an ampicillin or kanamycin resistance gene in the case of a bacterial plasmid or a resistance gene for a fungal vector. Vectors may be used in vitro, for example for the production of DNA or RNA or used to transfect or transform a host cell, for example, a mammalian host cell e.g. for the production of protein encoded by the vector. The vectors may also be adapted to be used in vivo, for example in a method of DNA vaccination or of gene therapy.

Promoters and other expression regulation signals may be selected to be compatible with the host cell for which expression is designed. For example, mammalian promoters include the metallothionein promoter, which can be induced in response to heavy metals such as cadmium, and the β-actin promoter. Viral promoters such as the SV40 large T antigen promoter, human cytomegalovirus (CMV) immediate early (1E) promoter, rous sarcoma virus LTR promoter, adenovirus promoter, or a HPV promoter, particularly the HPV upstream regulatory region (URR) may also be used. All these promoters are well described and readily available in the art.

One promoter element is the CMV immediate early promoter devoid of intron A, but including exon 1 (WO02/36792). Accordingly there is provided a vector comprising a polynucleotide of the invention under the control of HCMV IE early promoter.

Examples of suitable viral vectors include herpes simplex viral vectors, vaccinia or alpha-virus vectors and retroviruses, including lentiviruses, adenoviruses and adeno-associated viruses. Gene transfer techniques using these viruses are known to those skilled in the art. Retrovirus vectors for example may be used to stably integrate the polynucleotide of the invention into the host genome, although such recombination is not preferred. Replication-defective adenovirus vectors by contrast remain episomal and therefore allow transient expression. Vectors capable of driving expression in insect cells (for example baculovirus vectors), in human cells or in bacteria may be employed in order to produce quantities of the HIV protein encoded by the polynucleotides of the present invention, for example for use as subunit vaccines or in immunoassays. The polynucleotides of the invention have particular utility in viral vaccines as previous attempts to generate full-length vaccinia constructs have been unsuccessful.

In one embodiment of the present invention, viral vectors may be used which comprise an adenoviral nucleic acid sequence selected from C1, Pan 5, Pan 6, Pan 7 C68 (Pan 9), SV1, SV25 and SV 39, as described in published PCT application WO 03/046124, the entirety of which earlier publication is incorporated herein by reference.

Bacterial vectors, such as attenuated Salmonella or Listeria may alternatively be used. The polynucleotides according to the invention have utility in the production by expression of the encoded proteins, which expression may take place in vitro, in vivo or ex vivo. The nucleotides may therefore be involved in recombinant protein synthesis, for example to increase yields, or indeed may find use as therapeutic agents in their own right, utilised in DNA vaccination techniques. Where the polynucleotides of the present invention are used in the production of the encoded proteins in vitro or ex vivo, cells, for example in cell culture, will be modified to include the polynucleotide to be expressed. Such cells include transient, or stable mammalian cell lines. Particular examples of cells which may be modified by insertion of vectors encoding for a polypeptide according to the invention include mammalian HEK293T, CHO, HeLa, 293 and COS cells. The cell line selected may be one which is not only stable, but also allows for mature glycosylation and cell surface expression of a polypeptide. Expression may be achieved in transformed oocytes. A polypeptide may be expressed from a polynucleotide of the present invention, in cells of a transgenic non-human animal, such as a mouse. A transgenic non-human animal expressing a polypeptide from a polynucleotide of the invention is included within the scope of the invention.

The invention further provides a method of vaccinating a mammalian subject which comprises administering thereto an effective amount of such a vaccine or vaccine composition. Expression vectors for use in DNA vaccines, vaccine compositions and immunotherapeutics may be plasmid vectors.

The immunogen component comprising a vector which comprises the nucleotide sequence encoding an antigenic peptide can be administered in a variety of manners. It is possible for the vector to be administered in a naked form (that is as naked nucleotide sequence not in association with liposomal formulations, with viral vectors or transfection facilitating proteins) suspended in an appropriate medium, for example a buffered saline solution such as PBS and then injected intramuscularly, subcutaneously, intraperitonally or intravenously, although some earlier data suggests that intramuscular or subcutaneous injection may be used (Brohm et al Vaccine 16 No. 9/10 pp 949-954 (1998), the disclosure of which is included herein in its entirety by way of reference). It is additionally possible for the vectors to be encapsulated by, for example, liposomes or within polylactide co-glycolide (PLG) particles (25) for administration via the oral, nasal or pulmonary routes in addition to the routes detailed above.

It is also possible, according to one embodiment of the invention, for intradermal administration of the immunogen component, for example via use of gene-gun (particularly particle bombardment) administration techniques. Such techniques may involve coating of the immunogen component on to gold beads which are then administered under high pressure into the epidermis, such as, for example, as described in Haynes et al J. Biotechnology 44: 37-42 (1996).

In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide, are accelerated to high speed within a helium gas jet generated by a hand held device, propelling the particles into a target tissue of interest, typically the skin. The particles may be gold beads of a 0.4-4.0 μm, or 0.6-2.0 μm diameter and the DNA conjugate coated onto these and then encased in a cartridge or cassette for placing into the “gene gun”.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

The nucleic acid vaccine may also be delivered by means of micro needles, which may be coated with a composition of the invention or delivered via the micro-needle from a reservoir.

The vectors which comprise the nucleotide sequences encoding antigenic peptides are administered in such amount as will be prophylactically or therapeutically effective. The quantity to be administered, is generally in the range of one picogram to 1 milligram, or 1 picogram to 10 micrograms for particle-mediated delivery, and 10 micrograms to 1 milligram for other routes of nucleotide per dose. The exact quantity may vary considerably depending on the species and weight of the mammal being immunised, the route of administration, the potency and dose of the adjuvant components, the nature of the disease state being treated or protected against, the capacity of the subject's immune system to produce an immune response and the degree of protection or therapeutic efficacy desired. Based upon these variables, a medical or veterinary practitioner will readily be able to determine the appropriate dosage level.

It is possible for the immunogen component (iii) comprising the nucleotide sequence encoding the antigenic peptide, and the adjuvant components (i) and (ii) to be administered on a once off basis or to be administered repeatedly, for example, between 1 and 7 times, or between 1 and 4 times, at intervals between about 4 weeks and about 18 months. Once again, however, this treatment regime will be significantly varied depending upon the size of the patient, the disease which is being treated/protected against, the amount of nucleotide sequence administered, the route of administration, and other factors which would be apparent to a skilled medical practitioner. The patient may receive one or more other anti cancer drugs as part of their overall treatment regime.

Once again, depending upon the type of variables listed above, the dose of administration of the TLR agonist will also vary, but may, for example, range between about 0.1 mg per kg to about 100 mg per kg, where “per kg” refers to the body weight of the mammal concerned. This administration of the TLR agonist amine derivative may be repeated with each subsequent or booster administration of the nucleotide sequence. The administration dose may be between about 0.5 mg per kg to about 5 mg per kg, or about 1 mg/kg or 1 mg/kg. Where the TLR agonist is resiquimod or imiquimod, the dose may be 1 mg/kg. Where the TLR agonist is imiquimod, Aldara™ cream (5% imiquimod; 3M) may be used, and applied topically at or near the site of administration. In one embodiment of the invention, one 12.5 mg packet (3M) of 5% Aldara™ cream may be used, alternatively more than one packet of Aldara™ cream may be used. In a further embodiment of the invention, a fraction of a packet may be used: for example at or about 20%, 25%, 33% or 50% of a packet may be used at or near each site.

While it is possible for the TLR agonist adjuvant component to comprise an imidazoquinoline molecule or derivative thereof to be administered in the raw chemical state, administration may be in the form of a pharmaceutical formulation. That is, the TLR agonist adjuvant component may comprise the imidazoquinoline molecule or derivative thereof combined with one or more pharmaceutically or veterinarily acceptable carriers, and optionally other therapeutic ingredients. The carrier(s) must be “acceptable” in the sense of being compatible with other ingredients within the formulation, and not deleterious to the recipient thereof. The nature of the formulations will naturally vary according to the intended administration route, and may be prepared by methods well known in the pharmaceutical art. All methods of preparing formulations include the step of bringing into association an imidazoquinoline molecule or derivative thereof with an appropriate carrier or carriers. Carriers include a cream formulation, or alternatively PBS or water. In general, the formulations are prepared by uniformly and intimately bringing into association the derivative with liquid carriers or finely divided solid carriers, or both, and then, if necessary, shaping the product into the desired formulation. Formulations of the present invention suitable for oral administration may be presented as discrete units such as capsules, cachets or tablets each containing a pre-determined amount of the active ingredient; as a powder or granules; as a solution or a suspension in an aqueous liquid or a non-aqueous liquid; or as an oil-in-water liquid emulsion or a water-in-oil emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent.

The tablets may optionally be coated or scored and may be formulated so as to provide slow or controlled release of the active ingredient.

Formulations for injection via, for example, the intramuscular, intraperitoneal, or subcutaneous administration routes include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampoules and vials, and may be stored in a freeze-dried (lyophilised) condition requiring only the addition of the sterile liquid carrier, for example, water for injections, immediately prior to use. Extemporaneous injection solutions and suspensions may be prepared from sterile powders, granules and tablets of the kind previously described. Formulations suitable for pulmonary administration via the buccal or nasal cavity are presented such that particles containing the active ingredient, desirably having a diameter in the range of 0.5 to 7 microns, are delivered into the bronchial tree of the recipient. Possibilities for such formulations are that they are in the form of finely comminuted powders which may conveniently be presented either in a piercable capsule, suitably of, for example, gelatine, for use in an inhalation device, or alternatively, as a self-propelling formulation comprising active ingredient, a suitable liquid propellant and optionally, other ingredients such as surfactant and/or a solid diluent. Self-propelling formulations may also be employed wherein the active ingredient is dispensed in the form of droplets of a solution or suspension. Such self-propelling formulations are analogous to those known in the art and may be prepared by established procedures. They are suitably provided with either a manually-operable or automatically functioning valve having the desired spray characteristics; advantageously the valve is of a metered type delivering a fixed volume, for example, 50 to 100 μL, upon each operation thereof.

In a further possibility, the adjuvant component may be in the form of a solution for use in an atomiser or nebuliser whereby an accelerated airstream or ultrasonic agitation is employed to produce a find droplet mist for inhalation.

Formulations suitable for intranasal administration generally include presentations similar to those described above for pulmonary administration, although such formulations may have a particle diameter in the range of about 10 to about 200 microns, to enable retention within the nasal cavity. This may be achieved by, as appropriate, use of a powder of a suitable particle size, or choice of an appropriate valve. Other suitable formulations include coarse powders having a particle diameter in the range of about 20 to about 500 microns, for administration by rapid inhalation through the nasal passage from a container held close up to the nose, and nasal drops comprising about 0.2 to 5% w/w of the active ingredient in aqueous or oily solutions. In one embodiment of the invention, it is possible for the vector which comprises the nucleotide sequence encoding the antigenic peptide to be administered within the same formulation as the 1H-imidazo[4,5-c]quinolin-4-amine derivative. Hence in this embodiment, the immunogenic and the adjuvant component are found within the same formulation.

In one embodiment adjuvant component (ii) and immunogen component (iii) are prepared in forms suitable for gene-gun administration, and are administered via that route concomitant to administration of the nucleotide sequence encoding immunogen. For preparation of formulations suitable for use in this manner, it may be necessary for the adjuvant component (ii) and immunogen component (iii) to be lyophilised and adhered onto, for example, gold beads which are suited for gene-gun administration. In this embodiment, adjuvant component (i) may be administered sequentially, in a separate composition.

In an alternative embodiment, adjuvant component (i), or (ii), or both, may be administered as a dry powder, via high pressure gas propulsion. At least one adjuvant component may be concomitant to administration of the nucleotide sequence encoding immunogen; adjuvant component (ii) may be administered concomitant to administration of the immunogen component.

Even if not formulated together, it may be appropriate for adjuvant components (i) and (ii) to be administered at or about the same administration site as the nucleotide sequence.

Other details of pharmaceutical preparations can be found in Remington's Pharmaceutical Sciences, Mack Publishing Company, Easton, Pa. (1985), the disclosure of which is included herein in its entirety, by way of reference.

The adjuvant components specified herein can similarly be administered via a variety of different administration routes, such as for example, via the oral, nasal, pulmonary, intramuscular, subcutaneous, intradermal or topical routes. The components may be administered via the intradermal, subcutaneous or topical routes.

Administration of the adjuvant may take place between about 14 days prior to and about 14 days post administration of the nucleotide sequence, or between about 1 day prior to and about 3 days post administration of the nucleotide sequence. Nucleotide sequence encoding GM-CSF may be administered concomitantly with the administration of the nucleotide sequence encoding immunogen, and the component which is a TLR agonist provided sequentially. The component which is a TLR agonist may be given about or exactly 7, 6, 5, 4, 3, 2, or 1 day(s) or about or exactly 24, 22, 20, 18, 16, 14, 12, 10, 9, 8, 7, 6, 5, 4, 3, 2, or one hour(s) before the antigen component. The component which is a TLR agonist may be given about or exactly 7, 6, 5, 4, 3, 2 or 1 day(s) or about or exactly 24, 22, 20, 18, 16, 14, 12, 10, 9, 8, 7, 6, 5, 4, 3, 2, or one hour(s) after the antigen component.

The component which is a TLR agonist may be given at or about 24 hours after the remaining components. An advantage of giving the TLR agonist component after administration of components (ii) and (iii) is that delivery of components (ii) and (iii) may lead to induction of IFNγ in the locality of delivery; this may lead to upregulation of TLRs, such as up-regulation of TLRs 7 and/or 8, leading to increased responsiveness to the TLR agonist.

In one embodiment of the present invention, components (ii) and (iii) are in a formulation suitable for simultaneous administration by gene gun delivery, and adjuvant component (i) is provided in a separate cream formulation, for sequential topical administration.

Suitable techniques for introducing the naked. polynucleotide or vector into a patient also include topical application with an appropriate vehicle. The nucleic acid may be administered topically to the skin, or to mucosal surfaces for example by intranasal, oral, intravaginal or intrarectal administration. The naked polynucleotide or vector may be present together with a pharmaceutically acceptable excipient, such as phosphate buffered saline (PBS). DNA uptake may be further facilitated by use of facilitating agents such as bupivacaine, either separately or included in the DNA formulation. Other methods of administering the nucleic acid directly to a recipient include ultrasound, electrical stimulation, electroporation and microseeding which is described in U.S. Pat. No. 5,697,901.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered.

A nucleic acid sequence of the present invention may also be administered by means of transformed cells. Such cells include cells harvested from a subject. The naked polynucleotide or vector of the present invention can be introduced into such cells in vitro and the transformed cells can later be returned to the subject. The polynucleotide of the invention may integrate into nucleic acid already present in a cell by homologous recombination events. A transformed cell may, if desired, be grown up in vitro and one or more of the resultant cells may be used in the present invention. Cells can be provided at an appropriate site in a patient by known surgical or microsurgical techniques (e.g. grafting, micro-injection, etc.)

The present inventors have demonstrated that the combination of TLR agonist with GM-CSF, when used as adjuvants in DNA vaccination, is capable of increasing cell-mediated immunology responses, in particular after a prime injection. The term adjuvant or adjuvant component as used herein is intended to convey that the derivatives or component comprising the derivatives act to enhance and/or alter the body's response to an immunogen in a desired fashion. So, for example, an adjuvant may be used to shift an immune response to a predominately Th1 response, or to increase both types of responses.

An inducer of a TH1 type of immune response enables a cell mediated response to be generated. High levels of Th1-type cytokines tend to favour the induction of cell mediated immune responses to the given antigen, whilst high levels of Th2-type cytokines tend to favour the induction of humoral immune responses to the antigen.

It is important to remember that the distinction of Th1 and Th2-type immune response is not absolute. In reality an individual will support an immune response which is described as being predominantly Th1 or predominantly Th2. However, it is often convenient to consider the families of cytokines in terms of that described in murine CD4+ve T cell clones by Mosmann and Coffman (Mosmann, T. R. and Coffman, R. L. (1989) TH1 and TH2 cells: different patterns of lymphokine secretion lead to different functional properties. Annual Review of Immunology, 7, p 145-173). Traditionally, Th1-type responses are associated with the production of the IFN-γ and IL-2 cytokines by T-lymphocytes. Other cytokines often directly associated with the induction of Th1-type immune responses are not produced by T-cells, such as IL-12. In contrast, Th2-type responses are associated with the secretion of 11-4, IL-5, IL-6, IL-10.

The invention will now be described further, with reference to the following non-limiting examples:

### EXAMPLES

### Introduction

The experiments demonstrate the use of a nucleotide molecule encoding GM-CSF and a TLR agonist to enhance the cellular immune response to an antigenic peptide. Significant differences in the immunogenicity have been observed; use of an adjuvant comprising a nucleotide encoding GM-CSF, together with a TLR agonist may improve the kinetics and functionality of an immune response to an antigen, as can be seen from the following experiments and which can be further demonstrated by following protocols outlined herein and protocols well known in the art.

### Materials & Methods

### Materials & Methods

### I Construction of Expression Vectors: OVAcyt, 7VNTRMuc1, HIV RNG and GM-CSF Plasmid

### Construction of OVAcyt Plasmid

A gene encoding a non-secreted form of chicken ovalbumin was constructed by deleting the secretion signal (a.a. 20-145) of the wild type chicken ova gene. This truncated gene is termed OVAcyt to signify that it is a non-secreted, cytoplasmic form of the ovalbumin protein. This gene was amplified by PCR using primers incorporating restriction sites to enable ligation into the DNA vaccine vector p7313 (details included in WO 02/08435, the entirety of which earlier publication is incorporated herein by reference).

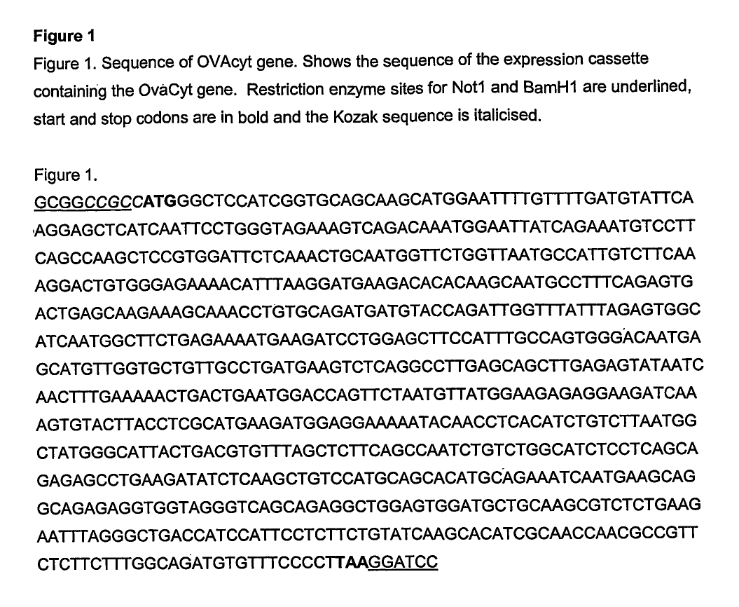

FIG. 1 shows the sequence of the expression cassette containing the OvaCyt gene. Restriction enzyme sites for Not1 and BamH1 are underlined, start and stop codons are in bold and the Kozak sequence is italicised.

### Construction of GMCSF Plasmid

Mouse GM-CSF was cloned from a cDNA library and cloned into the expression vector pVACss2. This cDNA clone was used as a template to amplify the mGM-CSF open reading frame by PCR, using primers incorporating a Kozac sequence, start codon and restriction enzyme sites to enable cloning into the DNA vaccine vector p7313 (WO 02/08435 as above). FIG. 2 shows the coding sequence for this mGM-CSF expression cassette.

In FIG. 2, restriction enzyme sites for Nhe1 and Asc1 are shown underlined, the start and stop codons are in bold and the Kozak sequence is in italics.

### Construction of RNG Plasmid

The inactivated codon optimised RT, truncated Nef and p17/p24 portion of the codon optimised gag gene from the HIV-1 clade B strain HXB2 downstream of an Iowa length HCMV promoter+exon1, and upstream of a rabbit β-globin poly-adenylation signal.

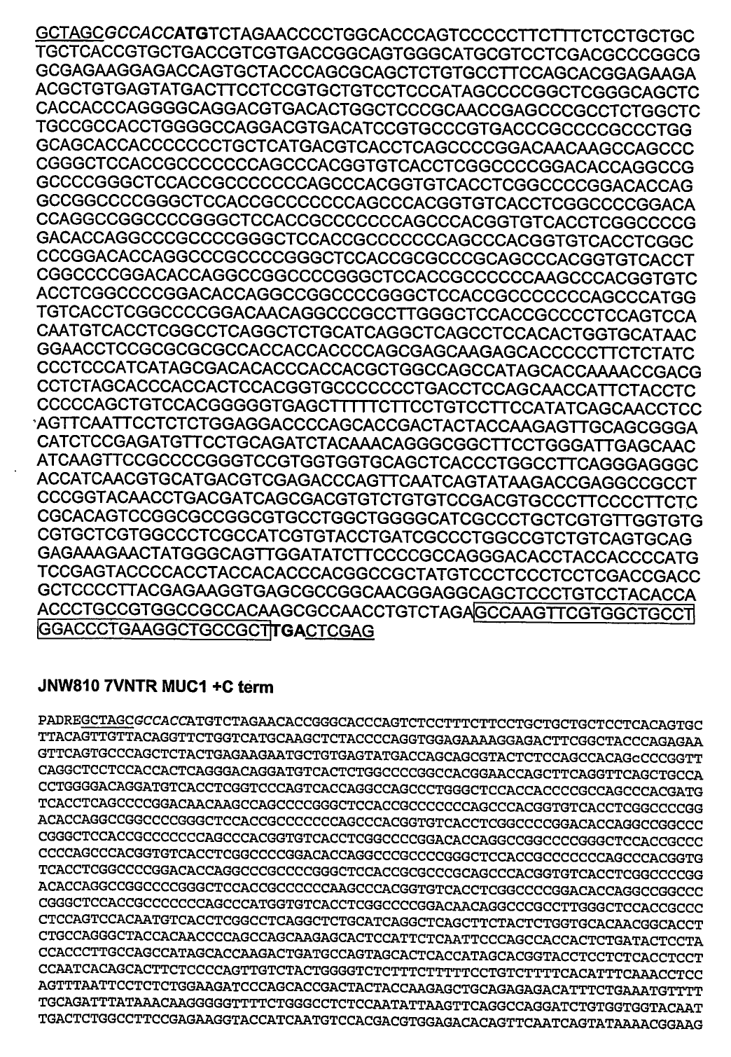

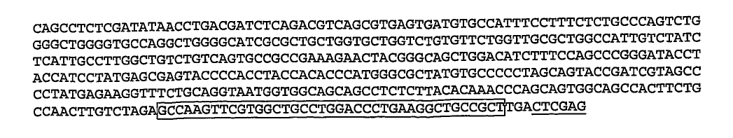

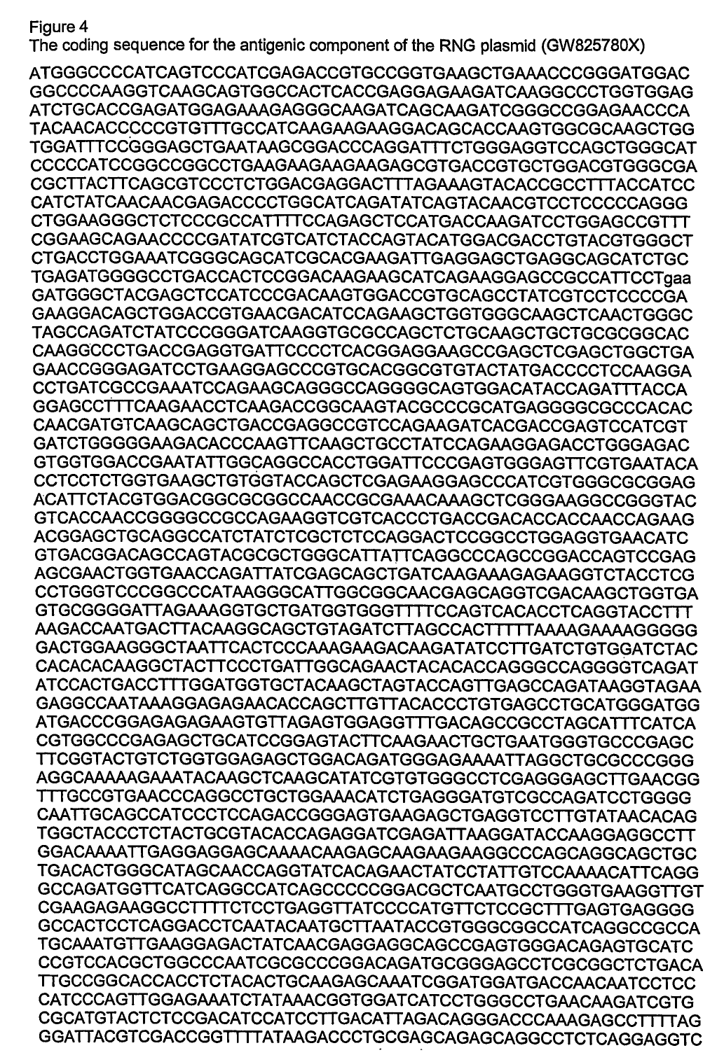

The order of the genes within the construct was achieved by PCR amplification of the RT-trNef and p17p24 genes from p731-Tgm. PCR stitching of the two DNA fragments was performed and the 3 kb product gel purified and NotI/BamHI cut prior to ligation with NotI/BamHI digested p7313ie. The sequence is shown in FIG. 4.

### Generation of MUC-1 Constructs

### Construction of a MUC1 Expression Vector Containing Seven VNTR Units

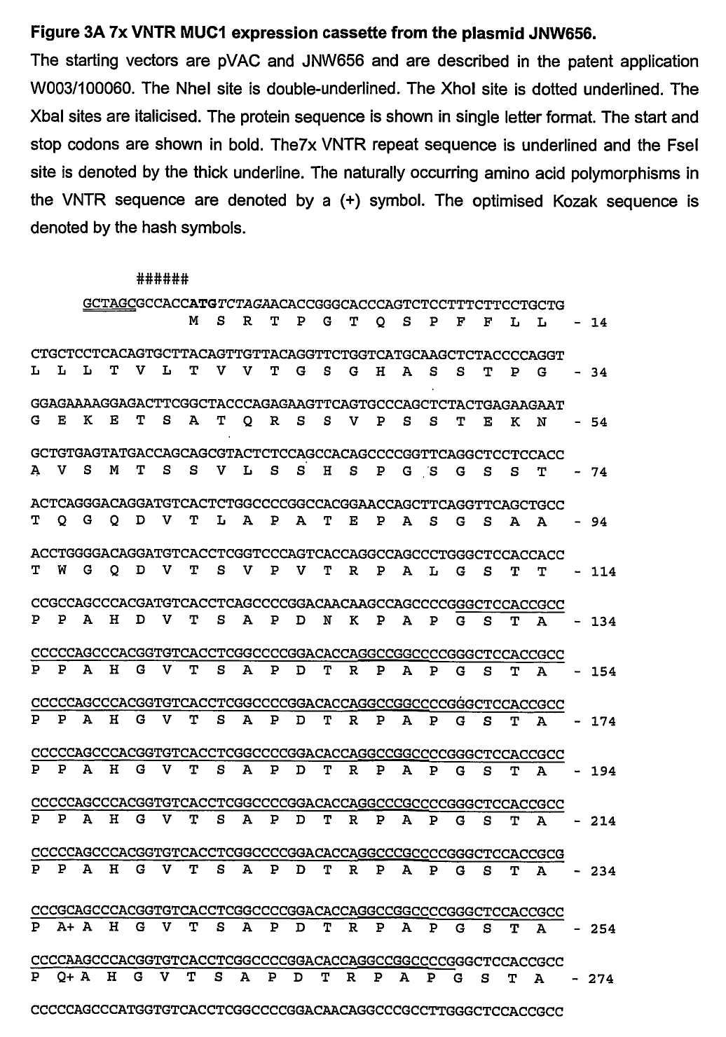

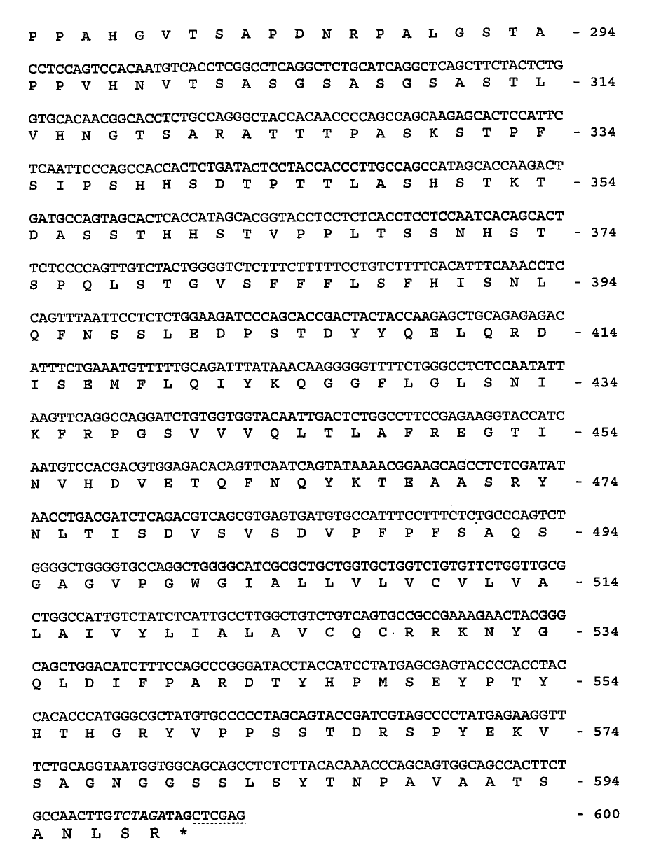

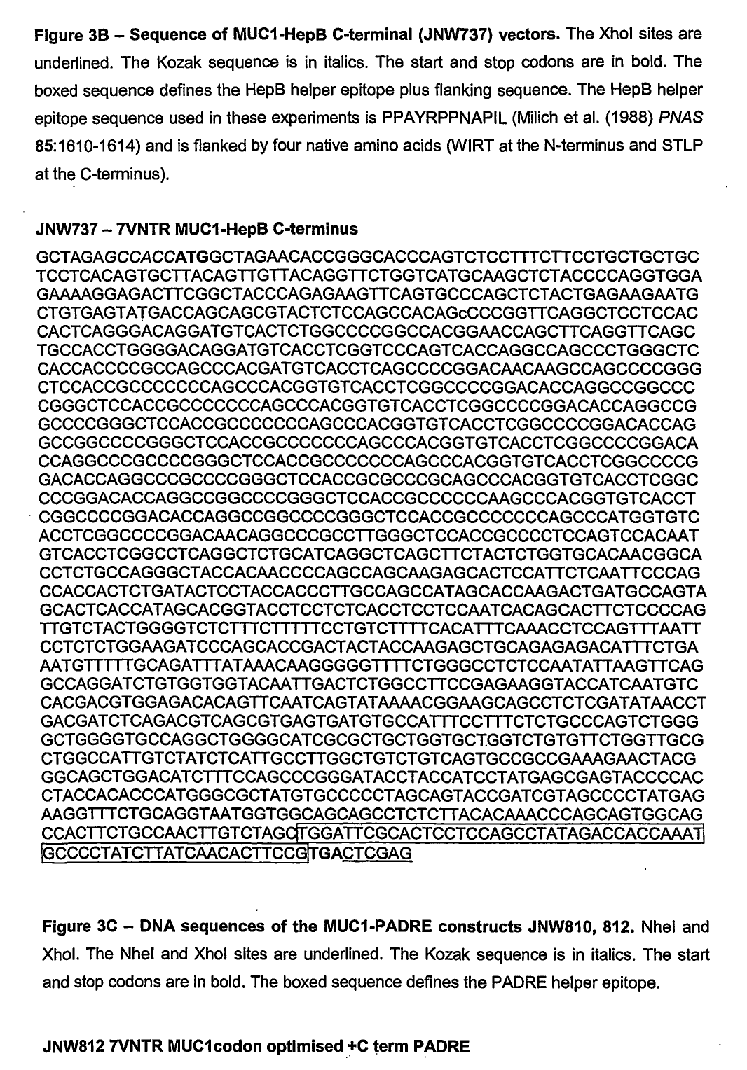

The construction of this vector is detailed in patent application WO03/100060, the disclosure of which is incorporated herein by reference, and its sequence is shown in FIG. 3A.

Construction of a MUC1 Expression Cassette with a HepB Helper Epitope Inserted at the C-Terminus of MUC1

A two-step process was used to insert the HepB helper epitope at the C-terminus of MUC1. A short DNA linker encoding the epitope was generated by annealing two oligos, FORA and REVA. FOR primer 10 pmol, REV primer 10 pmol, 1×T4 DNA ligase buffer and 10U T4 polynucleotide kinase was mixed in a total volume 20 μl, incubated for 2 hrs at 37° C. and annealed by heating first to 95° C. for 2 minutes and then cooling at a rate off −0.1° C./s. Hold at 4° C. The resulting linkers were ligated into the NheI/XhoI site of pVAC, generating vectors JNW729 (C-terminal). The MUC1 expression cassette was excised from vector JNW656 on an XbaI cassette and cloned into the NheI sites of vectors JNW729, generating vectors JNW737 (C-terminal). All vectors were sequence verified. The sequence of JNW737 is shown in FIG. 3B, with the helper epitope sequence boxed.

Construction of a MUC1 Expression Cassette with a PADRE Helper Epitope Inserted at the C-Terminus of MUC1

A C-terminal fusion was generated by first inserting a short linker into pVAC1. The linker was created by annealing the two primers PADREFOR and PADREREV and cloning the linker into pVAC1 via the NheI and XhoI sites, generating vector JNW800. Into JNW800, the 7×VNTR MUC1 expression cassette from JNW656 (7×VNTR MUC1) and JNW758 (codon optimised 7×VNTR MUC1, see patent application VB60033) was inserted by excising the MUC1 cassette on an XbaI fragment and cloning into the XbaI site, generating the following two vectors

### 7×VNTR MUC1 C-term PADRE: JNW810

7×VNTR MUC1 (codon optimised) C-term PADRE: JNW812

The sequencing of the MUC1 expression cassette and PADRE epitope from JNW810 and JNW812 are shown in FIG. 3C.

### 2 Testing of Constructs—Materials

### Animals

CBAB6.F1 is a cross of C57BI6 mice and CBA mice and they are the wild type background for the MUC1 Tg mice used. MUC1 Tg mice were obtained from the Imperial Cancer Research Fund and they express human MUC1 under the control of the human MUC1 promoter (Peat et al, 1992). MUC1 expression pattern on those mice is very similar to the profile of expression seen in human tissues. C57/bl6 or Balb/C obtained from Charles River were used for studies involving p7313OVAcyt and p7313RNG. RIP-OVAlo mice were bred in house at GSK.

2.1 Co Delivery of Two Plasmids: p7313 OVAcyt (Plasmid Encoding Antigen) p7313RNG (Plasmid Encoding Antigen, or pVAC 7VNTR Muc1 (Plasmid Encoding Antigen) and p7313 GMCSF Plasmids (Plasmid Encoding GM-CSF)

Plasmid DNA was precipitated onto 2 μm diameter gold beads using calcium chloride and spermidine. Equal amounts of plasmids encoding antigen (p7313OVAcyt, p7313RNG, pVAC7VNTRMuc1, pVAC7VNTRMuc1-PADRE or pVAC7VNTRMuc1-HepB) and p7313GMCSF plasmids were mixed and co-precipitated so that all beads were coated with a mixture of the 2 plasmids ensuring delivery of both plasmids to the same cell. Unless otherwise stated both the antigen and GMCSF were loaded at 0.5 ug/cartridge. Where lower doses of antigen were used the GMCSF loading remained at 0.5 ug and the total DNA on the cartridge was adjusted to 1 ug using p7313empty or pVACempty plasmids. Loaded beads were coated onto Tefzel tubing as described in, for example, Eisenbraum, et al. 1993. DNA Cell Biol. 12:791-797; Pertmer et al, 1996 J. Virol. 70:6119-6125). Particle bombardment was performed using the Accell gene delivery (PCT WO 95/19799; incorporated herein by reference). Female C56BI/6 mice were immunised with 2 administrations of plasmid at each time point as detailed in the results section, one on each side of the abdomen after shaving. The total dose of DNA at each time point was 2 μg. Where imiquimod was delivered this was applied topically in a cream formulation over the immunisation site, 24 hours following immunisation. 20 μl of 5% Aldara™ cream (3M) was applied at each immunisation site. In the case of minipigs 4 immunisations of 1 ug each were given on the abdomen (after shaving).

### Co-Coating of CpG Oligonucleotides

The CpG oligonucleotides were co-coated onto gold beads using the same methodology as co-coating of plasmids. The oligos were mixed with the DNA at a ratio of 10:1 oligo:plasmid. We have shown that plasmid is not displaced by the oligonucleotides and estimate that 10% of the oligonucleotide is precipitated onto the beads resulting in a 1:1 ratio on the cartridges. Co-coating with a 10:1 ratio of oligo to plasmid results in higher incorporation of oligo on the cartridges compared with a 1:1 ratio. The ODNs used in this study are listed in Table 1. The PTO ODNs CpG1826 (stimulatory CpG) and GpC1745 (non stimulatory oligo) and DNA ODNs were synthesised by MWG-Biotech AG.

TABLE 1
List of oligonucleotides used in this study.

| | Descrip- | |
|---|---|---|
| Oligonucleotide | tion | Sequence |
| CpG1826 | 20mer | 5′-tccatga [symbol] ttcctga [symbol] tt-3′ |
| | 100% PTO | |
| GpC1745 | 20mer | 5′-tccatga [symbol] ttcctga [symbol] t-3′ |
| | 100% PTO | |

PTO (phosphorothioate) residues are italicised; CpG/GpC motifs shown in bold;

### 2.2 ELISPOT Assays for T Cell Responses

### Preparation of Mouse Splenocytes

Spleens were obtained from immunised mice at 7 days post immunisation or the time point indicated on the figures. Spleens were processed by grinding between glass slides to produce a cell suspension. Red blood cells were lysed by ammonium chloride treatment and debris was removed to leave a fine suspension of splenocytes. Cells were resuspended at a concentration of 4×10⁶/ml in RPMI complete media for use in ELISPOT assays.

### Peptides Used for Murine Studies

For OVA assays peptide SIINFEKL, a dominant CD8 peptide of OVA, was used in assays at a final concentration of 50 nM to measure CD8 responses and peptide TEWTSSNVMEERKIKV was used at a final concentration of 10 μM to measure CD4 responses. For ICS assays Ovalbumin protein was also used to measure CD4 responses at 1 mg/ml. For ELISPOT to detect responses to p7313RNG peptide the CD8 peptide AMQMLKETI was used for stimulation. For detecting responses to Muc1, CD4 peptides GGSSLSYTNPAVMTSANL and GEKETSATQRSSVPS were used at 10 uM, and CD8 peptide SAPDNRPAL was used at 10 nM. The 9-mer peptides used to follow CD8 responses to Gag and RT in mice were AMQLKETI (Gag CD8) and YYPDSKDLI (RT CD8) respectively, and CD4 responses to Gag and RT were followed using IYKRWIILGLNKIVR (Gag CD4) and QWPLTEEKIKALVEI (RT CD4) respectively. Peptide EREVLEWRFDSRLAF (Nef 218) was also tested. These peptides were tested at a final concentration of 10 μM. The peptides were obtained from Genemed Synthesis, South San Francisco.

### Mouse IFNg and IL-2 ELISPOT Assay

Plates were coated with 15 pg/ml (in PBS) rat anti mouse IFNγ or rat anti mouse IL-2 (Pharmingen). Plates were coated overnight at +4° C. Before use the plates were washed three times with PBS. Splenocytes were added to the plates at 4×10⁵ cells/well. Total volume in each well was 200 μl. Plates containing peptide stimulated cells were incubated for 16 hours in a humidified 37° C. incubator.

### Development of ELISPOT Assay Plates.

Cells were removed from the plates by washing once with water (with 1 minute soak to ensure lysis of cells) and three times with PBS. Biotin conjugated rat anti mouse IFNγ or IL-2 (Pharmingen) was added at 1 μg/ml in PBS. Plates were incubated with shaking for 2 hours at room temperature. Plates were then washed three times with PBS before addition of Streptavidin alkaline phosphatase (Caltag) at 1/1000 dilution. Following three washes in PBS spots were revealed by incubation with BCICP substrate (Biorad) for 15-45 mins. Substrate was washed off using water and plates were allowed to dry. Spots were enumerated using an image analysis system devised by Brian Hayes, Asthma Cell Biology unit, GSK or the AID Elispot reader (Cadama Biomedical, UK).

2.3 Flow Cytometry to Detect IFNγ and IL-2 Production from Murine T Cells in Response to Peptide or Protein Stimulation

4×10⁶ splenocytes were aliquoted per test tube, and spun to pellet. The supernatant was removed and samples vortexed to break up the pellet. 0.5 pg of anti-CD28+0.5 μg of anti-CD49d (Pharmingen) were added to each tube, and left to incubate at room temperature for 10 minutes. 1 ml of medium was added to appropriate tubes, which contained either medium alone, or medium with peptide or protein at the appropriate concentration. Samples were then incubated for an hour at 37° C. in a heated water bath. 10 μg/ml Brefeldin A was added to each tube and the incubation at 37° C. continued for a further 5 hours. The programmed water bath then returned to 6° C., and was maintained at that temperature overnight.

Samples were then stained with anti-mouse CD4-PerCP (Pharmingen) and anti-mouse CD8 APC. In the p7313 RNG examples CD4 CyChrome and CD8 biotin were used and samples were washed, and stained with streptavidin-ECD. Samples were washed and 100 μl of Fixative was added from the “Intraprep Permeabilization Reagent” kit (Immunotech) for 15 minutes at room temperature. After washing, 100 μl of permeabilisation reagent from the Intraprep kit was added to each sample with anti-IFNγ-PE+anti-IL-2-FITC (Immunotech). Samples were incubated at room temperature for 15 minutes, and washed. Samples were resuspended in 0.5 ml buffer, and analysed on the Flow Cytometer.

A total of 500,000 cells were collected per sample and subsequently CD4 and CD8 cells were gated to determine the populations of cells secreting IFNγ and/or IL-2 in response to stimulus.

### 2.4 Tetramer Staining and Analysis

100 μl of whole blood or splenocytes in suspension, were added to each tube. 5 μl of H2-Kb SIINFEKL tetramer (Immunomics) labelled with Phycoeritherin (PE) was added for 20 minutes at room temperature. Anti-mouse CD8-CyChrome or APC was added and left to incubate for a further 10 minutes. If whole blood was analysed, the red blood cells were lysed with “Whole blood lysing solution” (Immunotech) following the manufacturers instructions. After washing the samples were resuspended in buffer and analysed on the Flow Cytometer. 400,000 events were collected per sample.

### 3 Minipig Data

### Immunisation of Minipigs

Minipigs were immunised by delivery of 4 cartridges into the ventral abdomen. Fourteen days later peripheral blood samples were collected for preparation of peripheral blood mononuclear cells (BMC).

### Purification of Porcine PBMC

Porcine blood was collected into heparin, diluted 2:1 in PBS and layered over Histopaque (Sigma) in 50 ml Falcon tubes. The tubes were centrifuged at 1200g for 30 minutes and the porcine lymphocytes harvested from the interface. Residual red blood cells were lysed using ammonium chloride lysis buffer. Cells were counted and resuspended in complete RPMI medium at 2×10⁶/ml.

### Porcine IFNg ELISPOT Assay

Plates were coated with 8 pg/ml (in PBS) (purified mouse anti-swine IFN-□, Biosource ASC4934). Plates were coated overnight at +4° C. Before use the plates were washed three times with PBS and blocked for 2 hours with complete RPMI medium. PBMC were added to the plates at 2×10⁵ cells/well. Total volume in each well was 200 μl. Recombinant Gag, Nef or RT protein (prepared in house) was added at a final concentration of 5 ug/ml. Plates were incubated for 16 hours in a humidified 37° C. incubator.

### Development of ELISPOT Assay Plates.

Cells were removed from the plates by washing once with water (with 1 minute soak to ensure lysis of cells) and three times with PBS. Biotin conjugated anti-porcine IFNγ was added at 0.5 pg/ml in PBS. Plates were incubated with shaking for 2 hours at room temperature. Plates were then washed three times with PBS before addition of Streptavidin alkaline phosphatase (Caltag) at 1/1000 dilution. Following three washes in PBS spots were revealed by incubation with BCICP substrate (Biorad) for 15-45 mins. Substrate was washed off using water and plates were allowed to dry. Spots were enumerated using the AID Elispot reader (Cadama Biomedical, UK).

### 3 Results

### Imiquimod Increases Immune Response

Mice were immunised with by PMID with 2×0.5 ug p731-RNG (GW825780X) or the control empty vector. Where relevant, 20 μl of 5% Aldara™ Cream (3M) was rubbed into each area of immunisation. The Aldara™ cream was applied 24 hours after immunisation.

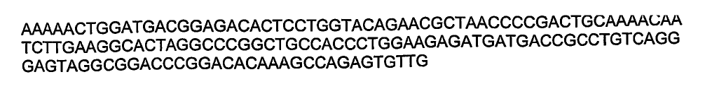

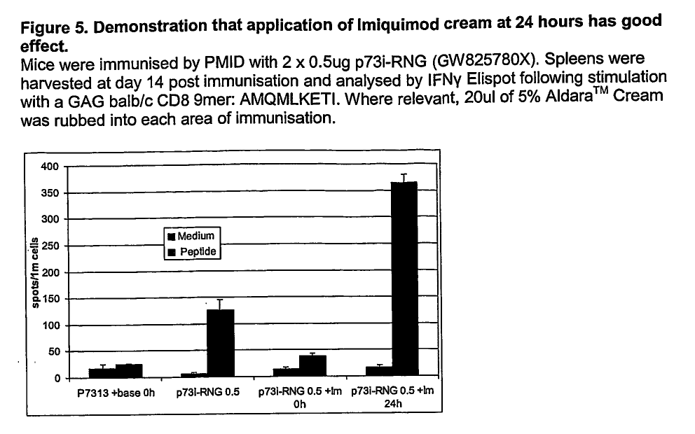

Spleens were harvested at day 14 post immunisation and the cellular responses analysed by IFNγ Elispot following stimulation with a GAG balb/c CD8 9mer peptide: AMQMLKETI. The results are shown in FIG. 5. The data compares delivery of Imiquimod at 0 h or 24 h post immunisation and shows that application 24 h post immunisation has a good adjuvant effect.

### In Vitro Data to Demonstrate Upregulation of TLRs in Response to Inflammatory Stimuli.

### Taqman Analysis of TLR Expression on IFNγ Treated DC.

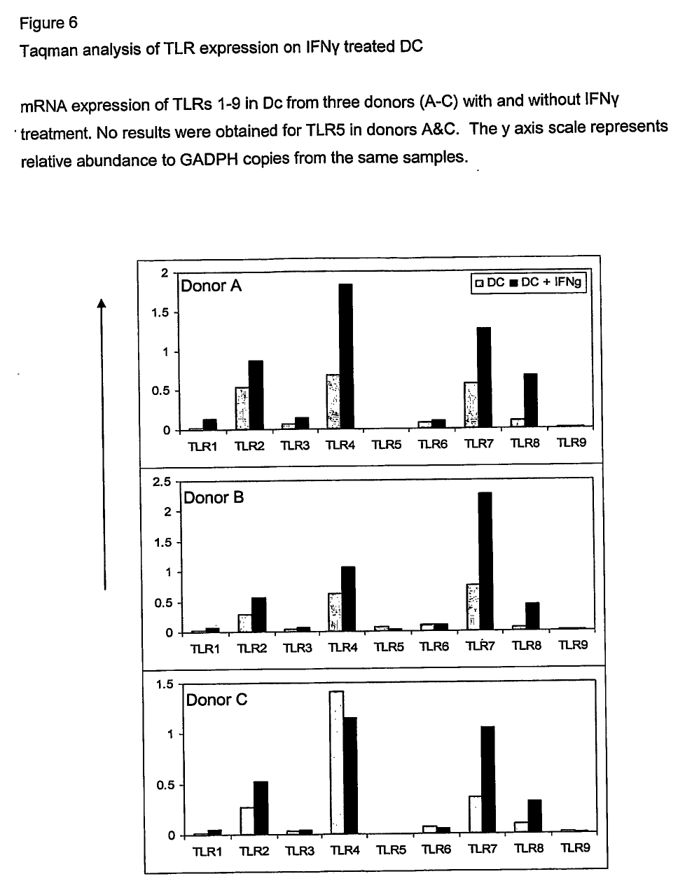

Monocytes were isolated from the PBMC of 3 healthy donors and cultured with IL-4 & GM-CSF for 7 days to induce differentiation to immature DC. The DC were then treated with IFNγ for 24 hours. mRNA expression of TLRs 1-9 was then measured by Taqman. The results are shown in FIG. 6. In contrast to published reports we have shown that low levels of TLR7 are constitutively expressed on monocyte derived DC. Following IFNγ treatment, expression of TLR8 and increased levels of expression of TLR7 were found in all 3 donors. TLR2 was also upregulated but to a lesser extent. The increase in TLR7 expression at 24 hours post stimulation in vitro provides an explanation for the results in FIG. 5 showing the good effect of Imiquimod at 24 hours post immunisation.

### IFNγ Increases the Responsiveness of Dc to Resiquimod.

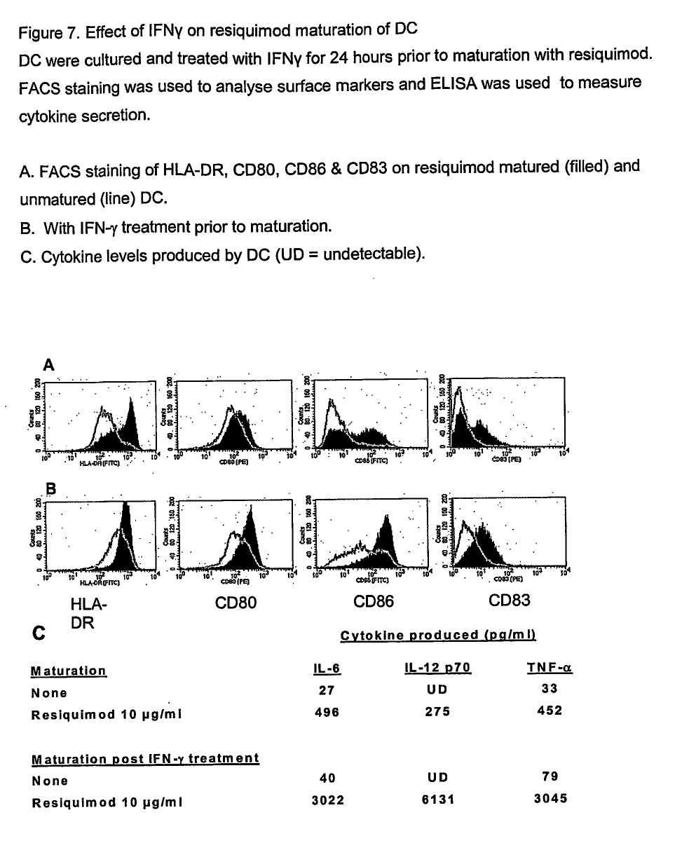

We also investigated the response of cells from these donors to resiquimod. DC were isolated and cultured with GMCSF as before. The DC were then treated with IFNγ for 24 hours, or left untreated, before treatment with resiquimod. Levels of cytokine produced and surface marker expression were measured. The results are showed in FIG. 7. It was found that IFNγ pre-treatment increased the responsiveness of these DC to resiquimod. The maturation process was augmented, resulting in increases in expression of cell surface markers, cytokine production and functional capacity of the DC. These results indicate that TLR7 and TLR8 are involved in the response to resiquimod in human monocyte derived DC, again supporting the delivery of imiquimod at 24 hours post immunisation.

GMCSF Co-Delivery and Imiquimod Application Enhances Cellular Responses to p7313OVAcyt Following Primary Immunisation.

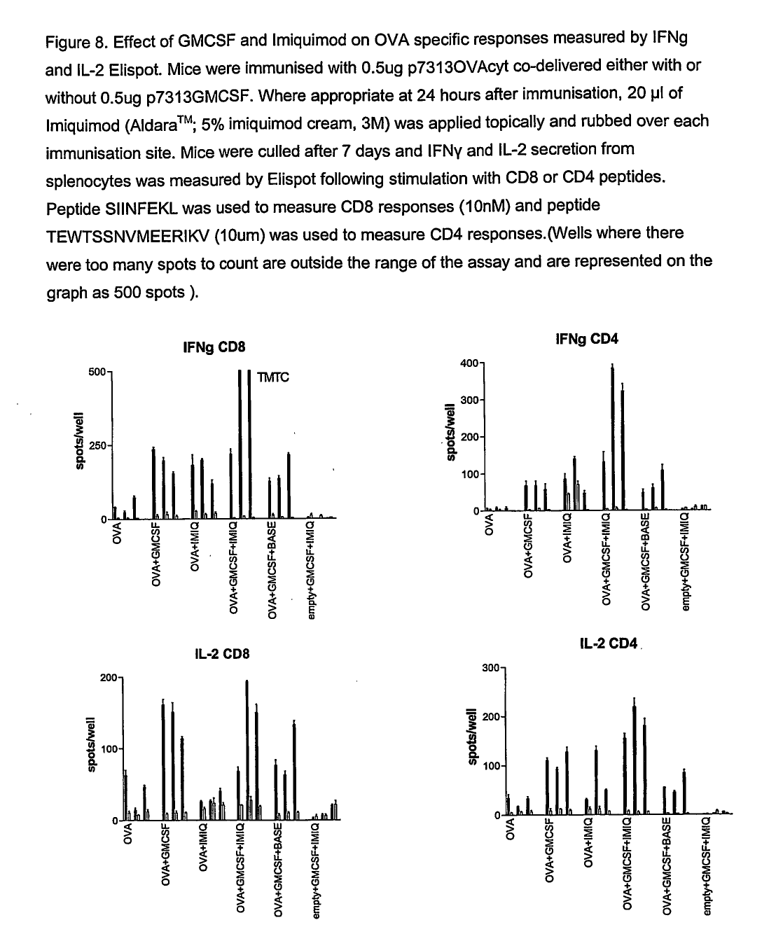

The cellular responses following immunisation with OVAcyt and combinations with p7313GMCSF and imiquimod were assessed by ELISPOT following a primary immunisation by PMID at day 0. Cartridges were loaded with 0.5 μg p7313OVAcyt and 0.5 μg p7313GMCSF or empty vector control. The total DNA dose per mouse given as 2 shots was therefore 2 μg. Assay conditions were: stimulation with SIINFEKL, a high affinity CD8 peptide, or TEWTSSNVMEERKIKV, which contains a CD4 epitope. The results of the Elispot assays are shown in FIG. 8, which shows adjuvant effects when either GMCSF or imiquimod are delivered with p7313 OVAcyt. The analysis was carried out at Day 7 post immunisation. In the OVA+GMCSF+Imiquimod group, the wells in the CD8 IFNγ Elispot contained more spots than could be distinguished for counting, representing a large increase from either GMCSF or imiquimod alone. The other parameter which was improved dramatically compared to immunisation with p7313OVAcyt alone was number of CD4 cells and the proportion of the CD4 cells secreting IFNγ.

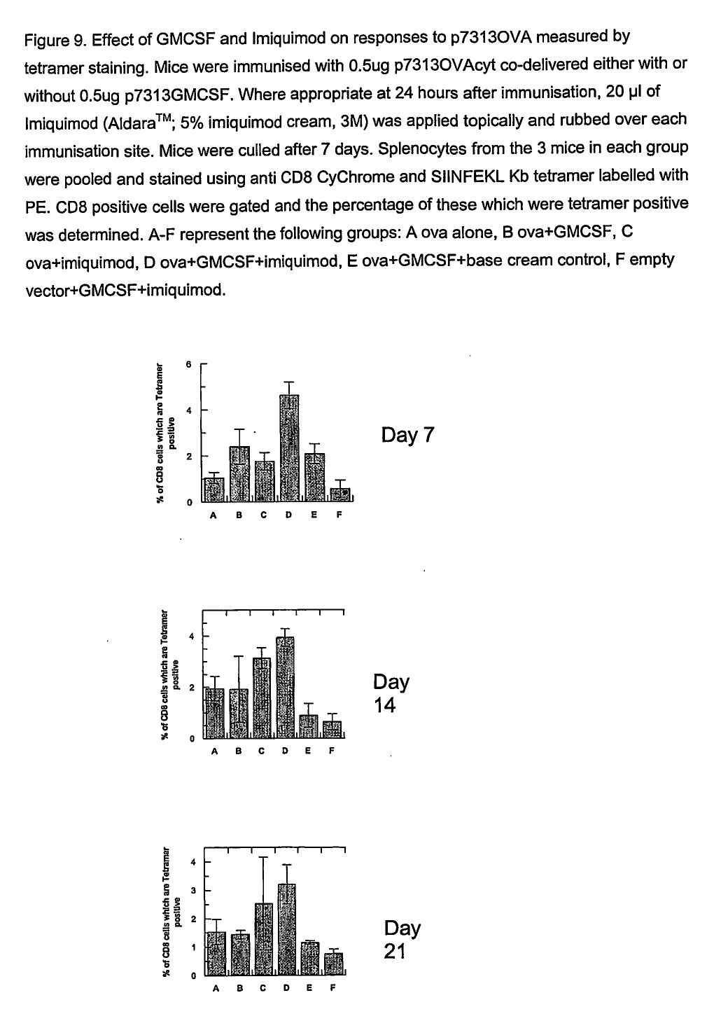

In further experiments following the same immunisation schedule, the cellular responses following immunisation with OVAcyt and combinations with p7313GMCSF and imiquimod were assessed by flow cytometry, as this has the capacity to measure a greater range of responses. Assays were carried out on splenocytes at 7, 14 and 21 days post immunisation. Assay conditions were stimulation with SIINFEKL peptide, a high affinity CD8 peptide or Ovalbumin protein which stimulates both CD4 and CD8 cells. The assays carried out were intracellular cytokine staining for frequency of CD4 and CD8 cells secreting IFNγ and IL-2, and SIINFEKL Kb tetramer staining to determine total frequency of responding CD8 cells. FIG. 9 shows the responses measured by tetramer staining at Days 7, 14 and 21 post primary immunisation. In agreement with the previous experiment, it was found that the combination of GMCSF and imiquimod induced a greater frequency of SIINFEKL specific CD8 cells than either of these alone. FIG. 10 shows the proportion of CD4 and CD8 cells secreting IFNγ and/or IL-2. In agreement with the Elispot results, the combination of GMCSF and imiquimod induced the most potent responses. This was the case for cytokine secretion from both CD8 cells and CD4 cells. In particular, the number of CD4 cells secreting both IFNγ and IL-2 was greatly enhanced.

Imiquimod Application in the Presence or Absence of GMCSF Co-Delivery Enhances Cellular Responses to p7313OVAcyt Following Prime and Boost Immunisation.

Mice were immunised at days 0 and 28 with p7313OVAcyt. This was delivered alone or co-delivered with p7313GMCSF, with some groups given Imiquimod application at 24 hours post immunisation. For immunisation schedules with a prime and boost the dose of p7313OVAcyt was reduced to 0.005 pg/cartridge. p7313GMCSF where present was delivered at 0.5 pg/cartridge. Spleens were harvested at day 7 post boost and analysed by Elispot following overnight stimulation with Ovalbumin CD4 and CD8 peptides. It was found that co-delivery of GMCSF combined with administration of Imiqimod at 24 hours enhanced cellular responses and in particular IFNγ production by both CD4 and CD8 cells compared to Ova alone.

### Effect of GMCSF and Imiquimod on Cellular Responses to Muc1

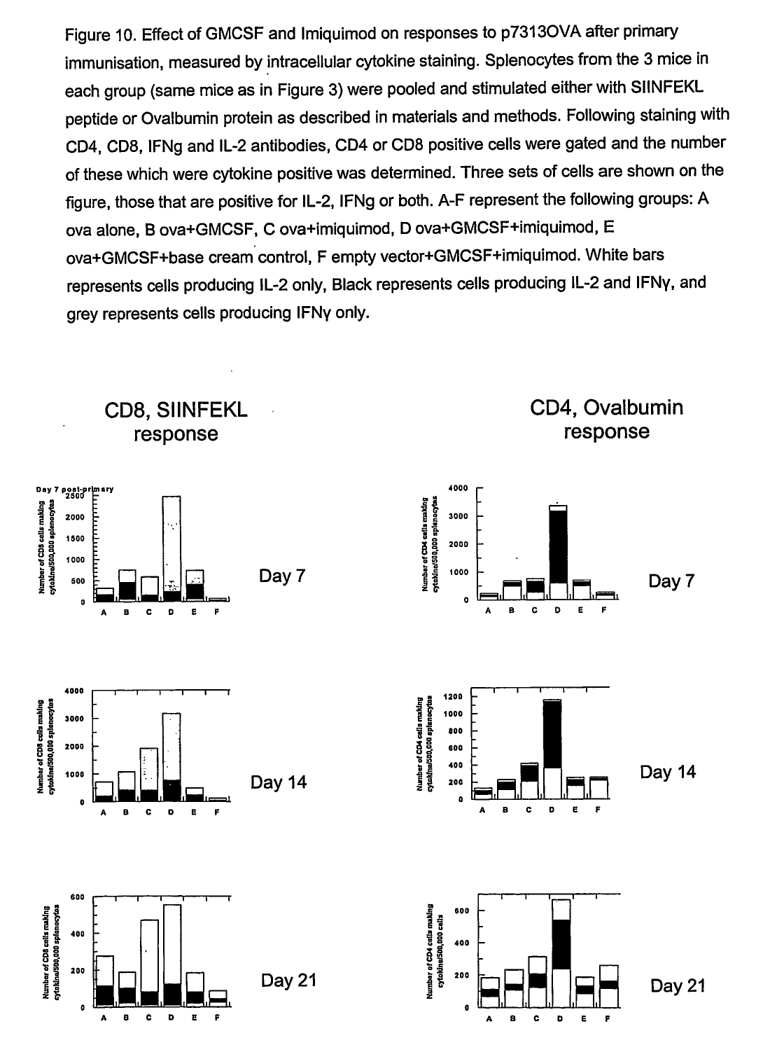

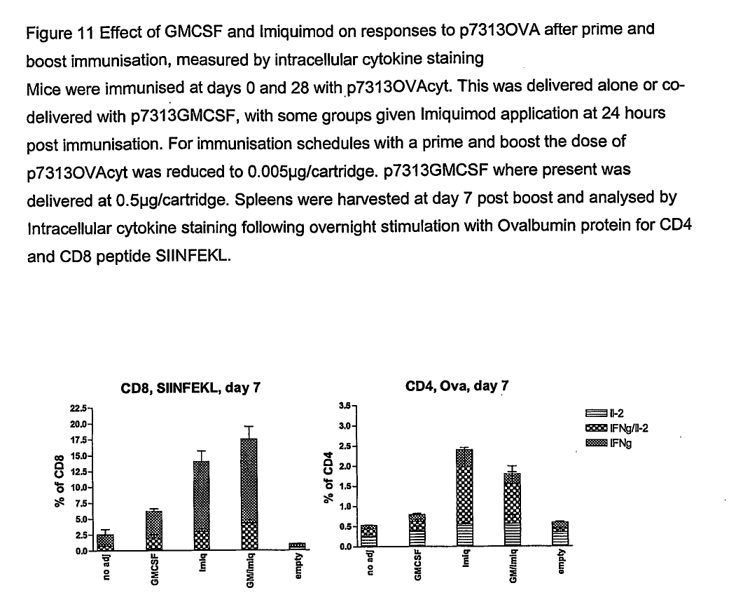

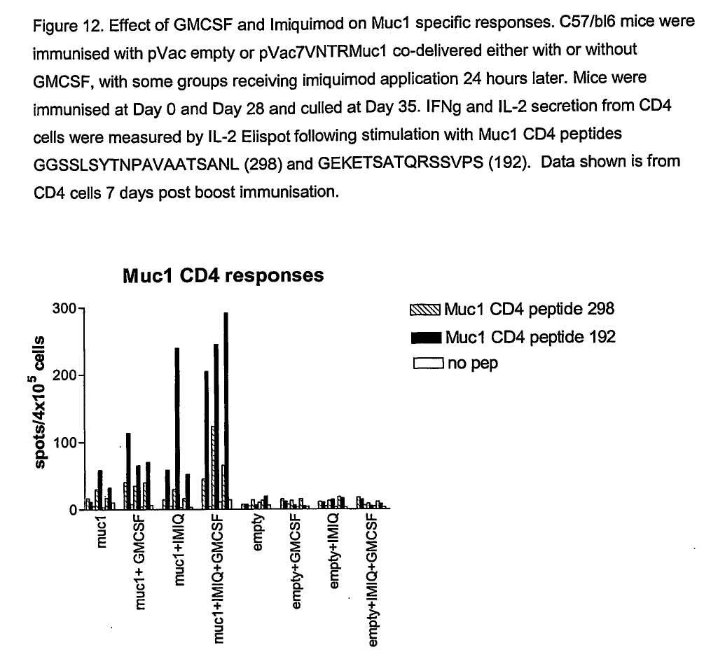

Experiments were carried out to determine the effect of GMCSF and Imiquimod treatment on responses to pVAC7VNTR Muc1. Mice were immunised at days 0 and 21 with pVAC7VNTR muc1. This was delivered alone, co-delivered with p7313GMCSF, or with p7313GMCSF and Imiquimod application at 24 hours post immunisation. Spleens were harvested at day 7 post boost and analysed by Elispot following overnight stimulation with Muc1 CD4 peptides. It was found that co-administration of either p7313 GMCSF or application of imiquimod improved CD4 responses compared to immunisation with pVAC 7VNTRMuc1 alone. Co-delivery of GMCSF combined with administration of imiquimod at 24 hours enhanced responses further (FIG. 12).

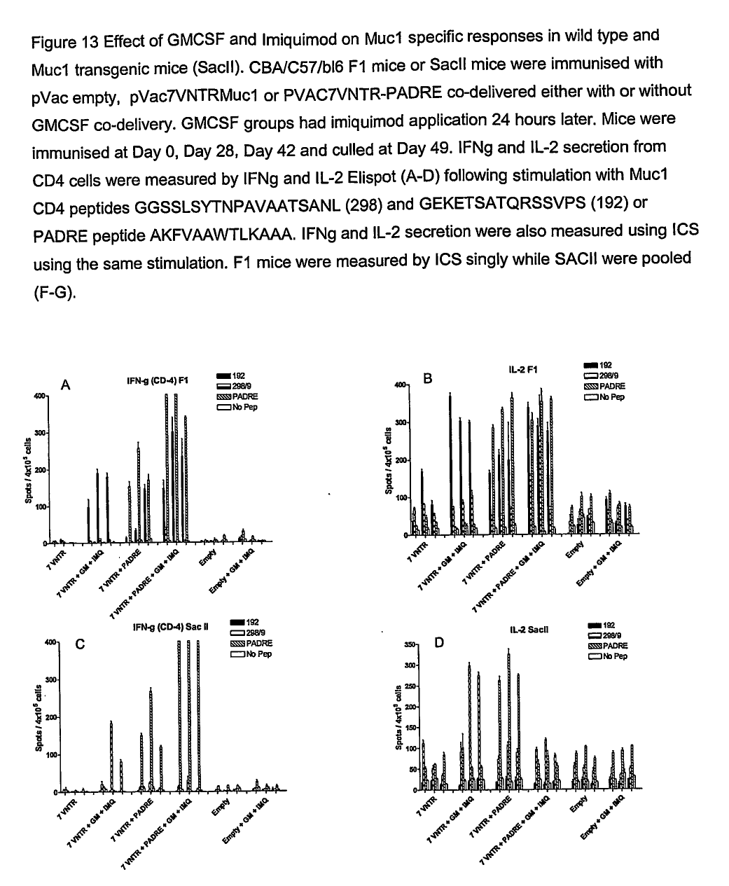

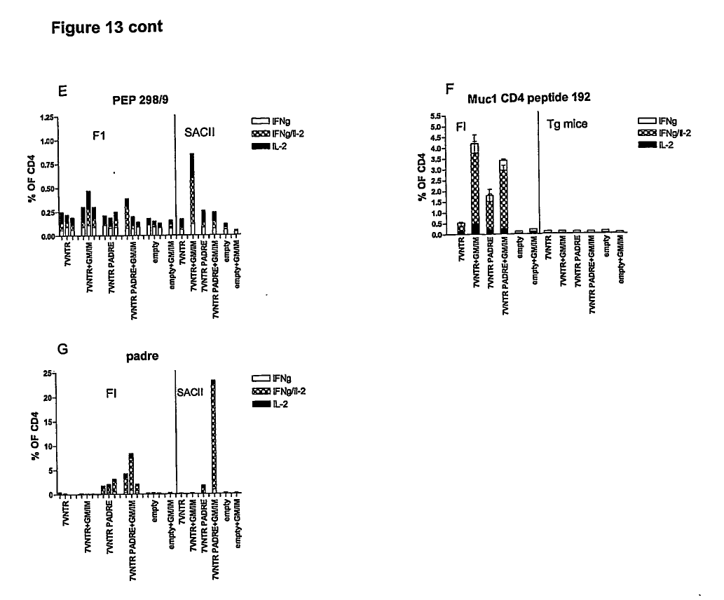

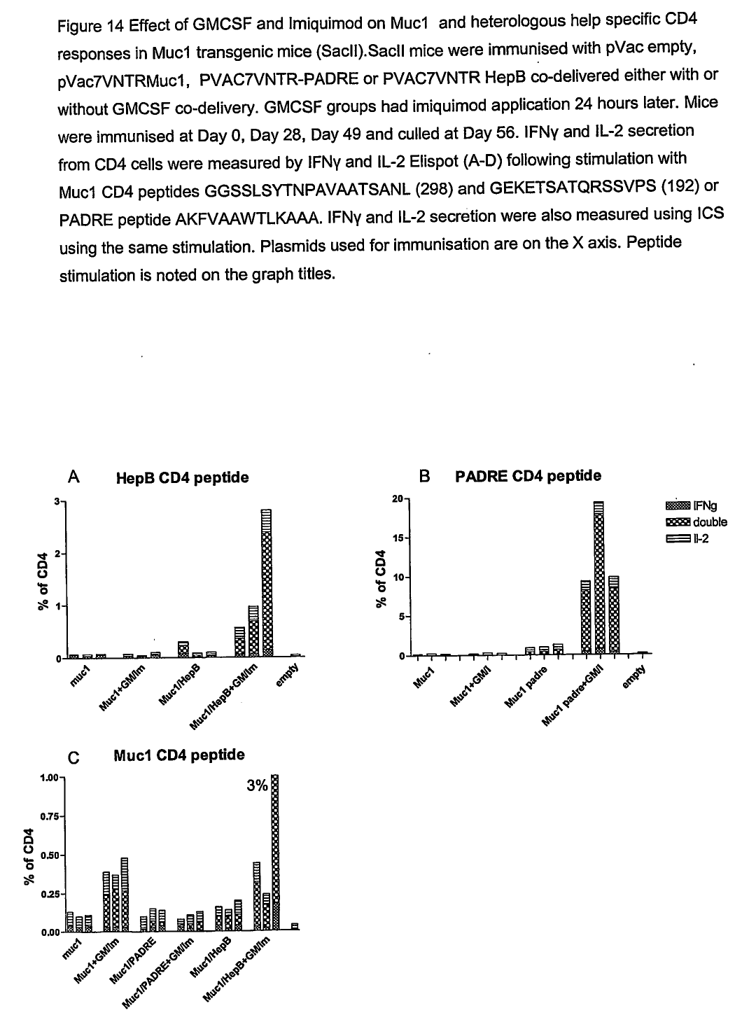

Further experiments were carried out to investigate the effect of GMCSF and Imiquimod on Muc1 responses. For tolerance breaking experiments, Muc1 SacII mice which are transgenic for Human Muc1 were used. These mice are generated on a CBA/C57/bl6 background, so mice with this background were used as controls. CBA/C57/bl6 F1 mice or SacII mice were immunised with pVac empty, pVac7VNTRMuc1 or PVAC7VNTR-PADRE co-delivered either with or without GMCSF co-delivery. GMCSF groups had imiquimod application 24 hours later. Mice were immunised at Day 0, Day 28, Day 42 and culled at Day 49. IFNg and IL-2 secretion from CD4 cells were measured by IFNg and IL-2 Elispot following stimulation with Muc1 CD4 peptides GGSSLSYTNPAVAATSANL (298) and GEKETSATQRSSVPS (192) or PADRE peptide AKFVMWTLKAAA. IFNg and IL-2 secretion were also measured using ICS using the same stimulation. Responses in the groups of wild type mice which received p7313 GMCSF and imiquimod had the highest CD4 responses. This was true for responses to the PADRE peptide or Muc1 peptide. SacII mice immunised with 7VNTRMuc1+GMCSF/Imiquimod had Muc1 CD4 responses to peptide GGSSLSYTNPAVMTSANL (298) so tolerance was broken in these mice. SacII mice immunised with pVac7VNTR PADRE+GMCSF imiquimod had high responses to PADRE (24% of CD4 cells) but no tolerance breaking to Muc1. This may be due to immunodominance of the PADRE response over the Muc1 response (FIG. 13). In a further experiment using an identical protocol (FIG. 14) SacII mice were immunised with pVac empty, pVac7VNTRMuc1, PVAC7VNTR-PADRE or PVAC7VNTR HepB co-delivered either with or without GMCSF co-delivery. In this experiment CD4 responses to HepB and PADRE were enhanced in the presence of GMCSF and Imiquimod and CD4 tolerance to Muc1 CD4 peptide 298 was broken by the 7VNTR construct and the 7VNTRHepB construct.

GMCSF and Imiquimod enhance responses to HIV antigens encoded by p7313RNG plasmid. Female Balb/c (K2^{d}) mice were immunised by delivering 2 cartridges by PMID using a Powderject research device. Two doses of antigen were used, 0.5 and 0.05 ug per cartridge. Where appropriate at 24 hours after immunisation, Imiquimod was applied. Three mice per group were culled at 7 days after immunisation and spleens were removed for analysis of cellular responses by the ELIspot assay. The 9-mer peptides used to follow CD8 responses to Gag and RT were AMQLKETI (Gag CD8) and YYPDSKDLI (RT CD8) respectively, and CD4 responses to Gag and RT were followed using IYKRWIILGLNKIVR (Gag CD4) and QWPLTEEKIKALVEI (RT CD4) respectively. Peptide EREVLEWRFDSRLAF (Nef 218) was also tested. Responses to Gag and RT CD4 and CD8 peptides were enhanced to the greatest extent in the presence of GMCSF combined with Imiquimod in comparison to either of these alone. These results are in agreement with the ovalbumin and Muc1 data where the GMCSF/Imiquimod combination has a strong effect on CD4 cells specifically.

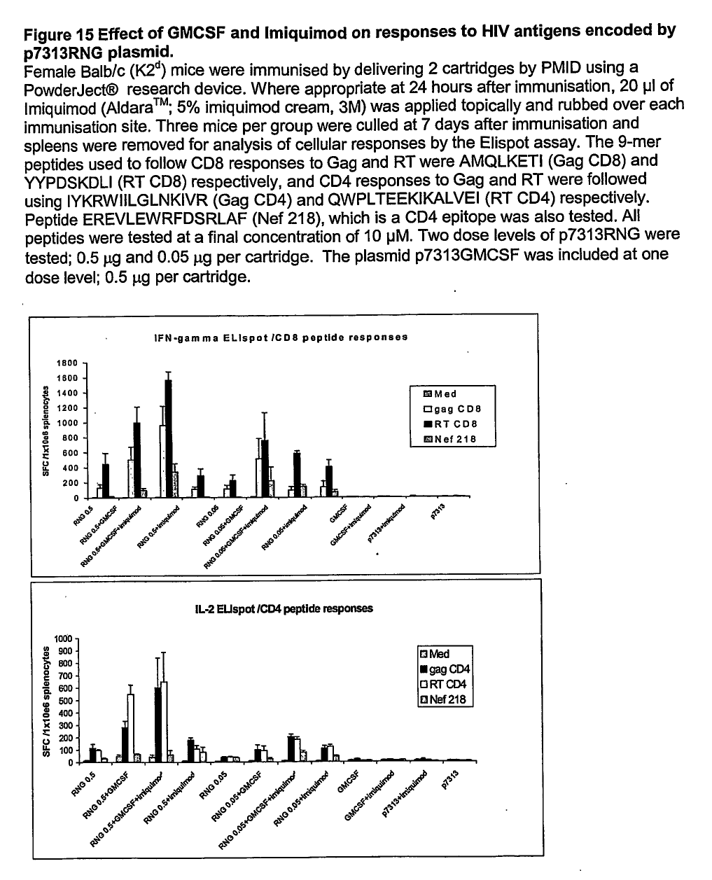

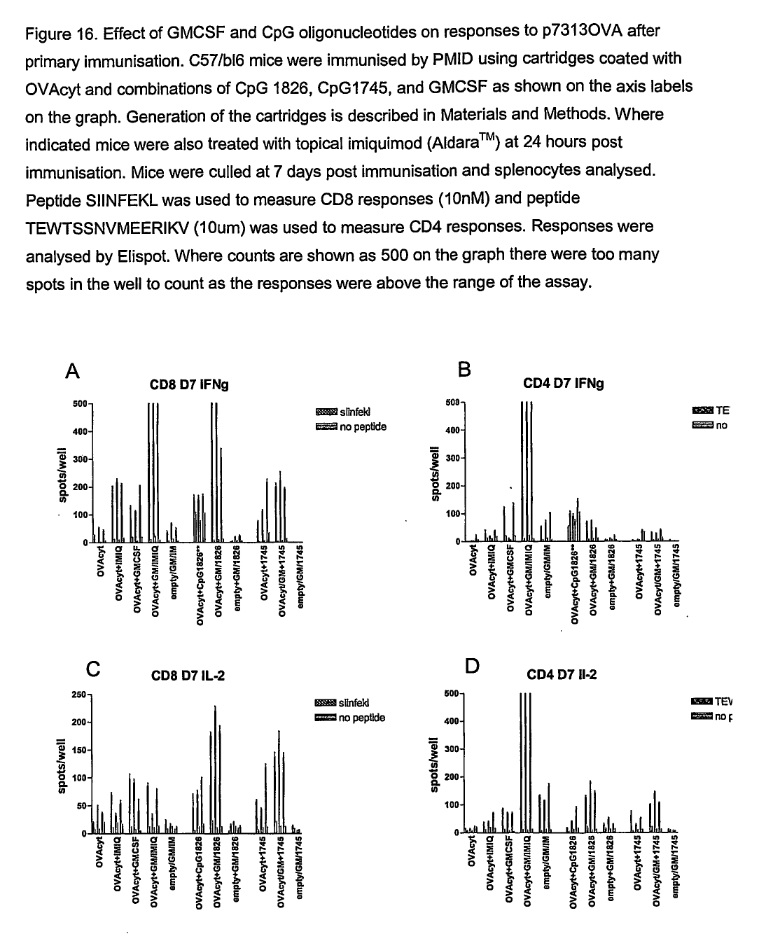

GMCSF and CpG oligonucleotides enhance responses to p7313OVA after primary immunisation. C57/bl6 mice were immunised by PMID using cartridges coated with OVAcyt and combinations of CpG 1826, CpG1745, and GMCSF as shown on the axis labels on the graph. Generation of the cartridges is described in Materials and Methods. Where indicated mice were also treated with topical imiquimod (Aldara™) at 24 hours post immunisation. Mice were culled at 7 days post immunisation and splenocytes analysed. Peptide SIINFEKL was used to measure CD8 responses (10 nM) and peptide TEWTSSNVMEERIKV (10 um) was used to measure CD4 responses (FIG. 16). Co coating of CpG oligo 1826 with p7313OVAcyt was shown to have a positive effect on CD8 responses as measured by the SIINFEKL peptide. CpG 1745, the negative control oligo had a non specific adjuvant effect but this was greatly reduced compared to the 1826. The synergy of the TLR ligand CpG 1826 with GMCSF was similar to that found with Imiquimod.

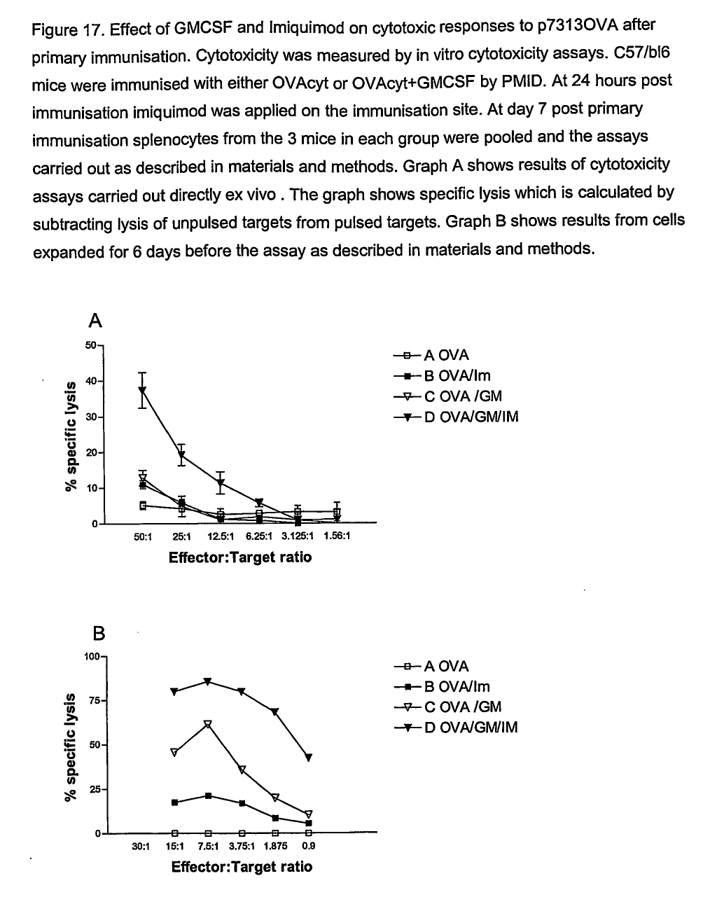

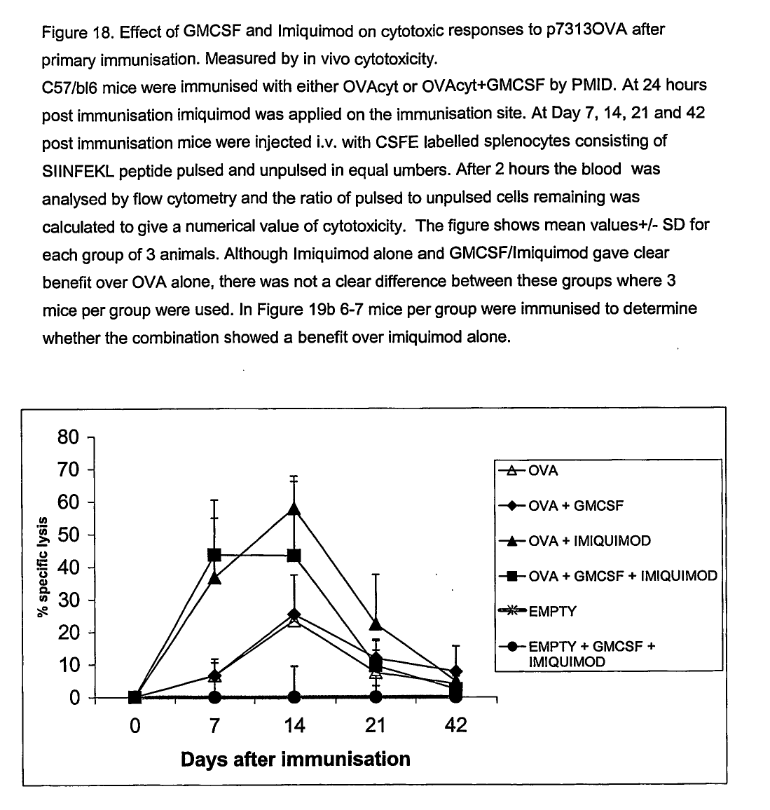

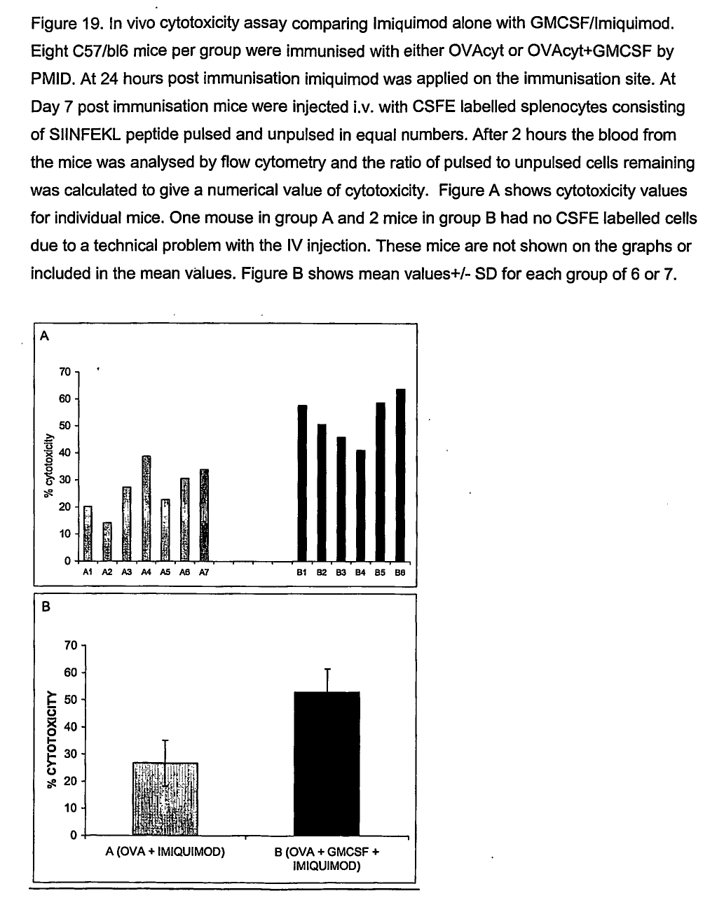

GMCSF and Imiquimod enhance cytotoxic responses to p7313OVA after primary immunisation. C57/bl6 mice were immunised with either OVAcyt or OVAcyt+GMCSF by PMID. At 24 hours post immunisation imiquimod was applied on the immunisation site. At day 7 post primary immunisation splenocytes from the 3 mice in each group were pooled and Cytotoxicity was measured by in vitro cytotoxicity assays as described in materials and methods. Assays were carried out both directly ex vivo and following a 7 day expansion. In both conditions the highest cytotoxicity was found in the GMCSF+Imiquimod group showing that the increase in numbers of responding T cells is functionally relevant (FIG. 17). The effect of GMCSF and Imiquimod on cytotoxic responses to p7313OVA after primary immunisation was also measured by in vivo cytotoxicity assays (FIG. 18). C57/bl6 mice were immunised with either OVAcyt or OVAcyt+GMCSF by PMID. At 24 hours post immunisation imiquimod was applied on the immunisation site. At Day 7, 14, 21 and 42 post immunisation mice were injected i.v. with CSFE labelled splenocytes consisting of SIINFEKL peptide pulsed and unpulsed in equal numbers. After 2 hours the blood was analysed by flow cytometry and the ratio of pulsed to unpulsed cells remaining was calculated to give a numerical value of cytotoxicity. Although Imiquimod alone and GMCSF/Imiquimod gave clear benefit over OVA alone, there was not a clear difference between these groups where 3 mice per group were used. For this reason further experiments were set up in which 6 or 7 mice per group were compared. In this experiment a clear difference in the % of specific lysis was found between the groups, with all the mice in the GMCSF+Imiquimod group showing higher specific lysis than those in the Imiquimod only group (FIG. 19b).

Breaking Tolerance in RIP OVAlo Mice with GM-CSF+Imiquimod

RIP OVAlo mice were used to test the potential for tolerance breaking of the GMCSF+Imiquimod combination (FIG. 20). RIP OVAlo mice express ovalbumin (OVA) on the insulin producing beta cells of the pancreas and are therefore tolerant to this molecule. Disruption of this tolerance results in autoimmune destruction of the beta cells leading to diabetes which can be easily monitored by measurement of glycosuria and blood glucose level. RIPova lo and C57/BL6 mice (wt control group) received four immunisations with empty vector or OVAcyt (using PMID), ±GM-CSF (using PMID), and ±Imiquimod. Immunisations were given at 3 weeks intervals. Imiquimod was applied topically on the site of immunisation 24 h after PMID. 7 Days after the last immunisation splenocytes and serum samples were taken. IFNγ and IL2 production in CD4+ T cells were monitored by intracellular cytokine staining on splenocytes restimulated with TEWTSSNVMEERIKV peptide. IFNγ and IL2 production in CD8+ T cells were monitored by intracellular cytokine staining on splenocytes restimulated with SIINFEKL peptide. H-2 Kb SIINFEKL tetramer analysis of CD8+ T cells was carried out on splenocytes. The results show that to break CD4 tolerance GMCSF+Imiquimod is required (FIG. 20A). In the case of CD8 cells there are responses in the GMCSF alone and Imiquimod alone groups but the responses are highest in the GMCSF+Imiquimod group. This is also the case where CD8 responses are monitored by tetramer (FIG. 20C). The functional test for tolerance breaking in this model is development of diabetes. This is measured by urine glucose levels. Using this test, the immunisation schedule combining GMCSF and Imiquimod is clearly superior FIG. 20E). This experiment shows the importance of inclusion of GMCSF in schedules involving multiple boosts where the aim is tolerance breaking including the generation of functional responses.

GM-CSF and Imiquimod Enhances Primary Responses to p7313RNG (GW825780X) in the Minipig.

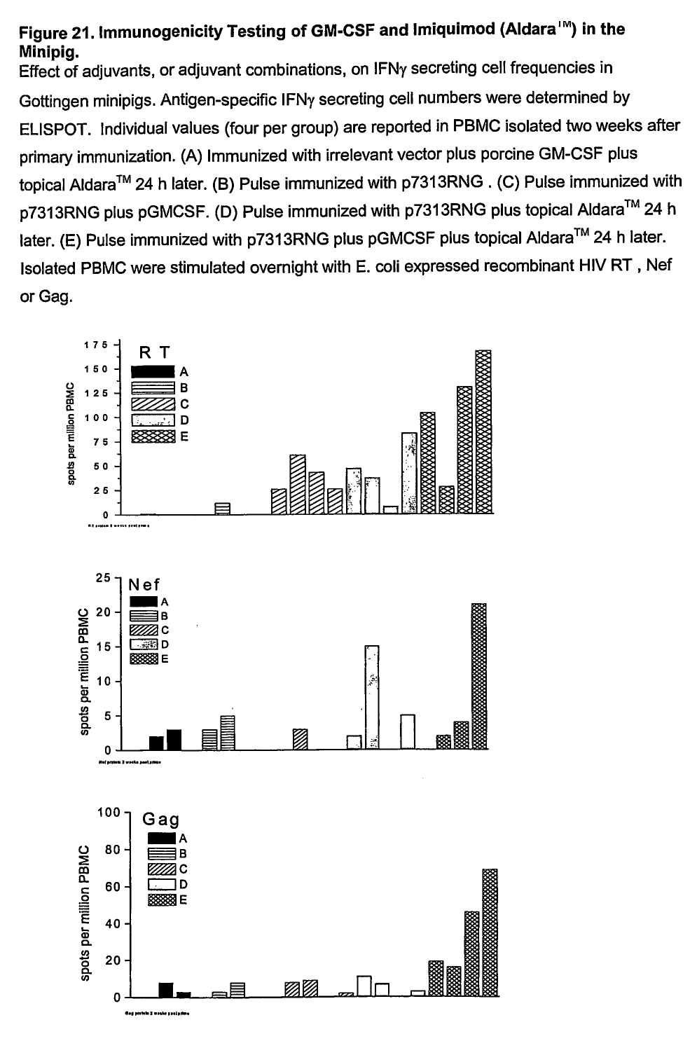

Gottingen minipigs were immunised with 4 administrations (ie. 4 cartridges) on the ventral abdomen. Each cartridge was composed of 0.5 μg p7313RNG and 0.5 μg of either p7313empty or p7313GMCSF (as detailed in the legend to FIG. 21). Fourteen days after the initial immunisation, blood was sampled, PBMC were purified and antigen-specific IFNγ secreting cell numbers were determined by ELISPOT (FIG. 21). The results show that there is an adjuvant effect mediated by the GMCSF+Imiquimod combination which is greater than that mediated by either GMCSF or Imiquimod alone.

The present inventors have determined that the advantage of an adjuvant comprising nucleotide encoding GM-CSF, together with a TLR agonist, is that the adjuvant system of the present invention leads to full activation and maturation of dendritic cells. This in turn leads to a much improved primary immune response against an antigen encoded by a nucleotide sequence. This improvement can be measured by numbers of specific cells and cytotoxic activity. Further, the risk of tolerising the immune system to an antigen, or causing anergy, is much reduced. Additionally, the adjuvant system is capable of overcoming tolerence to self-antigens encoded by nucleotide sequences when administered as a series of immunisations.

1. An adjuvant composition comprising:
(i) a TLR agonist, or nucleotide sequence encoding a TLR agonist; and
(ii) a nucleotide sequence encoding GM-CSF.

2. The adjuvant composition of claim 1 in which the nucleotide sequence encoding component (i) and the nucleotide sequence encoding component (ii), are comprised or consist within one polynucleotide molecule.

3. The adjuvant composition of claim 1 in which the nucleotide sequence encoding component (i) and the nucleotide sequence encoding component (ii) are encoded by nucleotide sequences which are comprised or consist within different nucleotide molecules, for concomitant or sequential administration.

4. The adjuvant composition of claim 1 in which the nucleotide sequence is DNA.

5. The adjuvant composition of claim 1 which the nucleotide sequence or polynucleotide molecule is encoded within plasmid DNA.

6. The adjuvant composition of claim 1 which adjuvant component (i) is a nucleotide sequence encoding one or more of the following molecules, or a component thereof, capable of acting as a TLR agonist: β-defensin; HSP60; HSP70; HSP90; fibronectin; and flagellin protein.

7. The adjuvant composition of claim 1 in which adjuvant component (i) is one or more of the following, or a component thereof, capable of acting as a TLR agonist: an imidazoquinoline molecule, or a derivative thereof; bacterial flagellin protein; lipoprotein (LP), lipopolysaccharide (LPS) or fragments thereof; polyinosinic-polycytidylic acid (polyl:C); peptidoglycan; CpG oligonucleotides; Pam₃Cys lipopeptide; zymosan; HSP60, HSP70, HSP90, fibronectin; and loxoribine for concomitant or sequential administration with component (ii).

8. The adjuvant composition of claim 7 in which the imidazoquinoline or derivative thereof is a compound defined by any one of formulae I-VI, as defined in the present specification.

9. The adjuvant composition of claim 7 in which the imidazoquinoline or derivative thereof is a compound defined by formula VI, as defined in the present specification.

10. The adjuvant composition of claim 7 in the imidazoquinoline or derivative thereof is a compound of formula VI selected from the group consisting of
1-(2-methylpropyl)-1H-imidazo[4,5-c]quinolin-4-amine;
1-(2-hydroxy-2-methylpropyl)-2-methyl-1H-imidazo[4,5-c]quinolin-4-amine;
1-(2,hydroxy-2-methylpropyl)-1H-imidazo[4,5-c]quinolin-4-amine;
1-(2-hydroxy-2-methylpropyl)-2-ethoxymethyl-1-H-imidazo[4,5-c]quinolin-4-amine.

11. The adjuvant composition of claim 7 in which the imidazoquinoline or derivative thereof is imiquimod.

12. The adjuvant composition of claim 7 in the imidazoquinoline or derivative thereof is resiquimod.

13. The adjuvant composition of claim 7 in which component (i) is provided in a separate composition from components (ii) and (ii) for concomitant or sequential administration.

14. An immunogenic composition or compositions comprising adjuvant components (i) and (ii) according to claim 1 and (iii) an immunogen component comprising a nucleotide sequence encoding an antigenic peptide or protein.

15. An immunogenic composition according to claim 14 in which component (i) is encoded by a nucleotide sequence.

16. An immunogenic composition or compositions according to claim 15 in which the nucleotide sequences encoding components (i), (ii) and (iii) are comprised or consist within one polynucleotide molecule.

17. An immunogenic composition or compositions according to claim 15 in which the nucleotide sequences encoding components (i), (ii) and (iii) are comprised or consist within separate polynucleotide molecules, for concomitant or sequential administration.

18. An immunogenic composition or compositions according to claim 15 in which the nucleotide sequences encoding any two of the components (i), (ii) and (iii) are comprised or consist within one polynucleotide molecule, and the remaining nucleotide sequence is encoded within a further polynucleotide molecule, for concomitant or sequential administration.

19. An immunogenic composition or compositions according to claim 18 in which the nucleotide sequences encoding components (ii) and (iii) are comprised or consist within one polynucleotide molecule, and the nucleotide sequence encoding component (i) is encoded within a further polynucleotide molecule, for concomitant or sequential administration.

20. An immunogenic composition or compositions according to claim 14 in which the nucleotide sequence is DNA.

21. An immunogenic composition or compositions according to claim 20 in which the nucleotide sequence or polynucleotide molecule is encoded within plasmid DNA.

22. An immunogenic composition or compositions according to claim 14 in which the nucleotide sequence encodes a P501S protein or derivative which is capable of raising an immune response in vivo, the immune response being capable of recognising a P501S expressing tumour cell or tumour.

23. An immunogenic composition or compositions according to claim 14 in which the nucleotide sequence encodes a MUC-1 protein or derivative which is capable of raising an immune response in vivo, the immune response being capable of recognising a MUC-1 expressing tumour cell or tumour.

24. An immunogenic composition or compositions according to claim 23 in which the MUC-1 protein or derivative is devoid of any repeat units (perfect or imperfect).

25. An immunogenic composition or compositions according to claim 23 in which the MUC-1 protein or derivative is devoid of any perfect repeat units.

26. An immunogenic composition or compositions according to claim 23 in which the MUC-1 protein or derivative contains between one and 15 repeat units.

27. An immunogenic composition or compositions according to claim 23 in which the MUC-1 protein or derivative contains 7 perfect repeat units.

28. An immunogenic composition or compositions according to claim 23 in which the nucleotide sequence encoding the MUC-1 protein or derivative is codon-modified.

29. An immunogenic composition or compositions according to claim 23 in which the nucleotide sequence encoding the non-perfect repeat region has a RSCU of at least 0.6.

30. An immunogenic composition or compositions according to claim 23 in which the nucleotide sequence encoding the non-perfect repeat units of the MUC-1 protein or derivative has a level of identity with respect to wild-type MUC-1 DNA over the corresponding non-repeat regions of less than 85%.

31. An immunogenic composition or compositions according to claim 23 in which the MUC-1 protein or derivative contains altered repeat (VNTR units) such as reduced glycosylation mutants.

32. An immunogenic composition or compositions according to claim 23 in which the MUC-1 protein or derivative is a fusion protein or is conjugated to foreign T-cell epitopes.

33. An immunogenic composition or compositions according to claim 32 in which the MUC-1 protein or derivative is a fusion protein or is conjugated to P2 or P30, or fragments thereof.

34. An immunogenic composition or compositions according to claim 32 in which the foreign T-cell epitopes are incorporated within or at either end of the MUC-1 protein or derivative.

35. A vaccine composition comprising a composition or compositions according to claim 14 and pharmaceutically acceptable carrier(s), diluent(s) or excipient(s).

36. A process for the manufacture of an immunogenic composition comprising mixing adjuvant components (i) and (ii) of claim 1 with an immunogen component (iii) comprising a nucleotide sequence encoding an antigenic peptide or protein.

37. A process according to claim 36 in which adjuvant component (i) is encoded by a nucleotide sequence.

38. A process according to claim 36 in which the nucleotide molecule encoding adjuvant component (ii) is mixed with nucleotide encoding the immunogen component (iii), and adjuvant component (i) is provided in a separate composition for concomitant or sequential administration.

39. A process according to claim 36 in which the nucleotide molecule encoding adjuvant component (ii) is co-encoded with nucleotide encoding the immunogen component (iii) to form a single polynucleotide molecule, and adjuvant component (i) is provided in a separate composition for concomitant or sequential administration.

40. A process according to claim 37 in which the nucleotide sequences encoding components (i), (ii) and (iii) are encoded within separate polynucleotide molecules, for concomitant or sequential administration.

41. A process according to claim 37 in which the nucleotide sequences encoding any two of components (i), (ii) and (iii) are co-encoded to form a single polynucleotide molecule, and the remaining nucleotide sequence is encoded within a further polynucleotide sequence for concomitant or sequential administration.

42. A process according to claim 37 in which the nucleotide sequences encoding components (i), (ii) and (iii) are co-encoded to form a single polynucleotide molecule.

43. A process according to claim 36 in which the nucleotide sequence is DNA.

44. A process according to claim 43 in which the nucleotide sequence is encoded within plasmid DNA.

45. A process according to claim 36 in which the nucleotide molecules encoding components (ii) and (iii) are incorporated within a plasmid, and adjuvant component (i) is provided in a separate composition for concomitant or sequential administration.

46. A process according to claim 36 in which the components are incorporated within pharmaceutically acceptable excipients, diluents or carriers.

47. A pharmaceutical composition or compositions comprising adjuvant components (i) and (ii) according to claim 1 an immunogen component (iii) comprising a nucleotide sequence encoding an antigenic peptide or protein; and one or more pharmaceutically acceptable excipients, diluents or carriers.

48. A pharmaceutical composition or compositions comprising an immunogenic composition or compositions according to claim 14 and pharmaceutically acceptable excipients, diluents or carriers.

49. A kit comprising a pharmaceutical composition comprising adjuvant component (ii); immunogen component (iii), and a pharmaceutical acceptable excipient, diluent or carrier; and a further pharmaceutical composition comprising adjuvant component (i), and a pharmaceutical acceptable excipient, diluent or carrier, in which: adjuvant component (i) comprises a TLR agonist, or a nucleotide encoding a TLR agonist; adjuvant component (ii) comprises a nucleotide encoding GM-CSF; and immunogen component (iii) comprises a nucleotide sequence encoding an antigenic peptide or protein.

50. A pharmaceutical composition or compositions according to claim 47 in which at least one carrier is a gold bead and at least one pharmaceutical composition is amenable to delivery by particle mediated drug delivery.

51. A pharmaceutical composition or compositions according to claim 50 in which the carrier for components (ii) and (iii) is a gold bead and adjuvant component (i) is formulated for concomitant or sequential administration.

52. A method of treating a patient suffering from or susceptible to a tumour, by the administration of a safe and effective amount of an immunogenic, vaccine or pharmaceutical composition according to claim 14.

53. A method of treating a patient according to claim 52, in which the tumour is a MUC-1 expressing tumour.

54. A method of treating a patient according to claim 52, in which the tumour is carcinoma of the breast; carcinoma of the lung, including non-small cell lung carcinoma; or prostate, gastric and other gastrointestinal carcinomas.

55. A method of increasing an immune response of a mammal to an antigen, the method comprising administration of the following components:
(i) a TLR agonist, or a nucleotide encoding a TLR agonist;
(ii) a nucleotide encoding GM-CSF; and
(iii) an immunogen component comprising a nucleotide sequence encoding an antigenic peptide or protein.

56. A method of increasing an immune response according to claim 55, the method comprising concomitant administration of any two of components (i), (ii) and (iii), and sequential administration of the remaining component.

57. A method of increasing an immune response according to claim 55, the method comprising sequential administration of components (i), (ii) and (iii)

58. A method of increasing an immune response of a mammal to an antigen according to claim 55 in which the components for concomitant administration are formulated into separate compositions.

59. An immunogenic composition comprising the following components, in the manufacture of a medicament for use in the treatment or prophylaxis of MUC-1 expressing tumours:
(i) a TLR agonist, or a nucleotide encoding a TLR agonist;
(ii) a nucleotide encoding GM-CSF; and
(iii) an immunogen component comprising a nucleotide sequence encoding an antigenic peptide or protein.

60. A method of raising an immune response in a mammal against a disease state, comprising administering to the mammal within an appropriate vector, a nucleotide sequence encoding an antigenic peptide associated with the disease state; additionally administering to the mammal within an appropriate vector, a nucleotide sequence encoding GM-CSF; and further administering to the mammal an imidazoquinoline or derivative thereof to raise the immune response.

61. A method of increasing the immune response of a mammal to an immunogen, comprising the step of administering to the mammal within an appropriate vector, a nucleotide sequence encoding the immunogen in an amount effective to stimulate an immune response and a nucleotide sequence encoding GM-CSF; and further administering to the mammal an imidazoquinoline or derivative thereof in an amount effective to increase the immune response.

62. (canceled)

63. (canceled)

64. (canceled)

65. (canceled)
